(19) United States
(12) Patent Application Publication
(10) Pub. No.: US 2008/0091065 A1
(43) Pub. Date: Apr. 17, 2008

(54) Medical image processing apparatus, endoscope system and medical image processing system

(75) Inventors: Ryu Oshima, Tokyo (JP); Hiroshi Tamai, Tokyo (JP); Soichiro Imoto, Tokyo (JP); Yuji Horie, Tokyo (JP); Sumihiro Uchimura, Sagamihara-shi (JP); Taiji Uchida, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corporation, Tokyo (JP)

(21) Appl. No.: 11/906,739

(22) Filed: Oct. 3, 2007

(30) Foreign Application Priority Data
Oct. 4, 2006 (JP) 2006-273401
Oct. 4, 2006 (JP) 2006-273402
Oct. 4, 2006 (JP) 2006-273403

(51) Int. Cl. A61B 1/04

(52) U.S. Cl. 600/109; 348/65; 382/128

(57) ABSTRACT

The medical image processing apparatus according to the present invention includes an image compressing unit for compressing a medical image with either a first image compressing method or a second image compressing method; a record direction input setting unit for allocating a first record directing section that is capable of performing a first direction for compressing the medical image with the first image compressing method and a second record directing section that is capable of performing a second direction for compressing the medical image with the second image compressing method; and a controlling unit for outputting the medical image that is compressed with the first image compressing method to an image recording unit in response to detection of the first direction and outputting the medical image that is further compressed with the second image compressing method to the image recording unit in response to detection of the second direction.

### CROSS REFERENCE TO RELATED APPLICATION

This application claims benefit of Japanese Applications No. 2006-273401 filed on Oct. 4, 2006, and, No. 2006-273402 filed on Oct. 4, 2006, and, No. 2006-273403 filed on Oct. 4, 2006, the contents of which are incorporated by this reference.

### BACKGROUND OF THE INVENTION

1. Field of the Invention

The present invention relates to medical image processing apparatus, and more specifically to medical image processing apparatus that is capable of selecting an image compressing method on an obtained medical image.

The present invention also relates to an endoscope system, and more specifically to an endoscope system that is capable of adding functionality.

The present invention further relates to a medical image processing system, and more specifically to a medical image processing system that is capable of recording a first medical image and a second medical image displayed on a display unit.

2. Description of the Related Art

Endoscope systems employing an endoscope, medical image processing apparatus and the like have been used in the medical field and the like. Particularly, endoscope systems employed in the medical field are mainly used in a case where an operator and the like are observing inside a living body. As an appliance used in such endoscope systems, the endoscope file device disclosed in Japanese Patent Laid-Open No. 6-110986, for example, is proposed.

The endoscope file device disclosed in Japanese Patent Laid-Open No. 6-110986 is capable of multi-functionally managing an endoscope image by employing a configuration for compressing image signals outputted from a plurality of endoscopes with at least two compression methods as well as storing respective compressed image signals in a plurality of storage means.

Endoscope systems with an endoscope, a processor and the like have been widely used in the medical fields and the like. Particularly, endoscope systems employed in the medical field is mainly used in a case where an operator and the like are observing inside a living body. As an endoscope system with such a configuration, the electronic endoscope device disclosed in Japanese Patent Laid-Open No. 2004-000335 is proposed.

The electronic endoscope device disclosed in Japanese Patent Laid-Open No. 2004-000335 is capable of improving operability for a user to use the electronic endoscope device by comprising detecting means for detecting a connection status with a signal processing device, and control means for controlling the performance of the signal processing device based on the detected result of the detecting means.

Endoscope systems with an endoscope, medical image processing apparatus and the like have been widely used in the medical fields and the like. Particularly, endoscope systems employed in the medical field are mainly used in a case where an operator and the like are observing inside a living body. As an endoscope system with such a configuration, the endoscope device disclosed in Japanese Patent Laid-Open No. 2002-186582 is proposed.

The endoscope device disclosed in Japanese Patent Laid-Open No. 2002-186582 is capable of displaying an image in an appropriate display state in each case of recording and observing an endoscope image by employing a configuration that enables a display state of character information displayed along with an endoscope image to be changed between a first display state which is a state while character information is not being recorded to recording means and a second display state which is a state while character information is being recorded to the recording means.

Recently, an endoscope system and the like that is capable of displaying two images each with the aspect ratio of 4:3, for example, on a monitor available for wide display of an image with the aspect ratio of 16:9 has also come into use.

### SUMMARY OF THE INVENTION

Medical image processing apparatus in the present invention comprises: an image compressing unit for compressing a medical image according to a subject's image taken by an image pickup unit with either a first image compressing method or a second image compressing method different from the first image compressing method; a record direction input setting unit for allocating a first record directing section that is capable of performing a first record direction for compressing the medical image with the first image compressing method and recording the image and a second record directing section that is capable of performing a second record direction for compressing the medical image with the second image compressing method and recording the image to any of a plurality of keys or switches; and a controlling unit for outputting the medical image that is compressed with the first image compressing method to an image recording unit in response to detection of performance of the first record direction and outputting the medical image that is compressed with the second image compressing method to the image recording unit in response to detection of performance of the second record direction.

An endoscope system in the present invention comprises: an endoscope for picking up an image of a subject; medical image processing apparatus for obtaining an endoscope image according to the subject's image; at least one expansion controlling unit that can be connected to the medical image processing apparatus and that enables at least a predetermined function related with either the endoscope or the medical image processing apparatus when the expansion controlling unit is connected to the medical image processing apparatus; a connection information storing unit that is provided for each of the expansion controlling unit for storing different types of connection detection information according to the type of the expansion controlling unit; and a main controlling unit for determining the type of the expansion controlling unit that is connected to the medical image processing apparatus based on the connection detection information stored in each of the connection information storing unit and outputting an image or information related with the predetermined function according to the determined result to a display unit.

A medical image processing system in the present invention comprises: an endoscope for picking up an image of a subject; medical image processing apparatus that is capable of outputting a first medical image according to the subject's image on an image region in a display unit and outputting a second medical image according to the subject's image on an image region different from the first image region in the display unit; an image recording unit that is capable of recording the first medical image and the second medical image; and a controlling unit for selecting between recording the first medical image and the second medical image as an image on a screen or recording the first medical image and the second medical image as images on different screens according to an outputted size of an image that can be recorded by the image recording unit.

### BRIEF DESCRIPTION OF THE DRAWINGS

FIG. 1A is a diagram showing an example of a configuration of a main part of an endoscope system of the present embodiment;

FIG. 1B is a diagram showing an example of a configuration of an endoscope in the endoscope system shown in FIG. 1A;

FIG. 1C is a diagram showing an example of a configuration of an endoscope in the endoscope system shown in FIG. 1A, the endoscope being different from the endoscope shown in FIG. 1B;

FIG. 1D is a diagram showing an example of a configuration of light equipment in the endoscope system shown in FIG. 1A;

FIG. 1E is a diagram showing an example of a configuration of a processor in the endoscope system shown in FIG. 1A;

FIG. 2A is a diagram showing an example of a configuration of an image processing unit in a processor shown in FIG. 1E;

FIG. 2B is a diagram showing an example of a screen that is displayed when both the endoscope shown in FIG. 1B and the endoscope shown in FIG. 1C are connected to the processor shown in FIG. 1E;

FIG. 2C is a diagram showing an example of a configuration of a main controlling unit in the processor shown in FIG. 1E;

FIG. 2D is a diagram showing an example of a configuration of an expansion controlling unit connected to the processor shown in FIG. 1E;

FIG. 2E is a diagram showing an example of a configuration of an expansion controlling unit different from the expansion controlling unit shown in FIG. 2D connected to the processor shown in FIG. 1E;

FIG. 2F is a diagram showing an example of a configuration of a controller/selector in the processor shown in FIG. 1E;

FIG. 2G is a diagram showing an example of a configuration of a synchronous circuit in an image decompressing unit of the processor shown in FIG. 1E;

FIG. 2H is a flowchart showing an example of processing that the main controlling unit shown in FIG. 2C performs to detect (and when the main controlling unit shown in FIG. 2C detected) connection of the expansion controlling unit;

FIG. 3A is a diagram showing an example of a peripheral device that can be connected to the processor shown in FIG. 1E;

FIG. 3B is a diagram showing an example of a peripheral device that can be connected to the processor shown in FIG. 1E, the example being different from the example shown in FIG. 3A;

FIG. 3C is a diagram showing an example of a peripheral device that can be connected to the processor shown in FIG. 1E, the example being different from the examples shown in FIG. 3A and FIG. 3B;

FIG. 3D is a diagram showing an example of a peripheral device that can be connected to the processor shown in FIG. 1E, the example being different from the examples shown in FIG. 3A, FIG. 3B and FIG. 3C;

FIG. 3E is a diagram showing an example of a peripheral device that can be connected to the processor shown in FIG. 1E, the example being different from the examples shown in FIG. 3A, FIG. 3B, FIG. 3C and FIG. 3D;

FIG. 3F is a diagram showing a simplified inner configuration of some of the peripheral devices shown in FIG. 3A to FIG. 3E;

FIG. 3G is a diagram showing a configuration of data stored in a shared region of each of the memories shown in FIG. 3F;

FIG. 3H is a diagram showing a configuration of data stored in a log region of each of the memories shown in FIG. 3F;

FIG. 3I is a flowchart showing an example of processing related with the data stored in the shared regions shown in FIG. 3G;

FIG. 4 is a diagram showing an example of display size of an image;

FIG. 5 is a diagram showing an example of a configuration of an image compressing unit in the processor shown in FIG. 1E;

FIG. 6 is a diagram showing an example of a configuration of an image decompressing unit in the processor shown in FIG. 1E;

FIG. 7 is a diagram showing an example of an endoscope composite image generated by the image processing unit shown in FIG. 2A;

FIG. 8 is a diagram showing an example of a setting screen of the processor shown in FIG. 1E;

FIG. 9 is a diagram showing an example of another setting screen, which is among the setting screens of the processor shown in FIG. 1E, displayed after transition from the setting screen shown in FIG. 8;

FIG. 10 is a diagram showing an example of a case where a moving image is displayed by PinP;

FIG. 11A is a diagram showing an example of a directory structure used when an image is recorded in each filing device, each optical recording device and the like shown in FIG. 3A to FIG. 3E;

FIG. 11B is a diagram showing an example of a data configuration of an image file of thumbnail images and an image file of images which originated the thumbnail images among files in the directory structures shown in FIG. 11A;

FIG. 11C is a diagram showing another example of a data configuration of an image file of thumbnail images and an image file of images which originated the thumbnail images among files in the directory structures shown in FIG. 11A, the example being different from the example shown in FIG. 11B;

FIG. 12 is a diagram showing an example of a directory name file and a file name displayed on a monitor and the like in a display form corresponding to the directory structure shown in FIG. 11A;

FIG. 13 is a flowchart showing an example of control and processing performed by the main controlling unit shown in FIG. 2C when a still image recorded in the peripheral devices shown in FIG. 3A to FIG. 3E and the like is displayed;

FIG. 14A is a diagram showing an example of multi-images generated by the processing shown in FIG. 13;

FIG. 14B is a diagram showing an example of page switching when multi-images are generated by the processing shown in FIG. 13;

FIG. 14C is a diagram showing an example of screen transition in the multi-images shown in FIG. 14A to display a selected image;

FIG. 15A is a diagram showing an example of processing performed by the processor shown in FIG. 1E when record direction is performed;

FIG. 15B is a diagram showing an example of processing performed by the processor shown in FIG. 1E following the processing shown in FIG. 15A when record direction is performed;

FIG. 15C is a diagram showing an example different from the example shown in FIG. 15B of processing performed by the processor shown in FIG. 1E following the processing shown in FIG. 15A when record direction is performed;

FIG. 15D is a diagram showing an example different from the examples shown in FIG. 15B and FIG. 15C of processing performed by the processor shown in FIG. 1E following the processing shown in FIG. 15A when record direction is performed;

FIG. 15E is a diagram showing an example different from the examples shown in FIG. 15B, FIG. 15C and FIG. 15D of processing performed by the processor shown in FIG. 1E following the processing shown in FIG. 15A when record direction is performed;

FIG. 16 is a diagram showing an example of a screen region that is outputted from a selector of the controller/selector shown in FIG. 2F to a memory and stored in the memory;

FIG. 17 is a diagram showing an example of a thumbnail image generated by the thumbnail image generating section shown in FIG. 2A and a screen region that is taken as a standard for generating the thumbnail image;

FIG. 18A is a flowchart showing an example of compression and recording included in the processing shown in FIG. 15B (FIG. 15C and FIG. 15D);

FIG. 18B is a flowchart showing an example of processing performed when an image in a format of low compression rate, which is stored in a buffer by the processing shown in FIG. 18A, is recorded in a peripheral device and the like;

FIG. 18C is a flowchart showing an example of processing performed when an image in a format of low compression rate, which is stored in a buffer by the processing shown in FIG. 18A, is recorded in a peripheral device and the like, the example being different from the example shown in FIG. 18B;

FIG. 18D is a diagram showing an example of multi-images which are generated for the purpose of selecting an image to be recorded from images stored in the buffer in the processing shown in FIG. 18B;

FIG. 19A is a diagram showing an example of transition of screens displayed on a monitor or the like by the processing shown in FIG. 15B;

FIG. 19B is a diagram showing an example of transition of screens displayed on a monitor or the like by the processing shown in FIG. 15B, the example being different from the example shown in FIG. 19A;

FIG. 19C is a diagram showing an example of transition of screens displayed on a monitor or the like by the processing shown in FIG. 15C;

FIG. 19D is a diagram showing an example of transition of screens displayed on a monitor or the like by the processing shown in FIG. 15C, the example being different from the example shown in FIG. 19C;

FIG. 19E is a diagram showing an example of transition of screens displayed on a monitor or the like by the processing shown in FIG. 15D;

FIG. 19F is a diagram showing an example of transition of screens displayed on a monitor or the like by the processing shown in FIG. 15D, the example being different from the example shown in FIG. 19E;

FIG. 19G is a diagram showing an example of transition of screens displayed on a monitor or the like by the processing shown in FIG. 15D, the example being different from the examples shown in FIG. 19E and FIG. 19F;

FIG. 19H is a diagram showing an example of transition of screens displayed on a monitor or the like by the processing shown in FIG. 15E;

FIG. 20A is a diagram showing an example of processing performed by each unit of the processor shown in FIG. 1E when freeze direction or S freeze direction is done; and

FIG. 20B is a diagram showing an example of processing performed by the processor shown in FIG. 1E following the processing shown in FIG. 20A when S freeze direction is done.

### DETAILED DESCRIPTION OF THE PREFERRED EMBODIMENTS

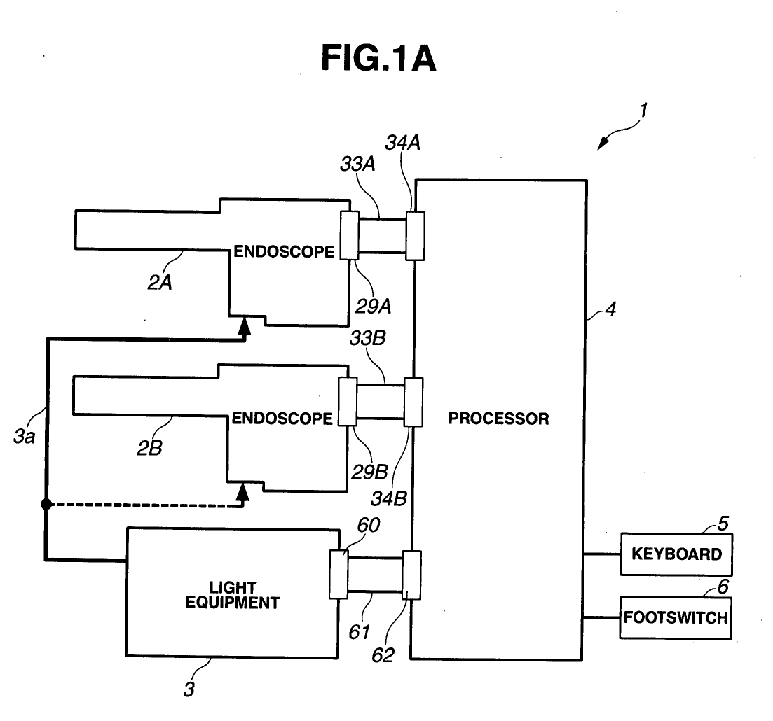

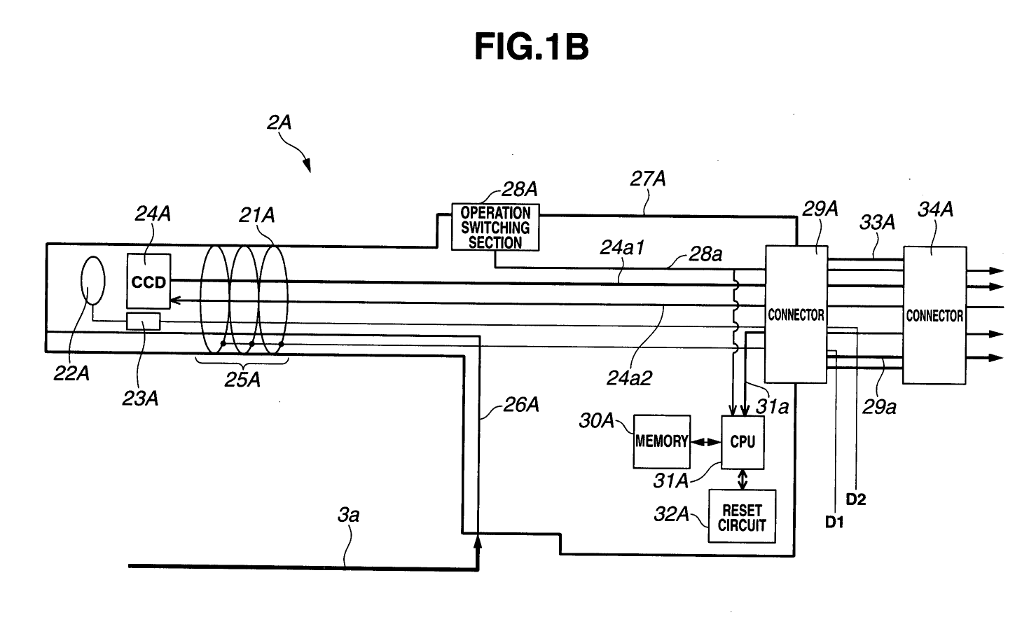

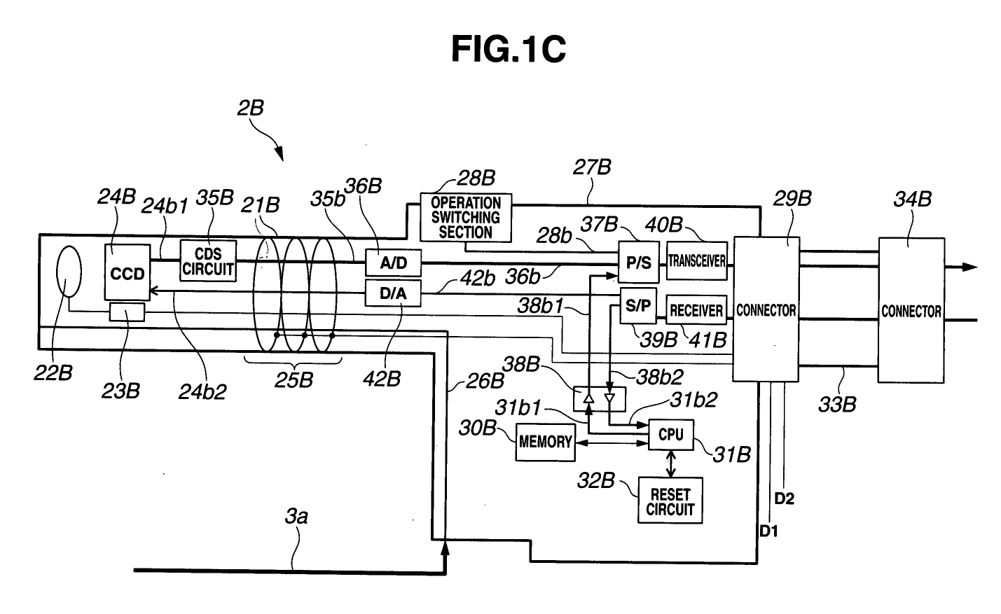

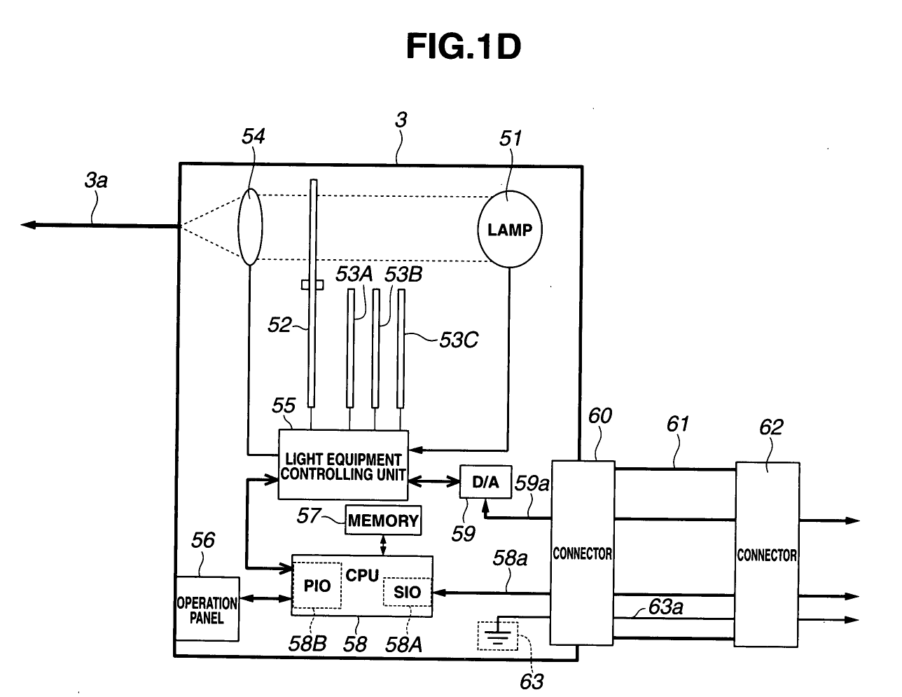

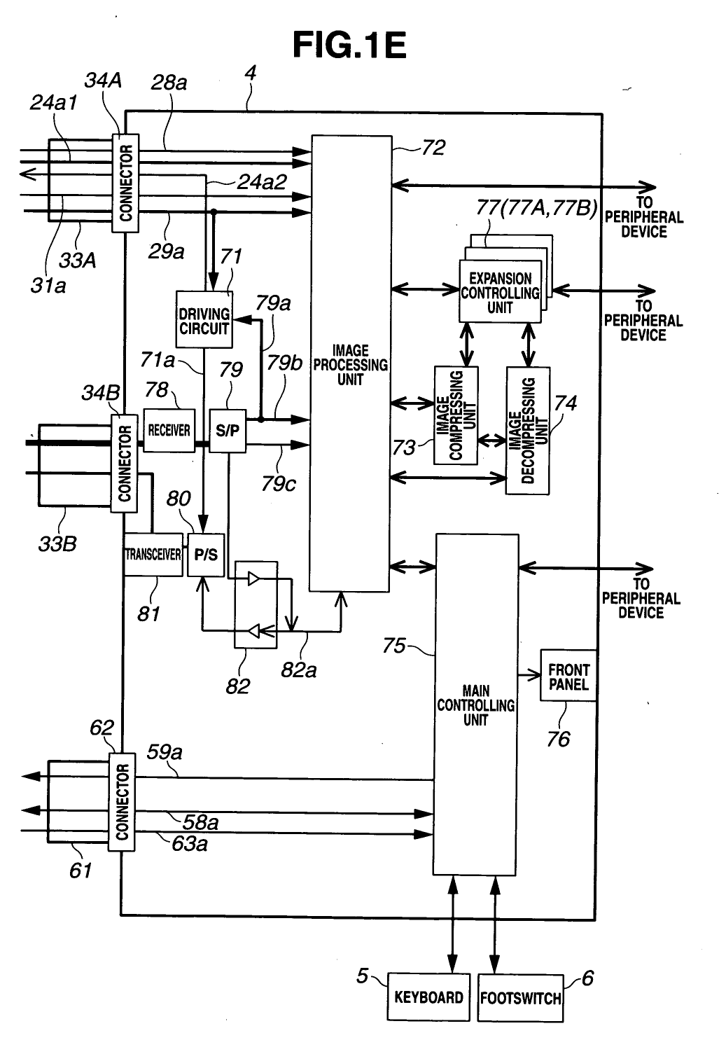

Embodiments of the present invention will be described below with reference to the drawings. FIG. 1A to FIG. 20B are related with the embodiments of the present invention.

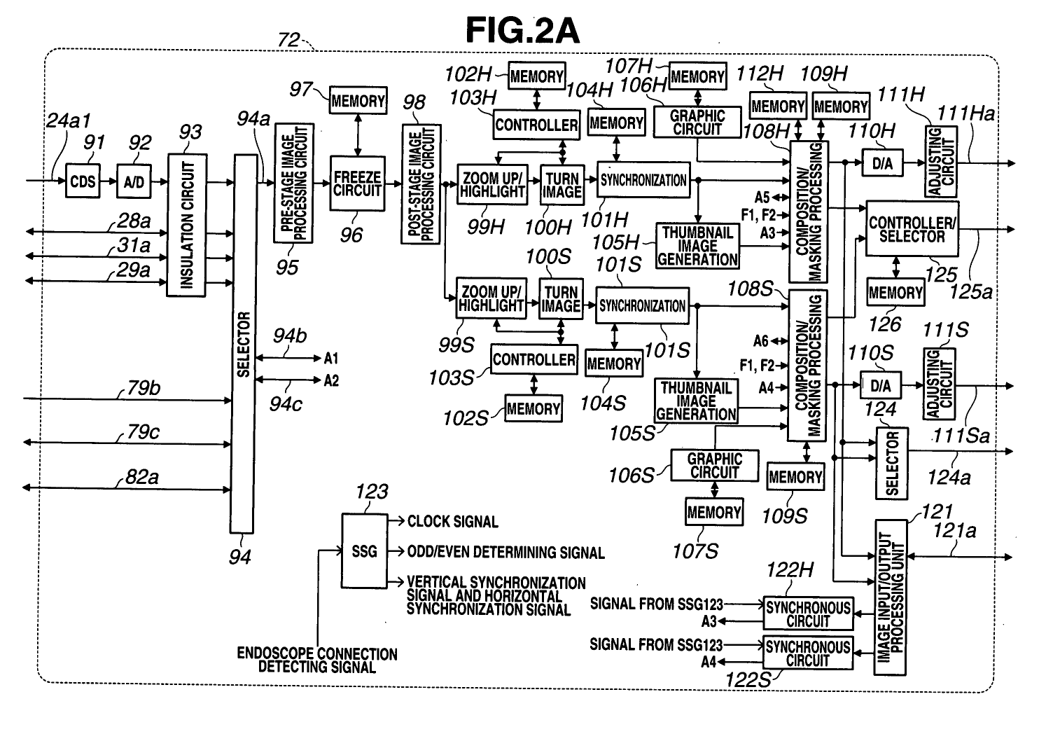

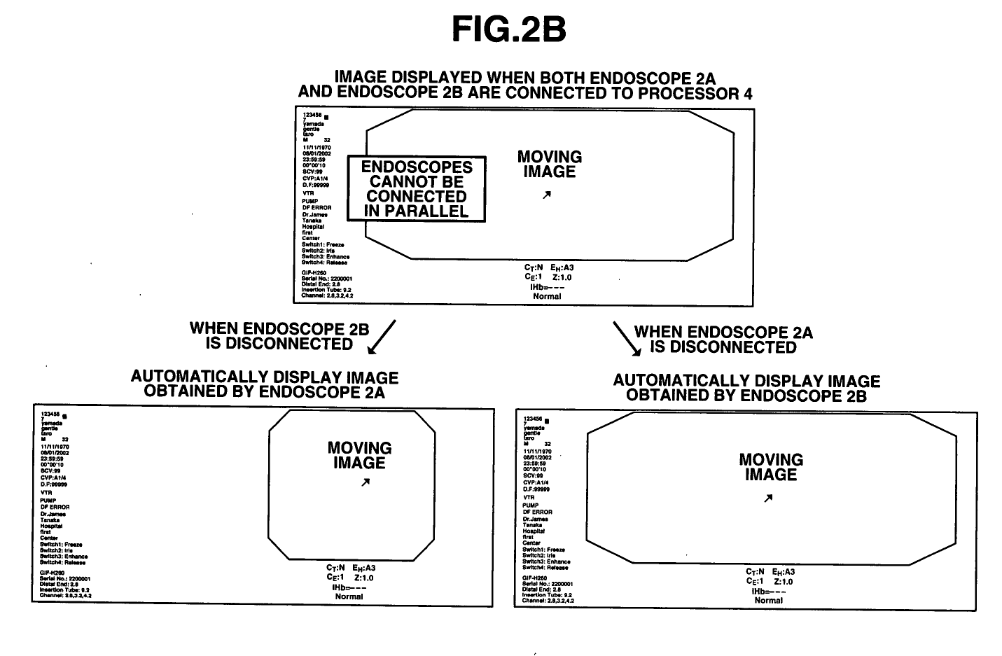

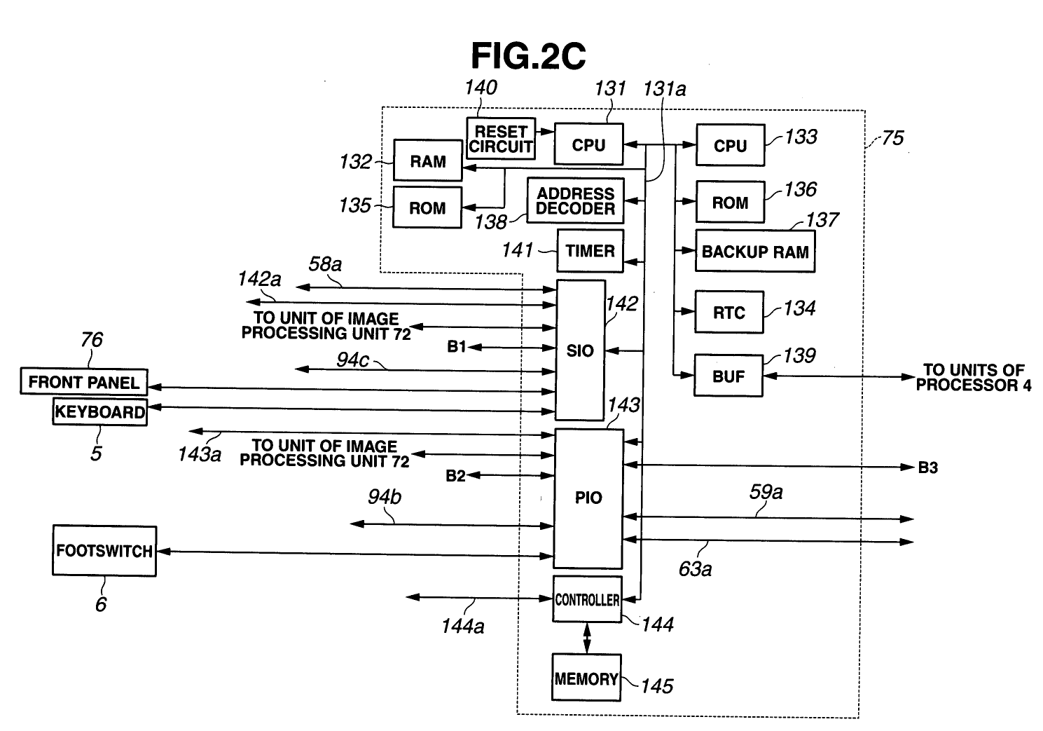

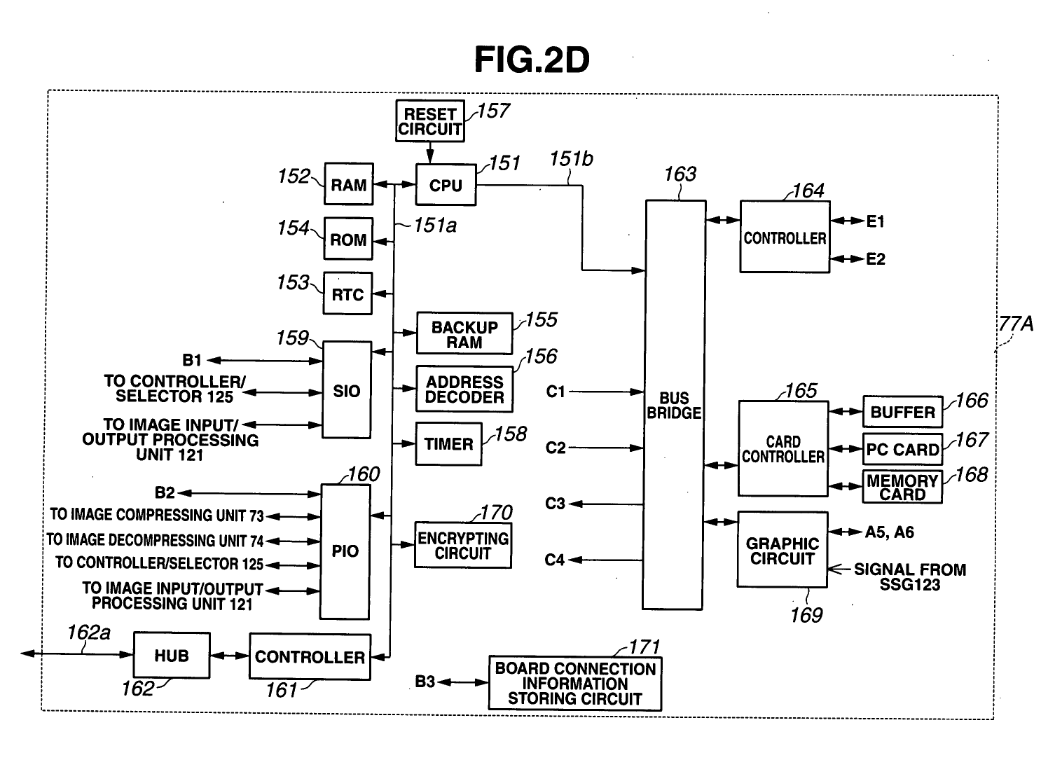

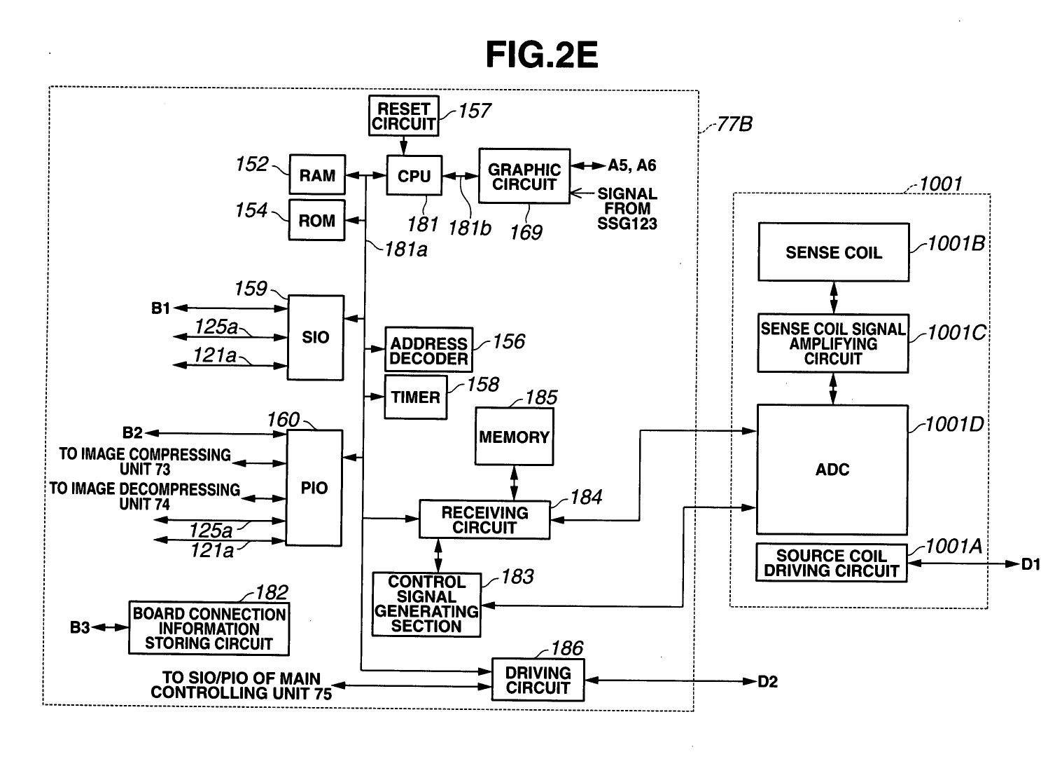

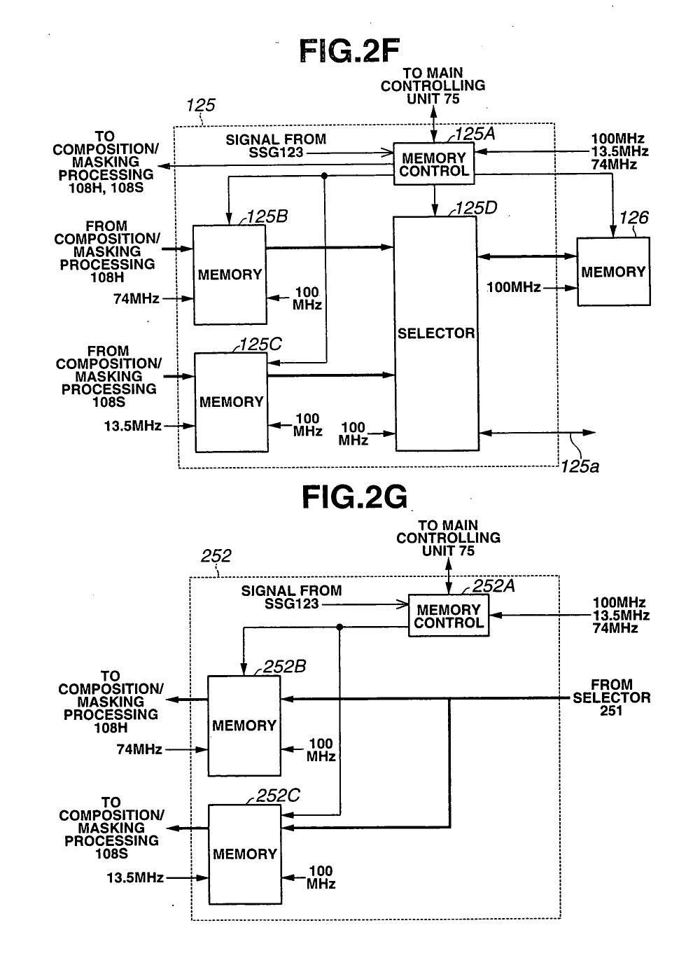

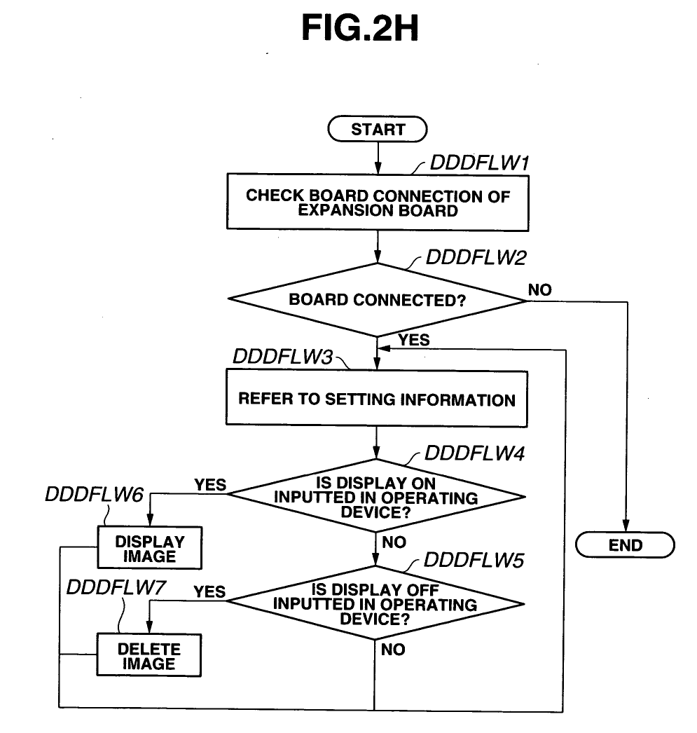

FIG. 1A is a diagram showing an example of a configuration of a main part of an endoscope system of the present embodiment. FIG. 1B is a diagram showing an example of a configuration of an endoscope in the endoscope system shown in FIG. 1A. FIG. 1C is a diagram showing an example of a configuration of an endoscope in the endoscope system shown in FIG. 1A different from the endoscope shown in FIG. 1B. FIG. 1D is a diagram showing an example of a configuration of light equipment in the endoscope system shown in FIG. 1A. FIG. 1E is a diagram showing an example of a configuration of a processor in the endoscope system shown in FIG. 1A. FIG. 2A is a diagram showing an example of a configuration of an image processing unit in a processor shown in FIG. 1E. FIG. 2B is a diagram showing an example of a screen that is displayed when both the endoscope shown in FIG. 1B and the endoscope shown in FIG. 1C are connected to the processor shown in FIG. 1E. FIG. 2C is a diagram showing an example of a configuration of a main controlling unit in the processor shown in FIG. 1E. FIG. 2D is a diagram showing an example of a configuration of an expansion controlling units connected to the processor shown in FIG. 1E. FIG. 2E is a diagram showing an example of a configuration of an expansion controlling unit different from the expansion controlling unit shown in FIG. 2D connected to the processor shown in FIG. 1E. FIG. 2F is a diagram showing an example of a configuration of a controller/selector in the processor shown in FIG. 1E. FIG. 2G is a diagram showing an example of a configuration of a synchronous circuit in an image decompressing unit of the processor shown in FIG. 1E. FIG. 2H is a flowchart showing an example of processing that the main controlling unit shown in FIG. 2C performs to detect (and when the main controlling unit shown in FIG. 2C detected) connection of the expansion controlling unit.

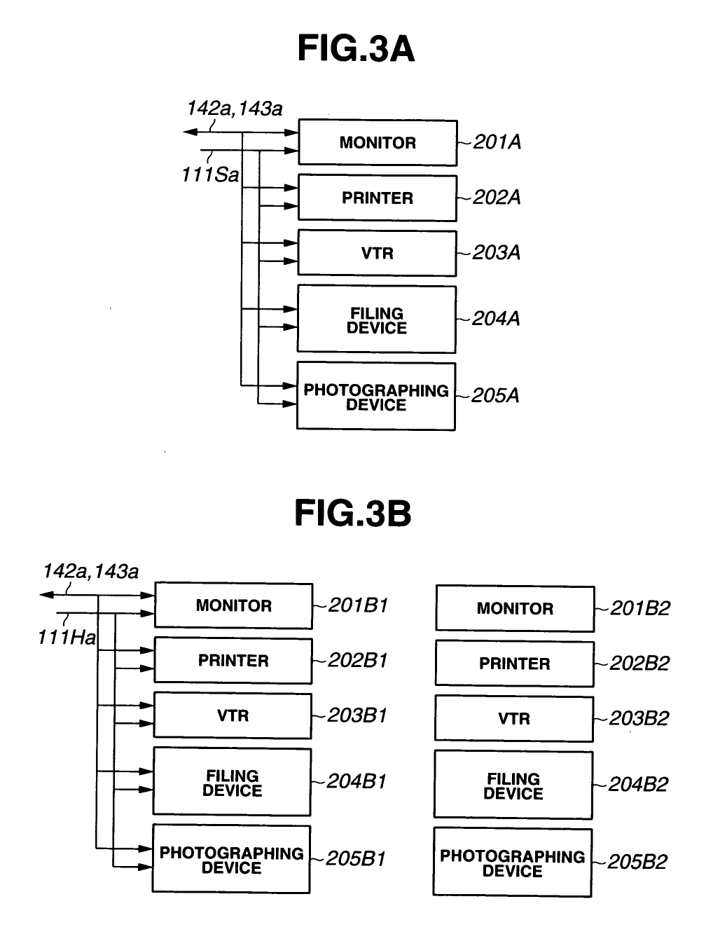

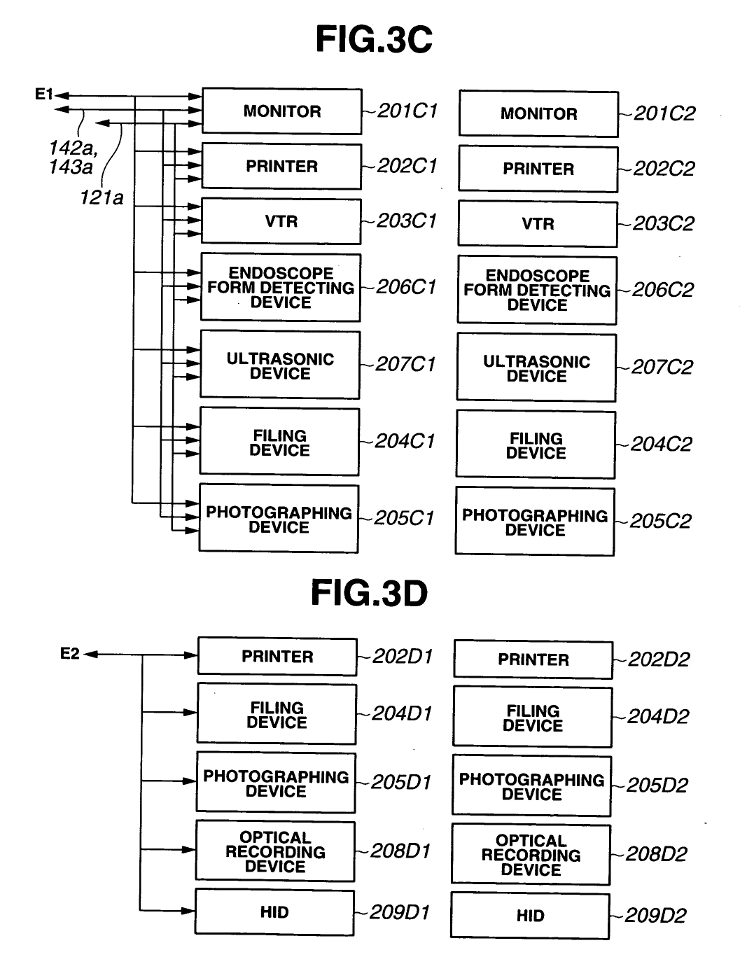

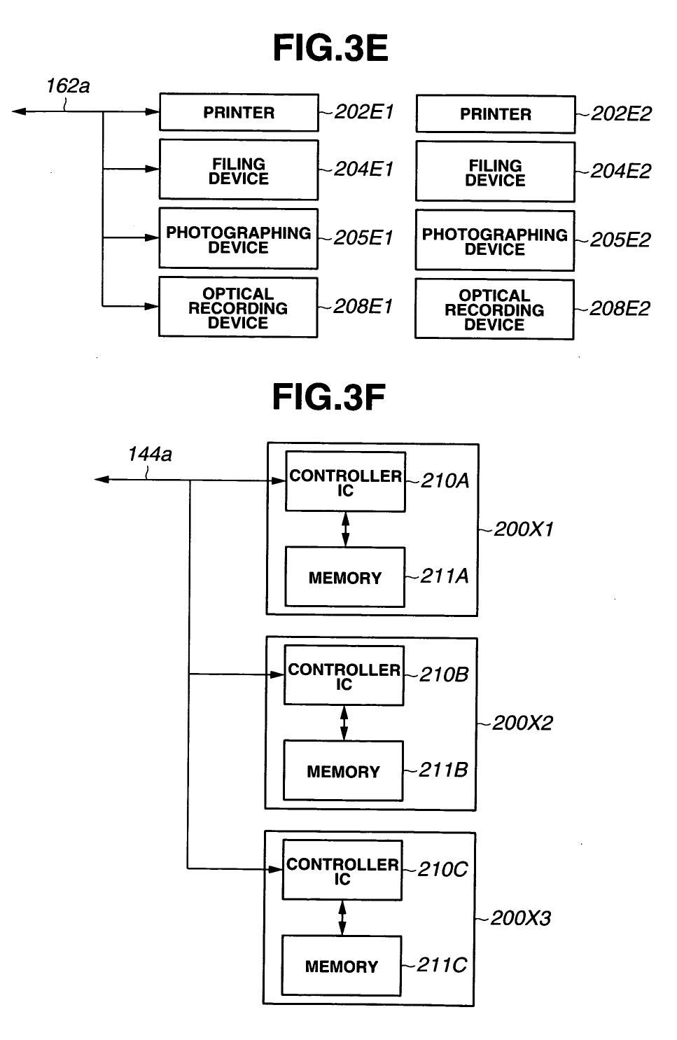

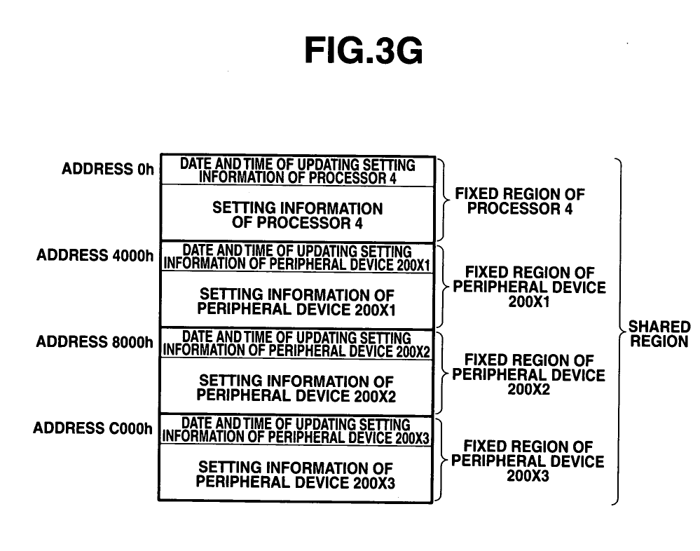

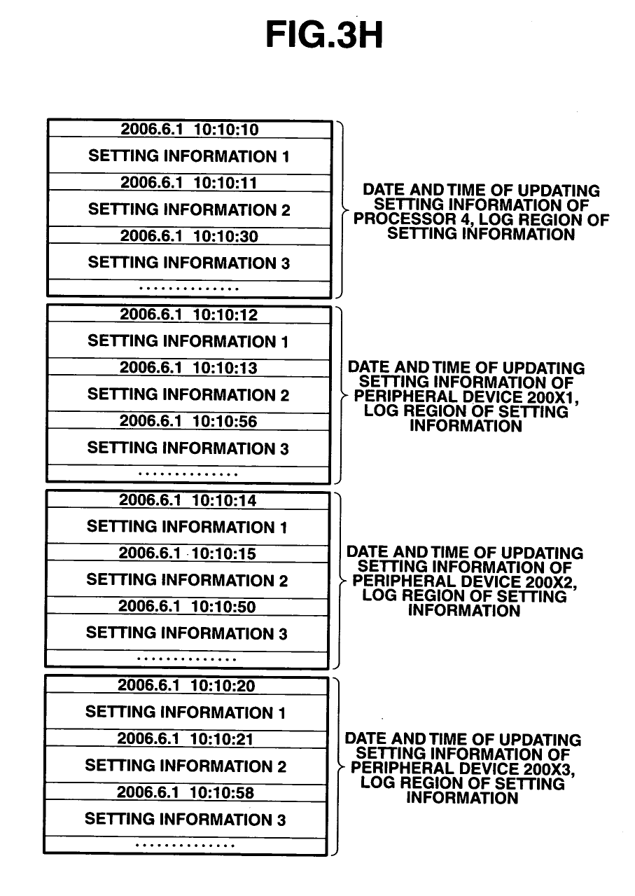

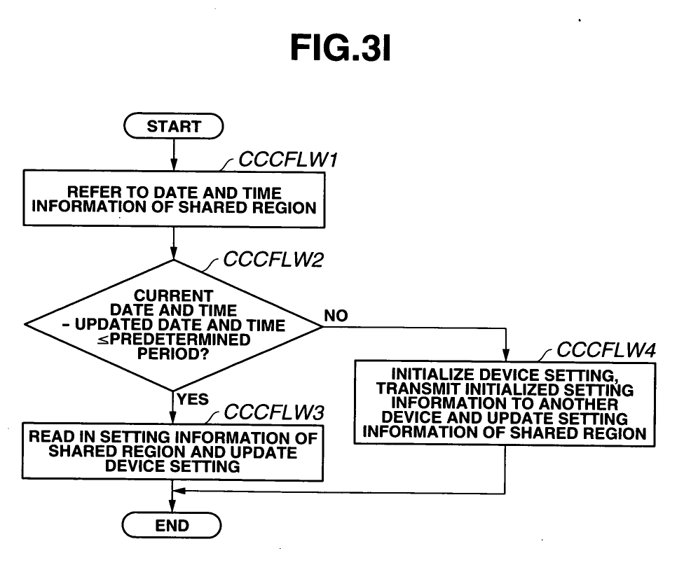

FIG. 3A is a diagram showing an example of a peripheral device that can be connected to the processor shown in FIG. 1E. FIG. 3B is a diagram showing an example of a peripheral device that can be connected to the processor shown in FIG. 1E, the example being different from the example shown in FIG. 3A. FIG. 3C is a diagram showing an example of a peripheral device that can be connected to the processor shown in FIG. 1E, the example being different from the examples shown in FIG. 3A and FIG. 3B. FIG. 3D is a diagram showing an example of a peripheral device that can be connected to the processor shown in FIG. 1E, the example being different from the examples shown in FIG. 3A, FIG. 3B and FIG. 3C. FIG. 3E is a diagram showing an example of a peripheral device that can be connected to the processor shown in FIG. 1E, the example being different from the examples shown in FIG. 3A, FIG. 3B, FIG. 3C and FIG. 3D. FIG. 3F is a diagram showing a simplified inner configuration of some of the peripheral devices shown in FIG. 3A to FIG. 3E. FIG. 3G is a diagram showing a configuration of data stored in a shared region of each of the memories shown in FIG. 3F. FIG. 3H is a diagram showing a configuration of data stored in a log region of each of the memories shown in FIG. 3F. FIG. 3I is a flowchart showing an example of processing related with the data stored in the shared regions shown in FIG. 3G.

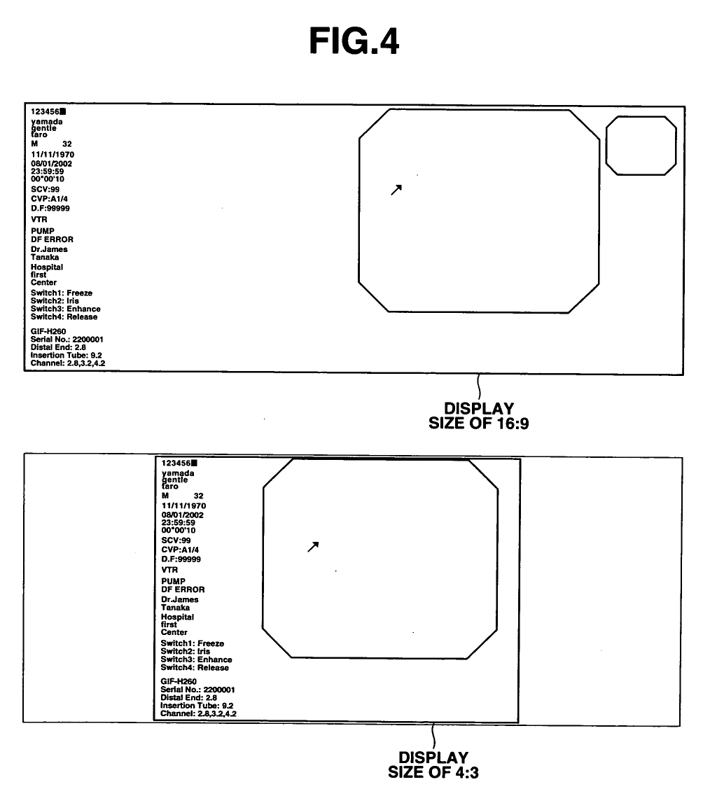

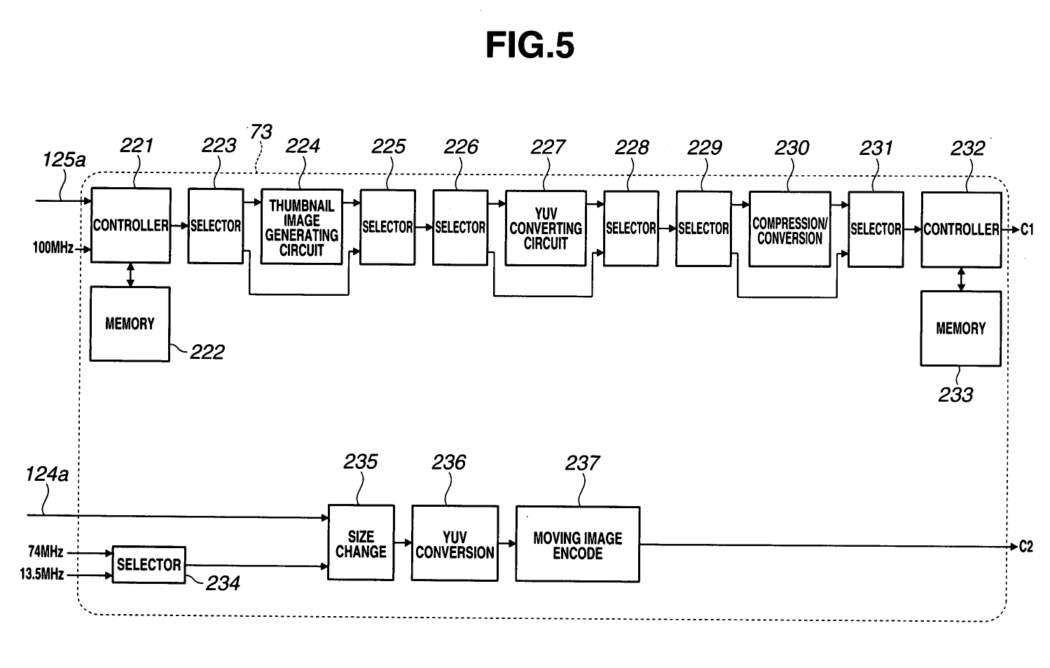

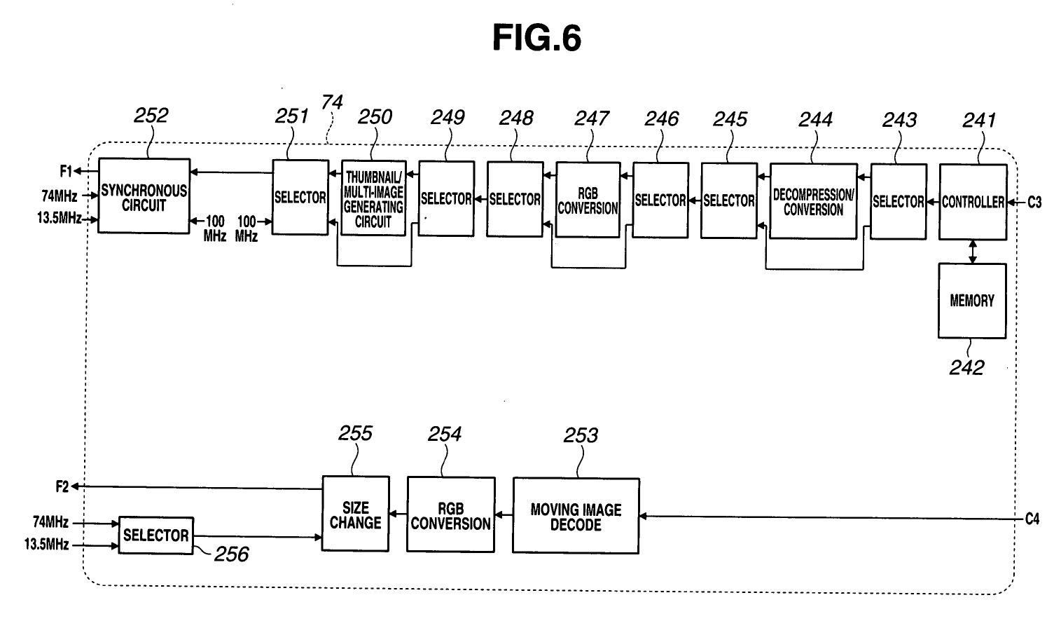

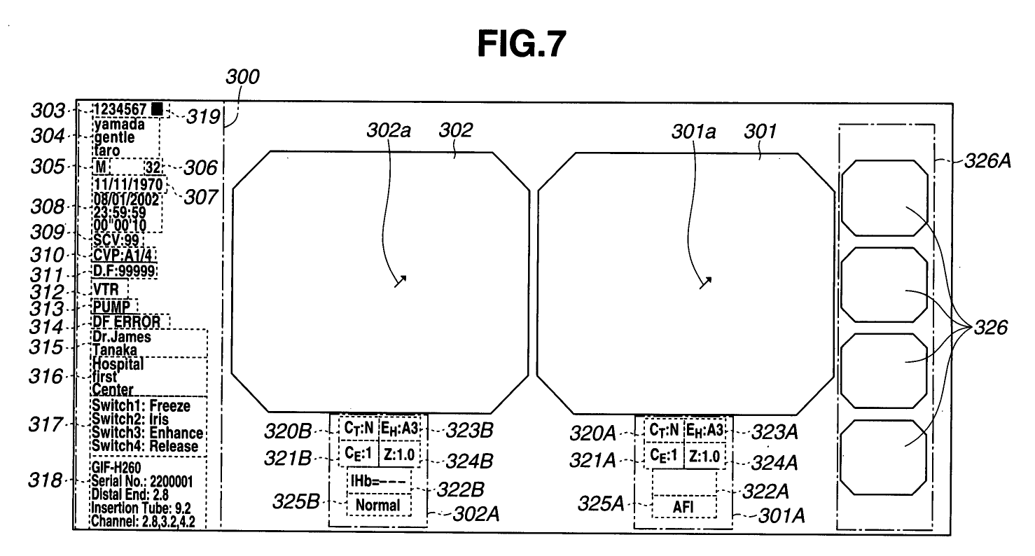

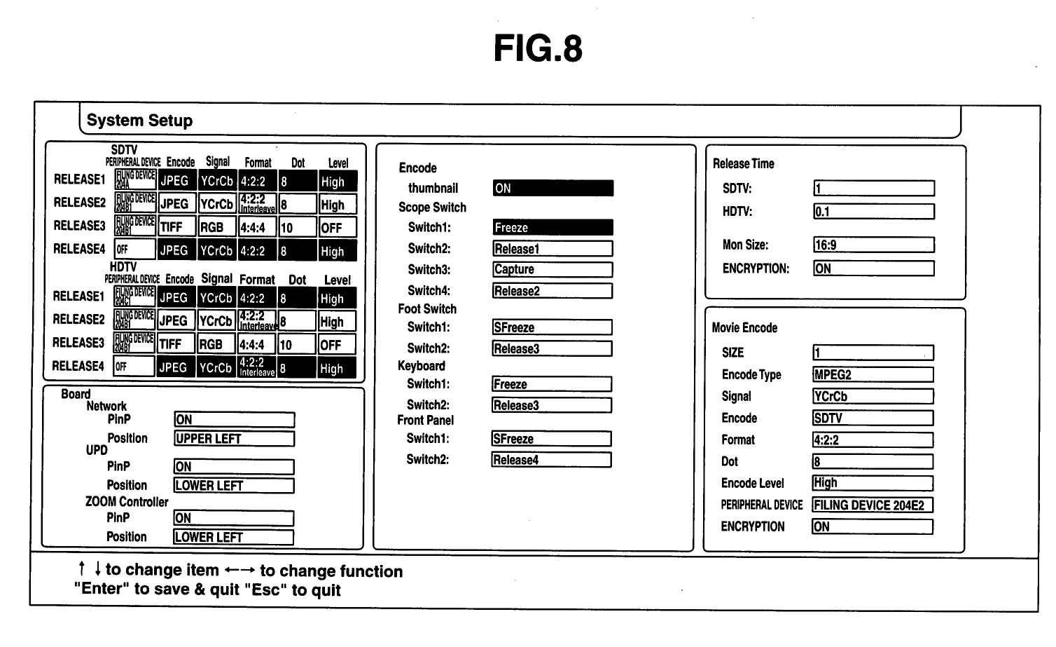

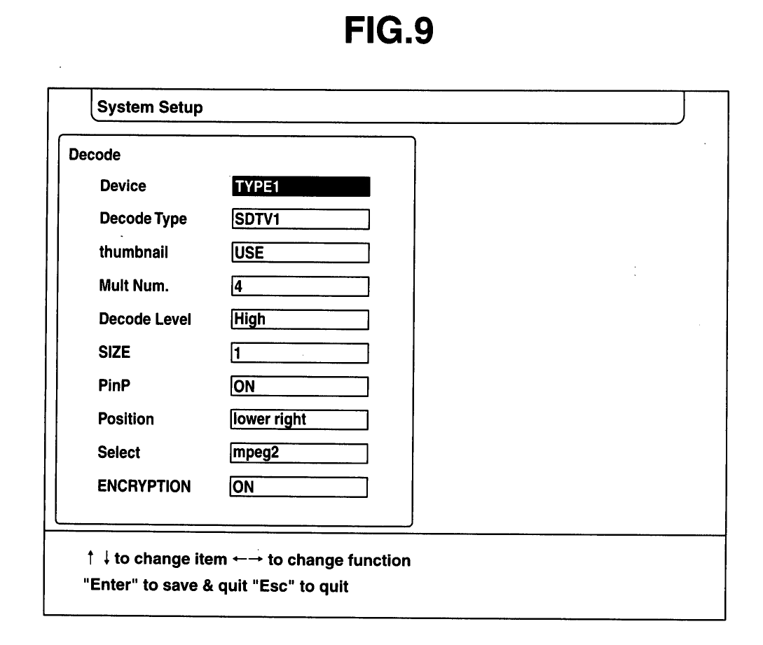

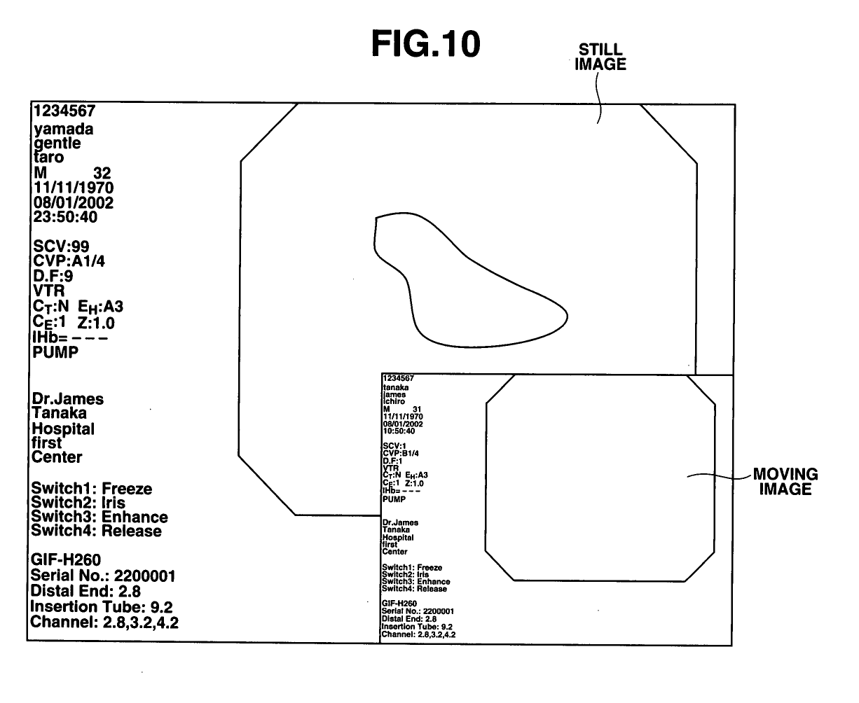

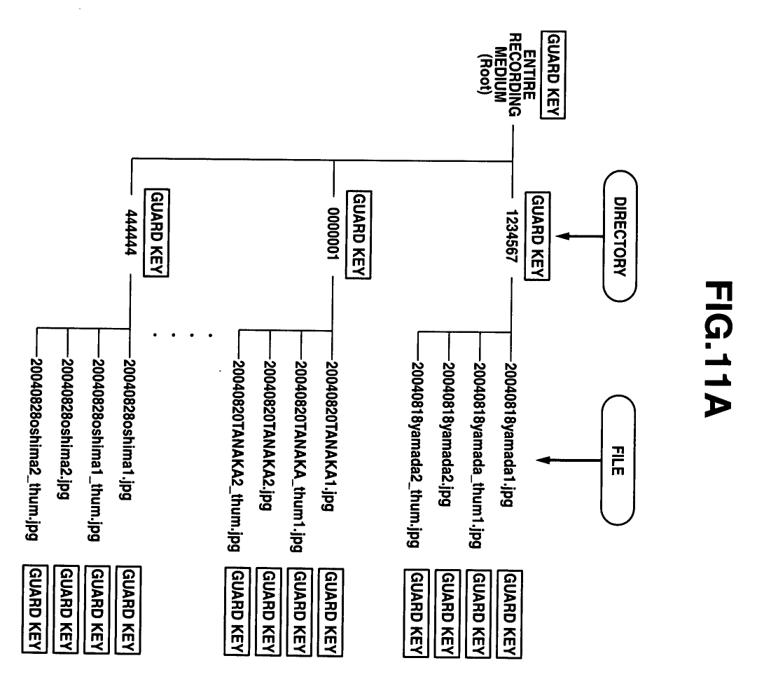

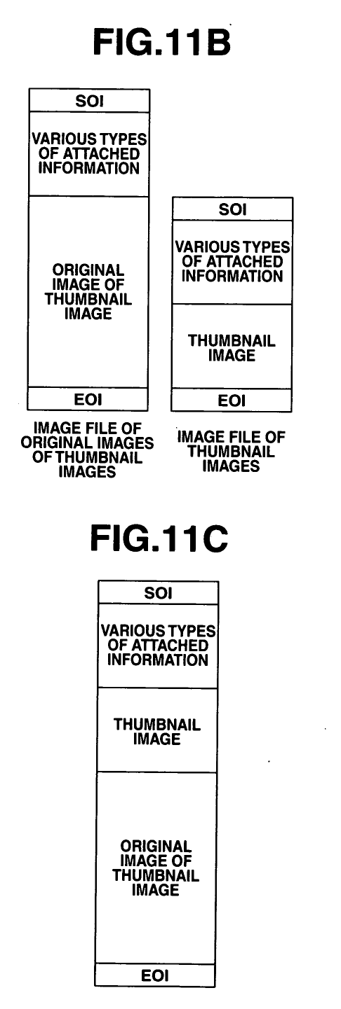

FIG. 4 is a diagram showing an example of display size (outputted size) of an image. FIG. 5 is a diagram showing an example of a configuration of an image compressing unit in the processor shown in FIG. 1E. FIG. 6 is a diagram showing an example of a configuration of an image decompressing unit in the processor shown in FIG. 1E. FIG. 7 is a diagram showing an example of an endoscope composite image generated by the image processing unit shown in FIG. 2A. FIG. 8 is a diagram showing an example of a setting screen of the processor shown in FIG. 1E. FIG. 9 is a diagram showing an example of another setting screen, which is among the setting screens of the processor shown in FIG. 1E, displayed after transition from the setting screen shown in FIG. 8. FIG. 10 is a diagram showing an example of a case where a moving image is displayed by PinP. FIG. 11A is a diagram showing an example of a directory structure used when an image is recorded in each filing device, each optical recording device and the like shown in FIG. 3A to FIG. 3E. FIG. 11B is a diagram showing an example of a data configuration of an image file of thumbnail images and an image file of images which originated the thumbnail images among files in the directory structures shown in FIG. 11A. FIG. 11C is a diagram showing another example of a data configuration of an image file of thumbnail images and an image file of images which originated the thumbnail images among files in the directory structures shown in FIG. 11A, the example being different from the example shown in FIG. 11B.

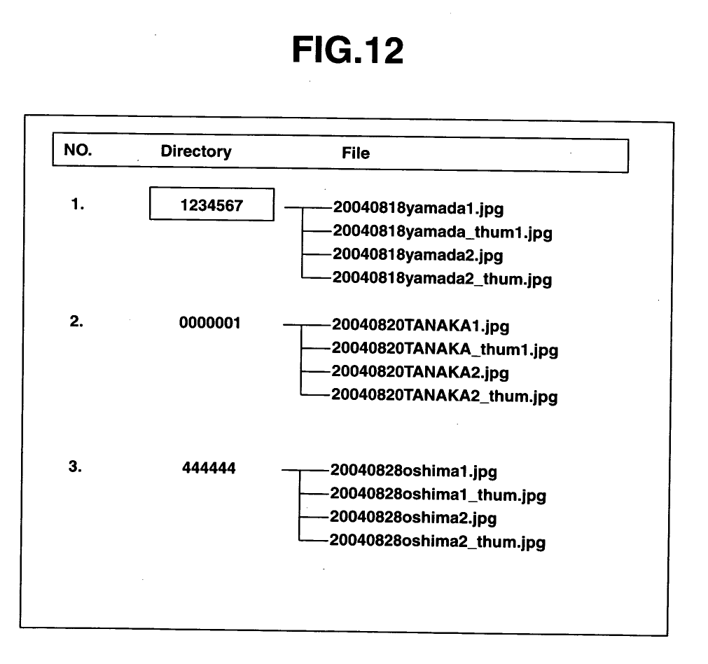

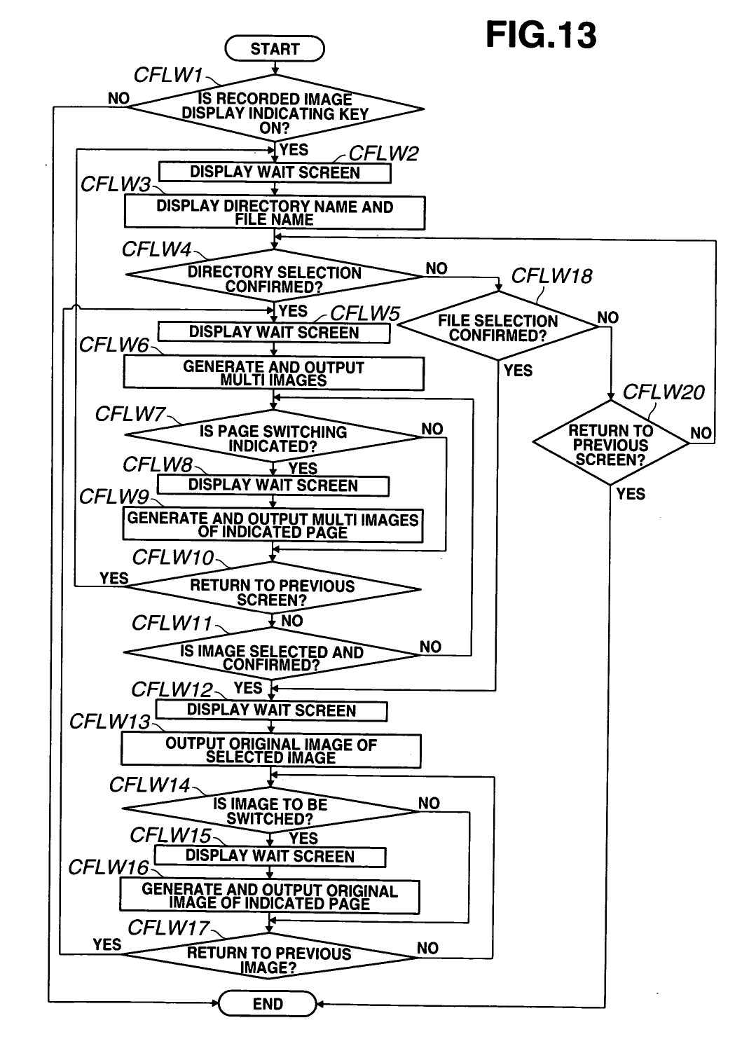

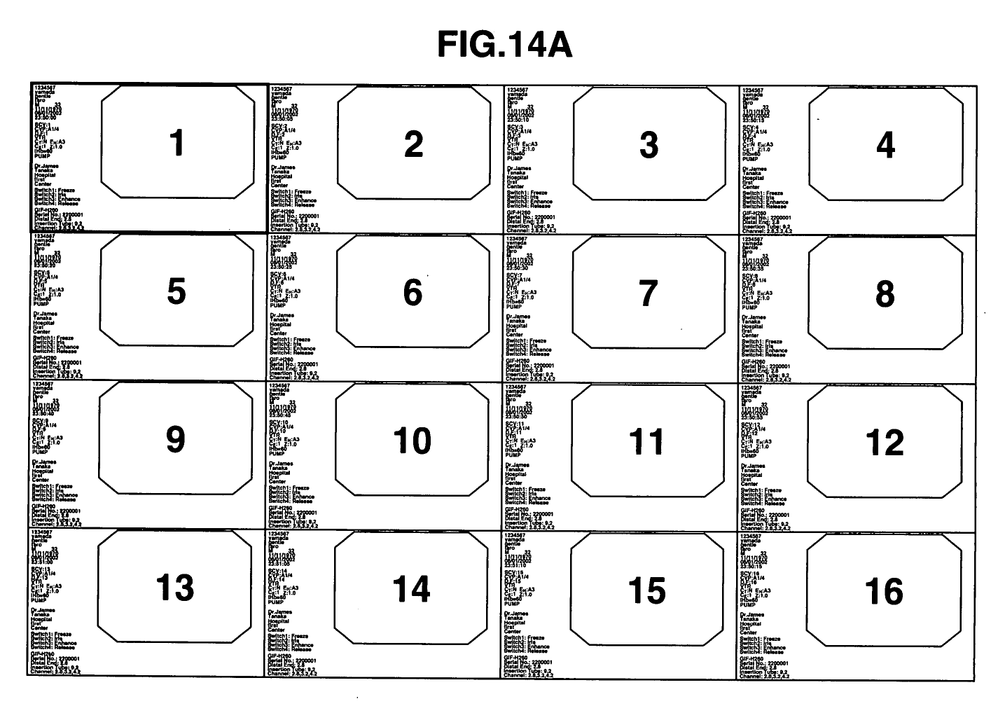

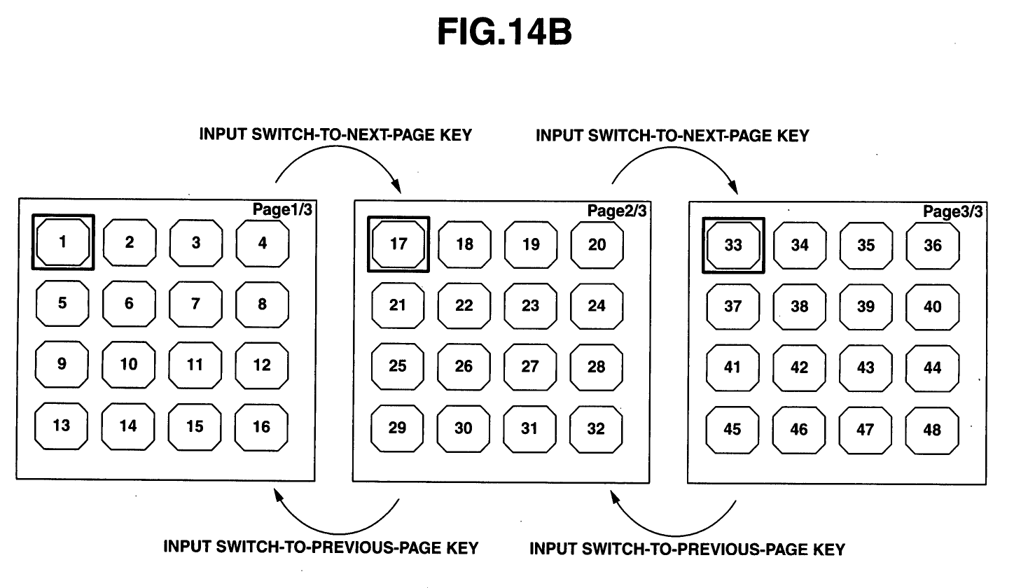

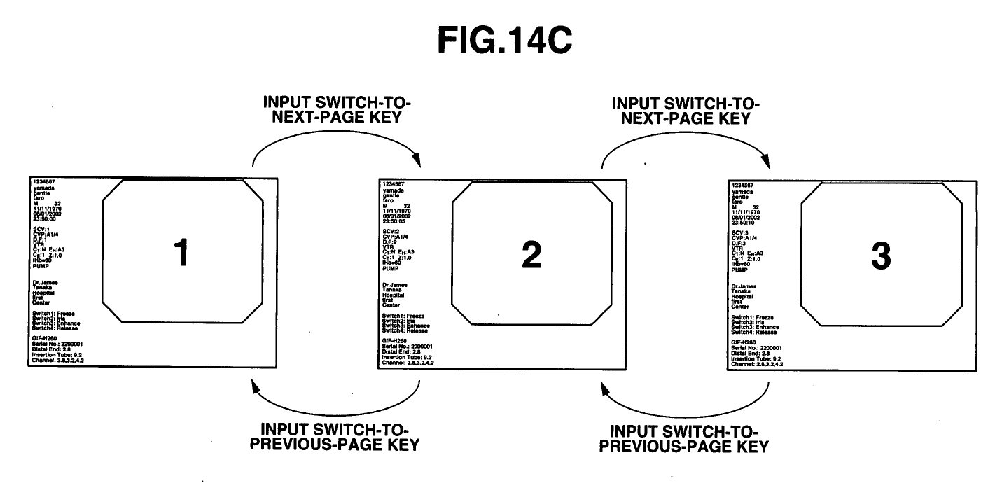

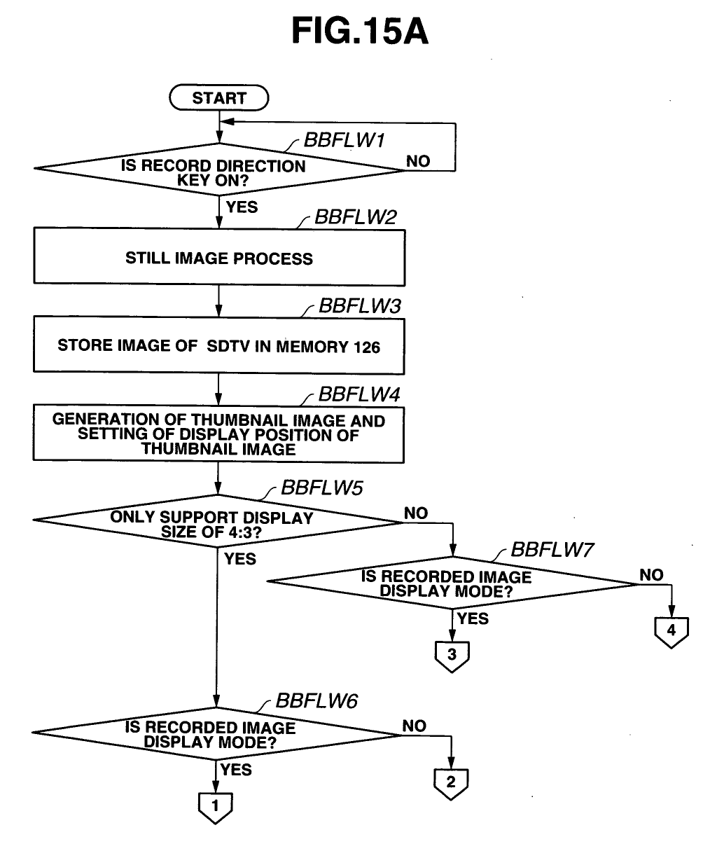

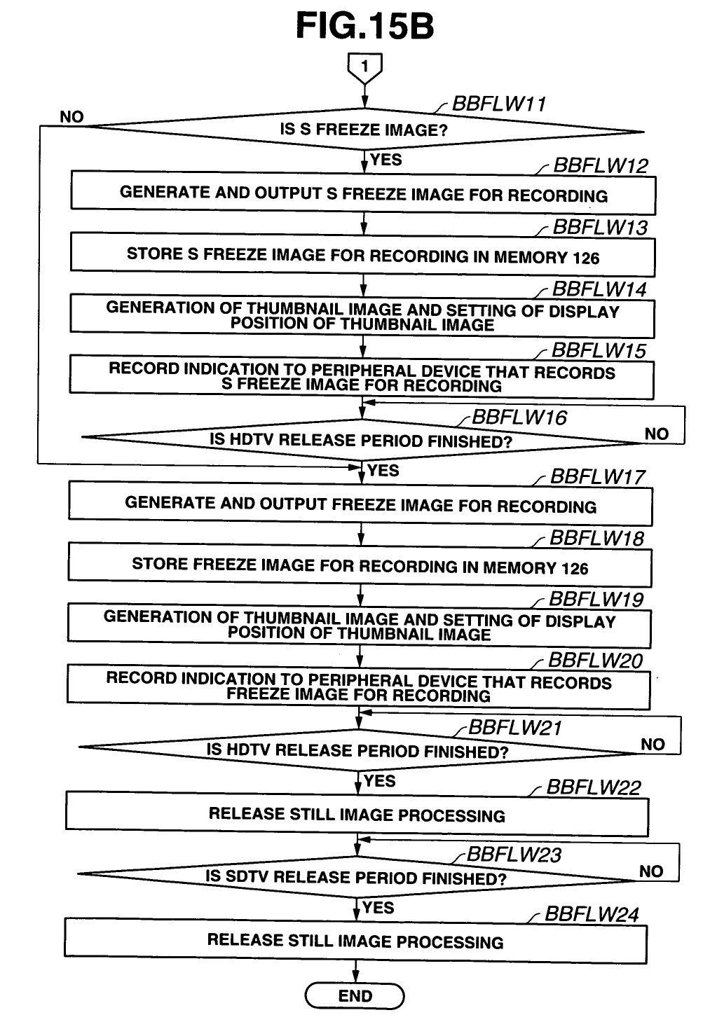

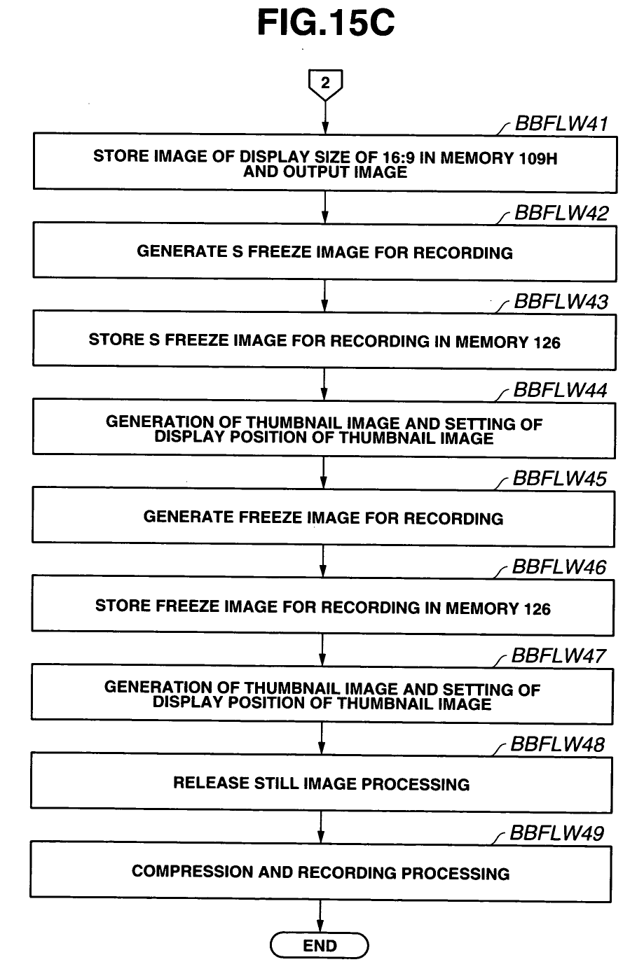

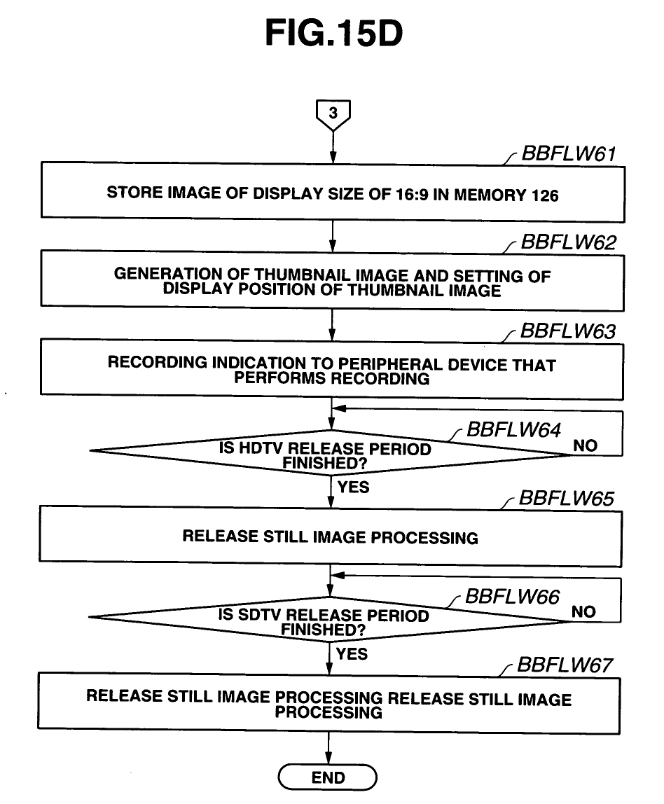

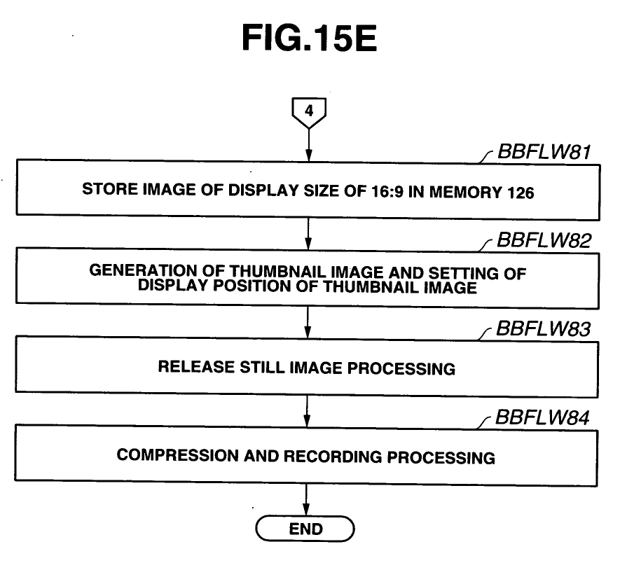

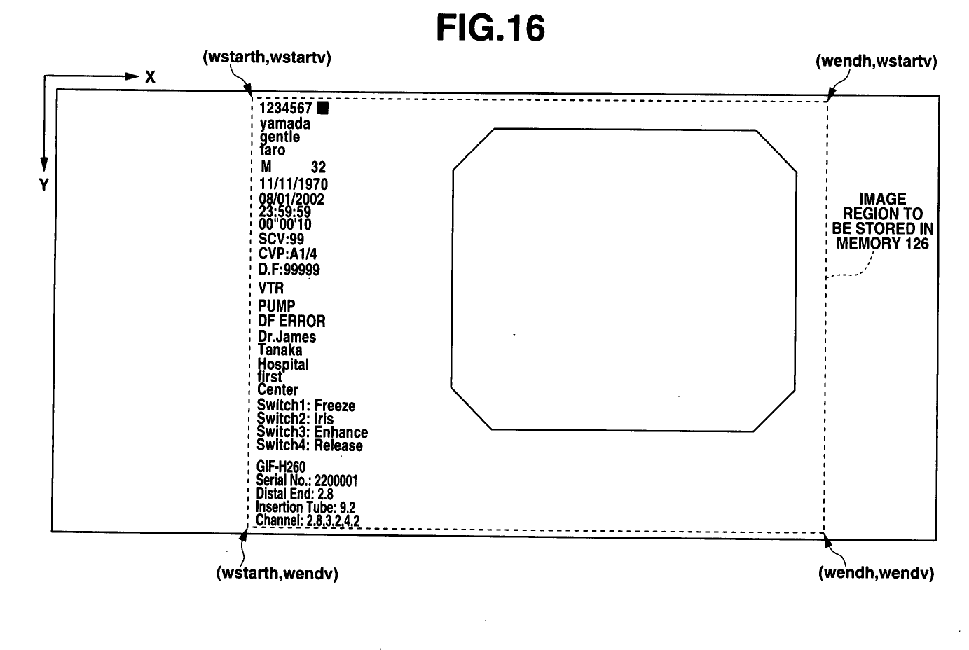

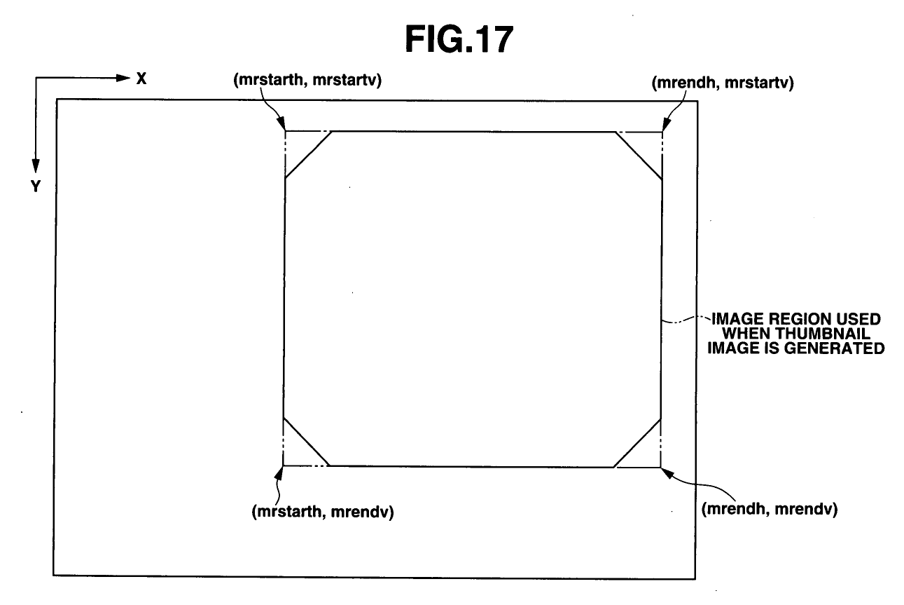

FIG. 12 is a diagram showing an example of a directory name and a file name displayed on a monitor and the like in a display form corresponding to the directory structure shown in FIG. 11A. FIG. 13 is a flowchart showing an example of control and processing performed by the main controlling unit shown in FIG. 2C when a still image recorded in the peripheral devices shown in FIG. 3A to FIG. 3E and the like is displayed. FIG. 14A is a diagram showing an example of multi-images generated by the processing shown in FIG. 13. FIG. 14B is a diagram showing an example of page switching when multi-images are generated by the processing shown in FIG. 13. FIG. 14C is a diagram showing an example of screen transition in the multi-images shown in FIG. 14A to display a selected image. FIG. 15A is a diagram showing an example of processing performed by the processor shown in FIG. 1E when record direction is performed. FIG. 15B is a diagram showing an example of processing performed by the processor shown in FIG. 1E following the processing shown in FIG. 15A when record direction is performed. FIG. 15C is a diagram showing an example different from the example shown in FIG. 15B of processing performed by the processor shown in FIG. 1E following the processing shown in FIG. 15A when record direction is performed. FIG. 15D is a diagram showing an example different from the examples shown in FIG. 15B and FIG. 15C of processing performed by the processor shown in FIG. 1E following the processing shown in FIG. 15A when record direction is performed. FIG. 15E is a diagram showing an example different from the examples shown in FIG. 15B, FIG. 15C and FIG. 15D of processing performed by the processor shown in FIG. 1E following the processing shown in FIG. 15A when record direction is performed. FIG. 16 is a diagram showing an example of a screen region that is outputted from a selector of the controller/selector shown in FIG. 2F to a memory and stored in the memory. FIG. 17 is a diagram showing an example of a thumbnail image generated by the thumbnail image generating section shown in FIG. 2A and a screen region that is taken as a standard for generating the thumbnail image.

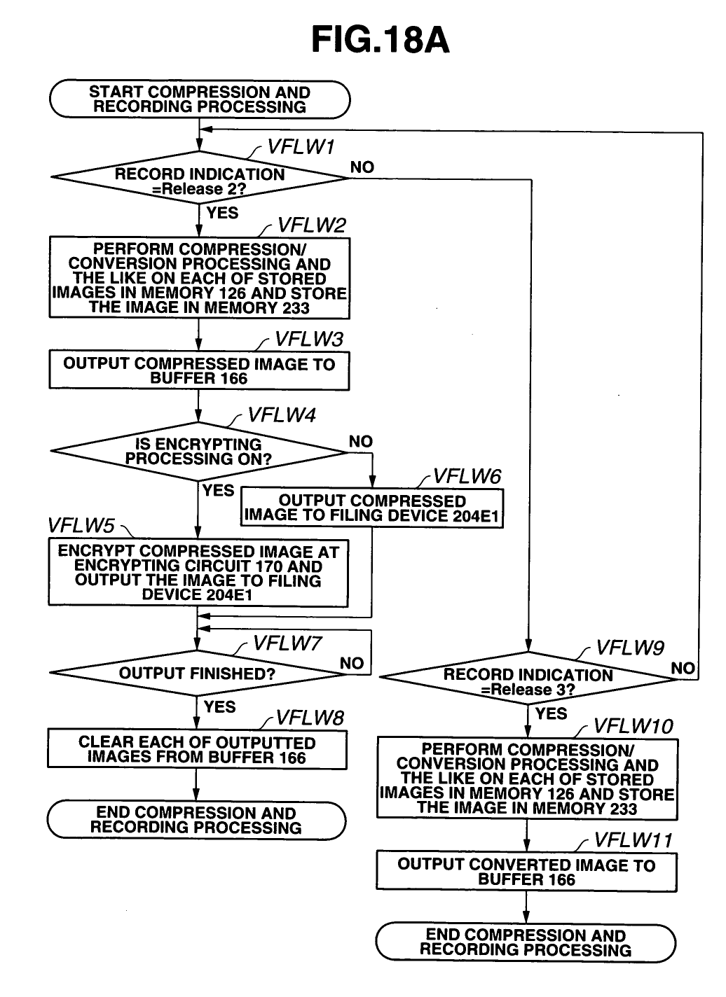

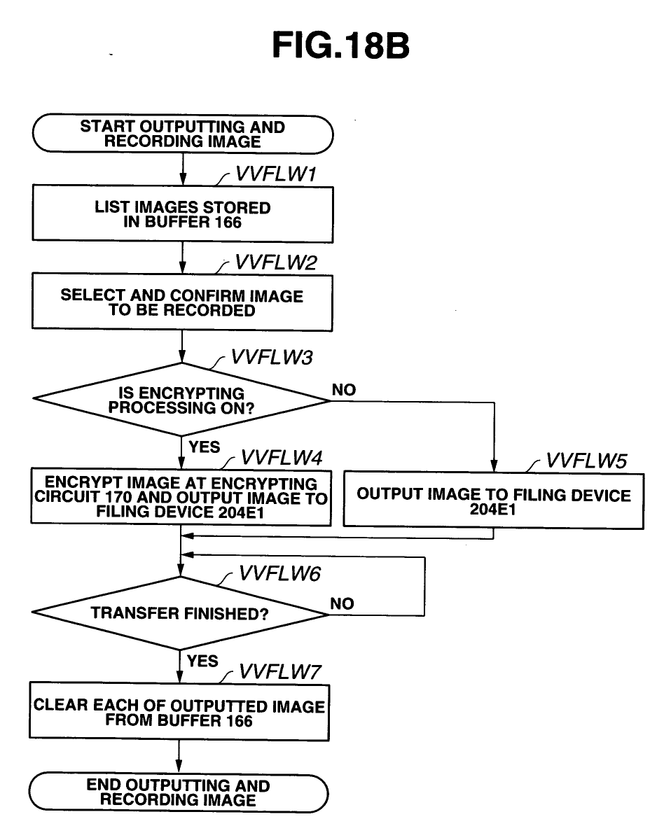

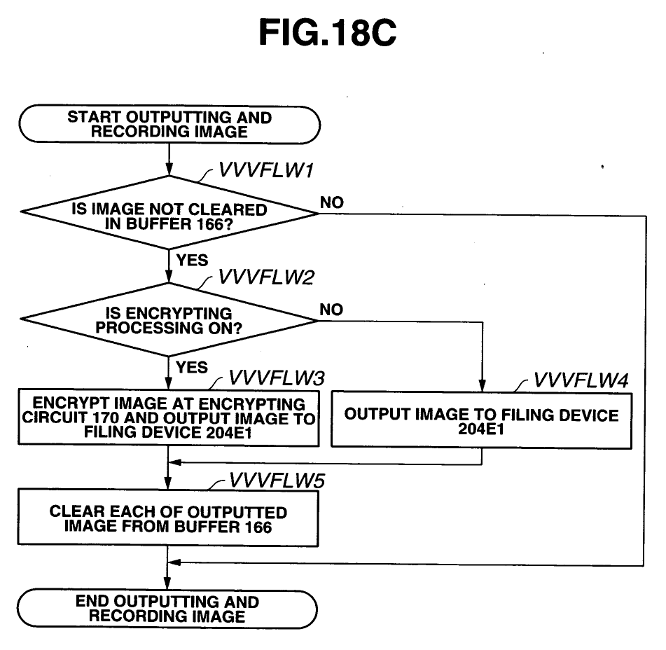

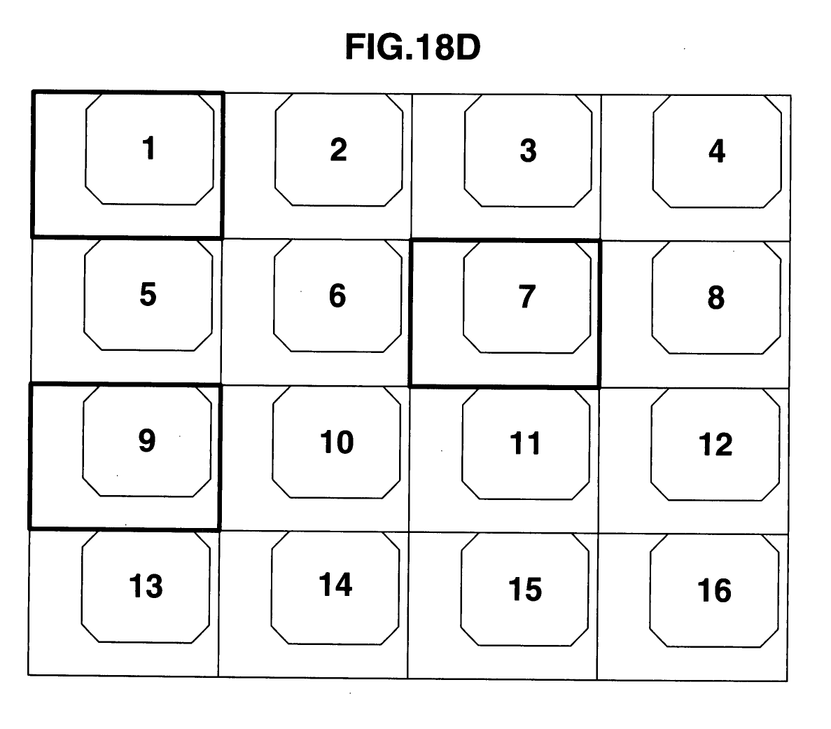

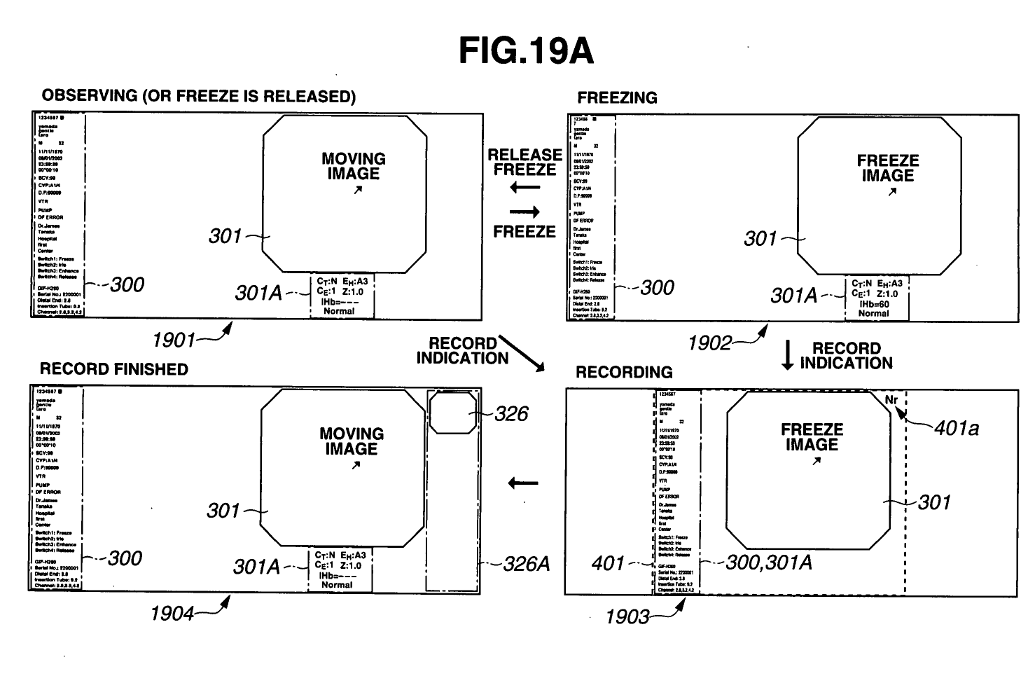

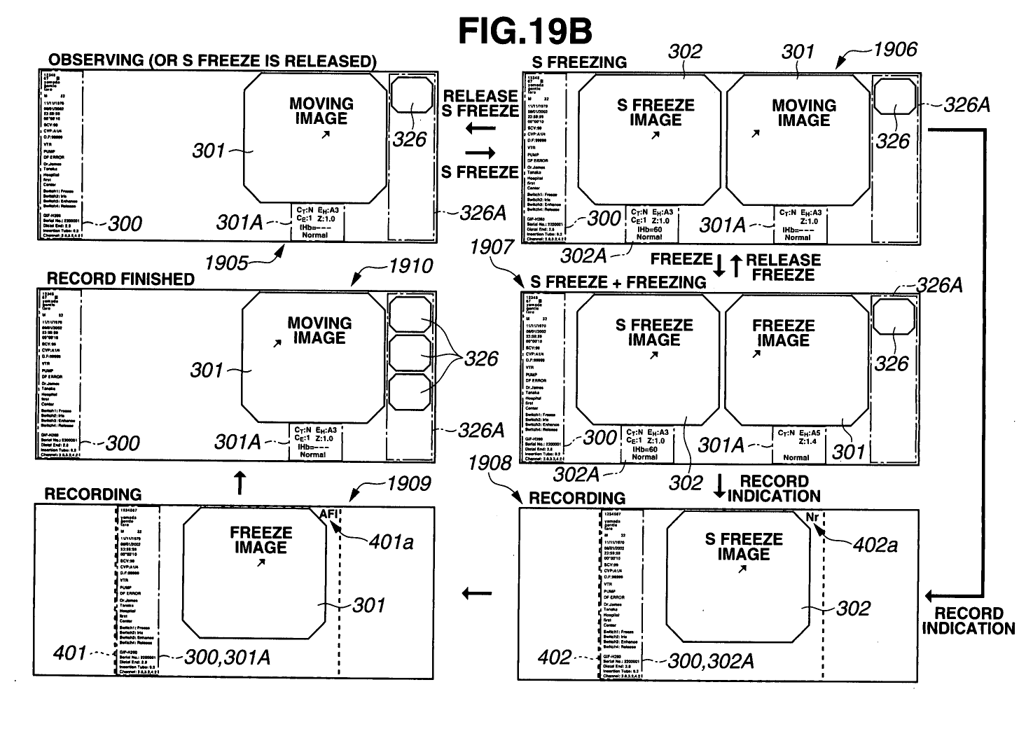

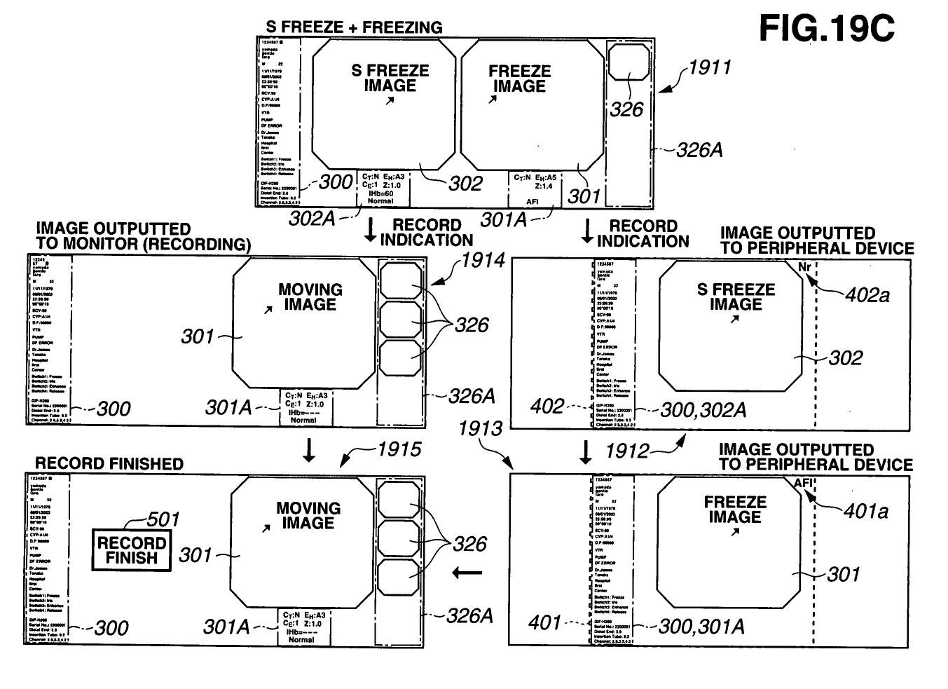

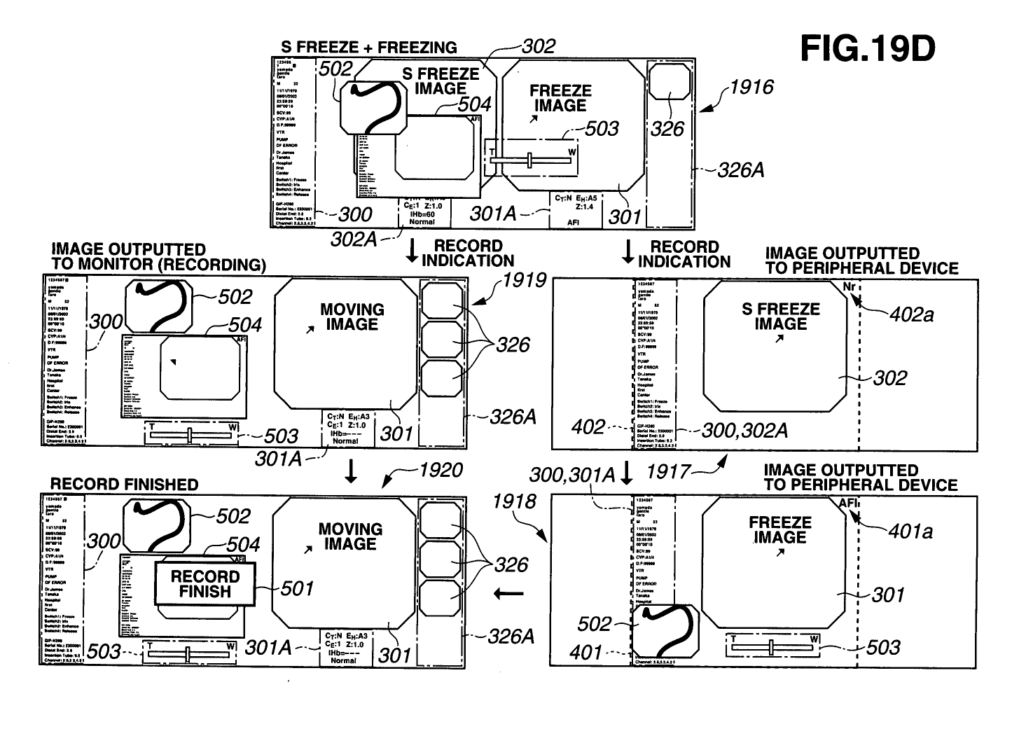

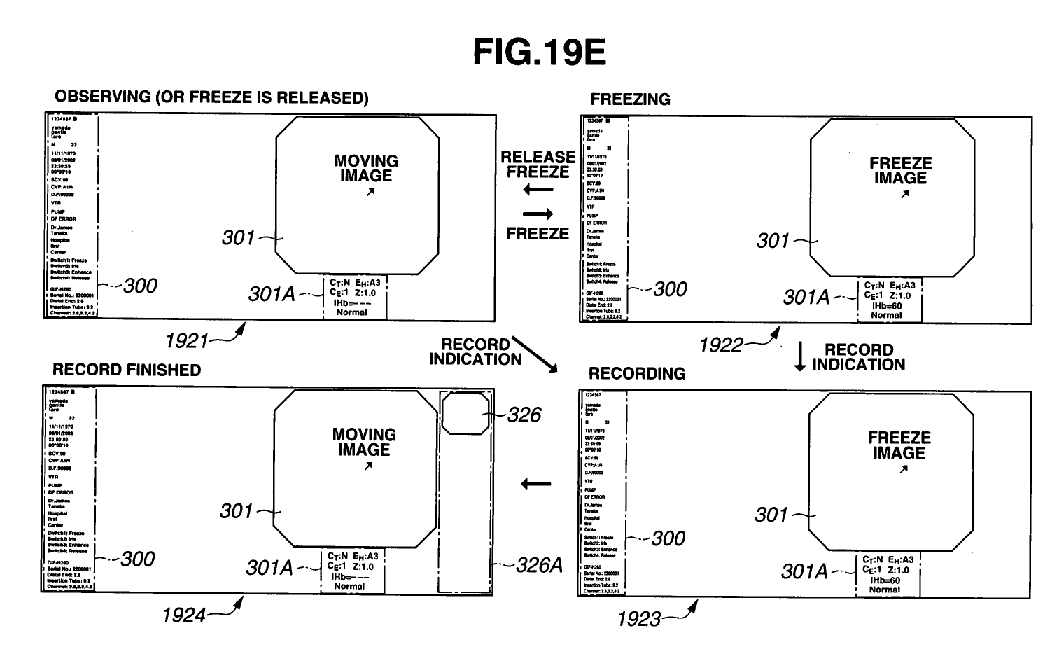

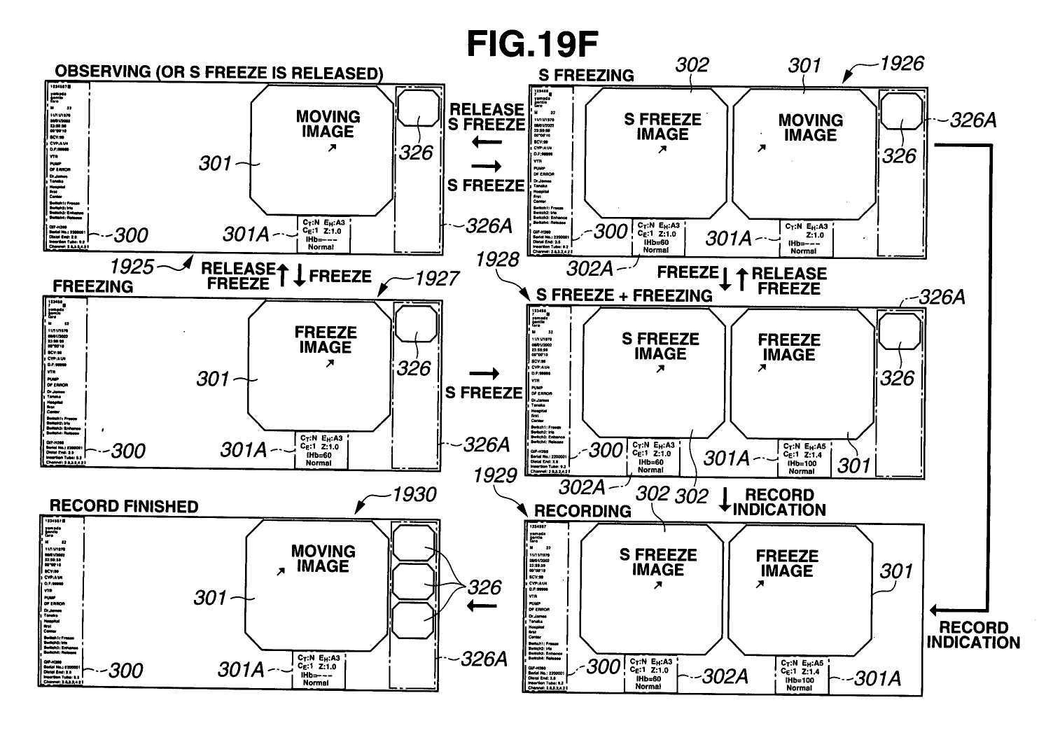

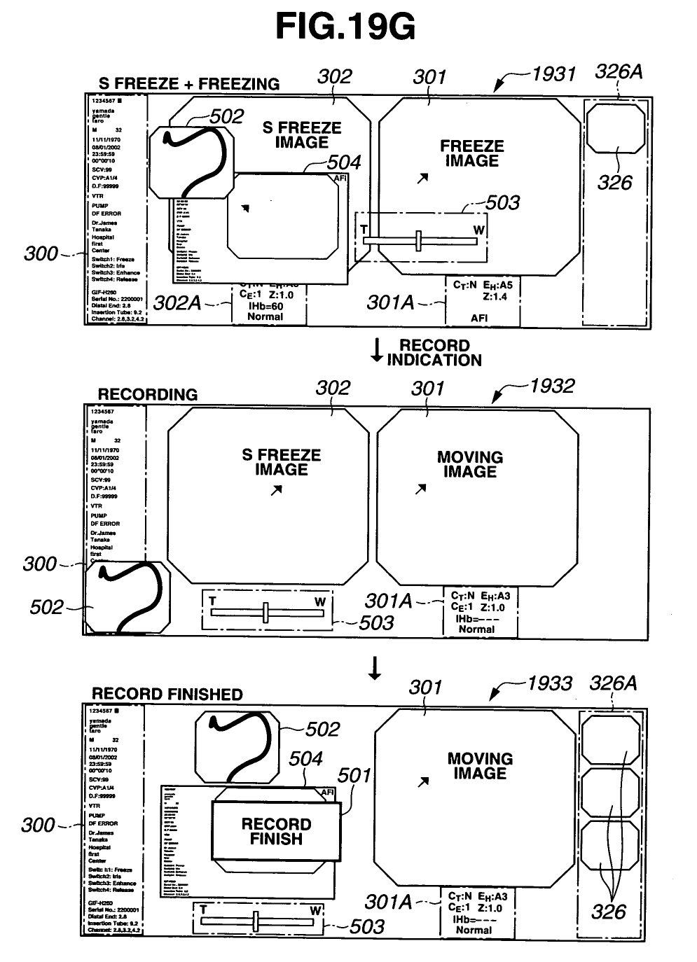

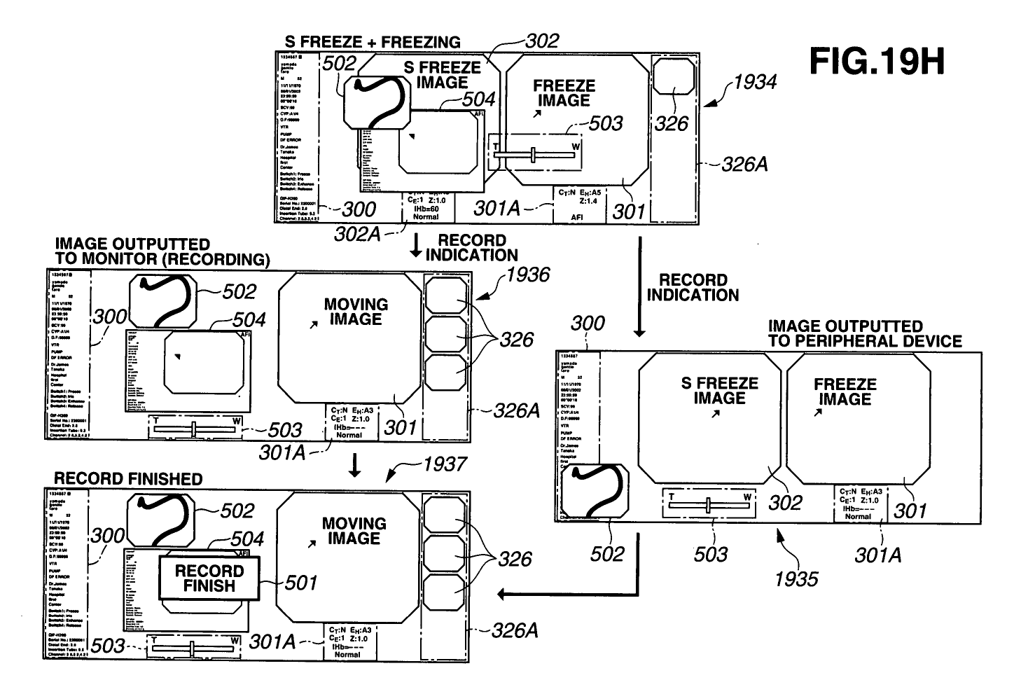

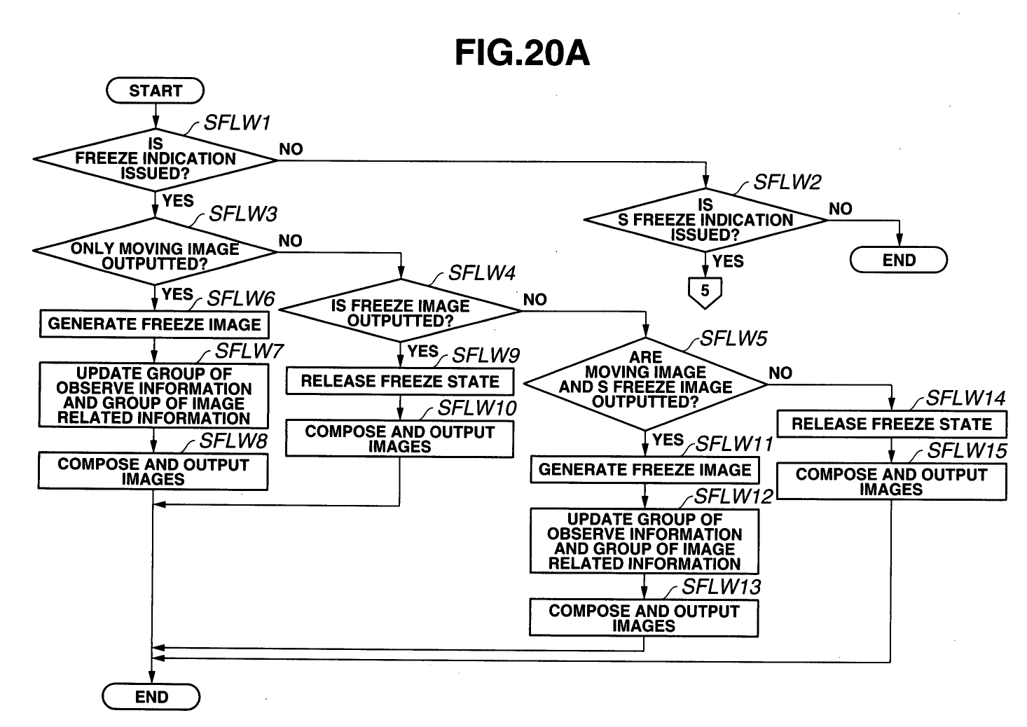

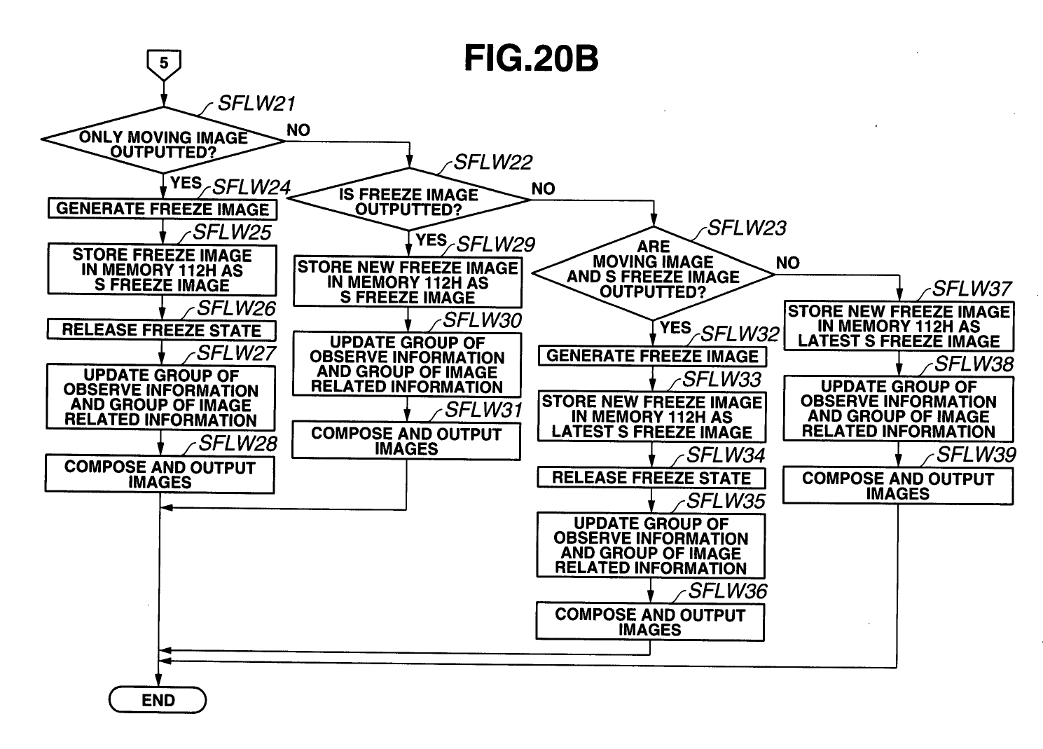

FIG. 18A is a flowchart showing an example of compression and recording included in the processing shown in FIG. 15B (FIG. 15C and FIG. 15D). FIG. 18B is a flowchart showing an example of processing performed when an image in a format of low compression rate, which is stored in a buffer by the processing shown in FIG. 18A, is recorded in a peripheral device and the like. FIG. 18C is a flowchart showing an example of processing performed when an image in a format of low compression rate, which is stored in a buffer by the processing shown in FIG. 18A, is recorded in a peripheral device and the like, different from the example shown in FIG. 18B. FIG. 18D is a diagram showing an example of multi-images which are generated for the purpose of selecting an image to be recorded from images stored in the buffer in the processing shown in FIG. 18B. FIG. 19A is a diagram showing an example of transition of screens displayed on a monitor or the like by the processing shown in FIG. 15B. FIG. 19B is a diagram showing an example of transition of screens displayed on a monitor or the like by the processing shown in FIG. 15B, different from the example shown in FIG. 19A. FIG. 19C is a diagram showing an example of transition of screens displayed on a monitor or the like by the processing shown in FIG. 15C. FIG. 19D is a diagram showing an example of transition of screens displayed on a monitor or the like by the processing shown in FIG. 15C, the example being different from the example shown in FIG. 19C. FIG. 19E is a diagram showing an example of transition of screens displayed on a monitor or the like by the processing shown in FIG. 15D. FIG. 19F is a diagram showing an example of transition of screens displayed on a monitor or the like by the processing shown in FIG. 15D, different from the example shown in FIG. 19E. FIG. 19G is a diagram showing an example of transition of screens displayed on a monitor or the like by the processing shown in FIG. 15D, different from the examples shown in FIG. 19E and FIG. 19F. FIG. 19H is a diagram showing an example of transition of screens displayed on a monitor or the like by the processing shown in FIG. 15E. FIG. 20A is a diagram showing an example of processing performed by each unit of the processor shown in FIG. 1E when freeze direction or S freeze direction is done. FIG. 20B is a diagram showing an example of processing performed by the processor shown in FIG. 1E following the processing shown in FIG. 20A when S freeze direction is done.

An endoscope system 1 includes endoscopes 2A and 2B which can be inserted in the body cavity of a patient for picking up an image of a subject in the body cavity, light equipment 3 for supplying illumination for illuminating the subject to the endoscopes 2A and 2B via a light guide cable 3a, and a processor 4 for performing control and the like on parts in the endoscope system 1 as a main part as shown in FIG. 1A. To the processor 4 as medical image processing apparatus, a keyboard 5 and a footswitch 6 are detachably (or integrally) connected as an operating device that can perform operating direction on each unit of the endoscope system 1. FIG. 1A shows a case where the light guide cable 3a is connected to the endoscope 2A.

As shown in FIG. 1B, the endoscope 2A has an insertion portion 21A that can be inserted in the body cavity of a patient, an objective optical system 22A that is provided at the distal end portion of the insertion portion 21A for imaging an image of a subject, an actuator 23A for moving the objective optical system 22A in the axial direction of the insertion portion 21A based on a driving signal outputted from an expansion board connected to the processor 4, a CCD (charge-coupled-device) 24A provided at an imaging position of the objective optical system 22A, and source coils 25A which are arranged almost over the insertion portion 21A for generating a magnetic field based on a driving signal outputted from an endoscope form detecting device to be described later.

The endoscope 2A has a light guide 26A for guiding illumination supplied from the light equipment 3 via the light guide cable 3a to the distal end portion of the insertion portion 21A, an operation section 27A for performing operational direction on the endoscope 2A and the like, an operation switching section 28A as an operating device including one or more switches provided for the operation section 27A, a connector 29A, a memory 30A for storing a program, endoscope specific information data and the like, a CPU 31A and a reset circuit 32A.

The endoscope 2A is detachably connected to the processor 4 via a connector 34A provided for the other end of a cable 33A extending from the connector 29A. The connector 29A outputs an endoscope connection detecting signal directing that the endoscope 2A is connected to the processor 4 via a signal line 29a to the processor 4. The signal line 29a is connected to the connector 29A by an end to be inserted through the cable 33A and connected to the inner circuit of the processor 4 by the other end.

The CCD 24A picks up an image of a subject that is imaged by the objective optical system 22A and outputs the picked up image of the subject to the processor 4 via the signal line 24a1 as an image pickup signal. The signal line 24a1 is connected to the CCD 24A by an end to be inserted through the cable 33A and connected to the inner circuit of the processor 4 by the other end. The CCD 24A is driven in accordance with a CCD driving signal generated at the processor 4 and inputted via a signal line 24a2. The signal line 24a2 is connected to the CCD 24A by an end to be inserted through the cable 33A and connected to the inner circuit of the processor 4 by the other end.

The memory 30A is a non-volatile memory, such as any of an EEPROM, a FLASH ROM, FRAM (registered trademark), an FeRAM, an MRAM, an OUM or an SRAM with a battery. The memory 30A stores the type of the CCD 24A, the type of the endoscope 2A, the serial number of the endoscope 2A, (one or more pieces of) white balance data, the number and the radius of forceps channels (not shown) of the endoscope 2A, the number of times of energizing the CPU 31A, the number of times each switch provided for the operation switching section 28A pressed, bending characteristics of the insertion portion 21A, the value of radius of the insertion portion 21A, the value of radius of the distal end portion of the insertion portion 21A, an zoom-up scale of the objective optical system 22A, forceps position information on the endoscope composite image, check direction information, the first date of usage of the endoscope 2A, the number of checking times, service information, manufacturer's comments, service comments, repair records, checking records, comment information, the program version of the CPU 31A, rental information, the number of the source coils 25A, the driving current for the source coils 25A, the driving voltage for the source coils 25A, information on whether the endoscope 2A is for direct-looking or side-looking and the like as the abovementioned endoscope specific information data.

The CPU 31A includes an interface circuit (a serial interface circuit or a parallel interface circuit), a watchdog timer, a timer, an SRAM, a FLASH ROM and the like, though they are not shown. The CPU 31A reads and writes various types of data stored in the memory 30A to and from the memory 30A via interface circuits (not shown).

The CPU 31A calculates the number of connections of the endoscope 2A, the number of times each switch provided for the operation switching section 28A is pressed, the number of times of energizing the CPU 31A, and the like.

The CPU 31A exchanges the result calculated by the CPU 31A and various types of data stored in the memory 30A with the processor 4 via a signal line 31a. The signal line 31a is connected to the CPU 31A by an end to be inserted through the cable 33A and connected to the inner circuit of the processor 4 by the other end.

The reset circuit 32A performs reset according to the timing of a power source supplied from the processor 4 being changed and the timing based on the watchdog timer in the CPU 31A.

A switch ON/OFF signal generated in response to operation of each switch of the operation switching section 28A and an endoscope connection detecting signal generated at the connector 29A are outputted to the processor 4 via a signal line 28a. The signal line 28a is connected to each switch of the operation switching section 28A by an end to be inserted through the cable 33A and connected to the inner circuit of the processor 4 by the other end. Here, the switch ON/OFF signal generated in response to operation of each switch of the operation switching section 28A and an endoscope connection detecting signal generated at the connector 29A are generated with the driving voltage supplied from a driving circuit 71 of the processor 4.

As shown in FIG. 1C, the endoscope 2B has an insertion portion 21B that can be inserted in the body cavity of a patient, an objective optical system 22B that is provided at the distal end portion of the insertion portion 21B for imaging an image of a subject, an actuator 23B for moving the objective optical system 22B in the axial direction of the insertion portion 21B based on a driving signal outputted from an expansion board connected to the processor 4, a CCD (charge-coupled-device) 24B provided at an imaging position of the objective optical system 22B, and source coils 25B which are arranged almost over the insertion portion 21B for generating a magnetic field based on a driving signal outputted from an endoscope form detecting device to be described later.

The endoscope 2B has a light guide 26B for guiding illumination supplied from the light equipment 3 via the light guide cable 3a to the distal end portion of the insertion portion 21B, an operation section 27B for performing operational direction on the endoscope 2B and the like, an operation switching section 28B as an operating device including one or more switches provided for the operation section 27B, a connector 29B, a memory 30B for storing a program, endoscope specific information data and the like, a CPU 31B and a reset circuit 32B.

The endoscope 2B is detachably connected to the processor 4 via a connector 34B provided for the other end of a cable 33B extending from the connector 29B.

The CCD 24B picks up an image of a subject that is imaged by the objective optical system 22B and outputs the picked up image of the subject to the CDS (correlation double sampling) circuit 35B via the signal line 24b1 as an image pickup signal.

When the endoscope 2B is connected to the processor 4, an endoscope connection detecting signal is outputted to the processor 4 via a P/S converting section (abbreviated as P/S in the specification hereinafter and the drawings) 37 and the like.

The CDS circuit 35B performs correlation double sampling on an image pickup signal outputted from the CCD 24B and outputs the image pickup signal through the correlation double sampling to an A/D converting section (abbreviated as A/D in the specification hereinafter and the drawings) 36B via a signal line 35b.

The A/D 36B converts an analog image pickup signal outputted from the CDS circuit 35B into a digital signal and then outputs the digital signal to a P/S 37B via a signal line 36b.

The memory 30B is a non-volatile memory, such as any of an EEPROM, a FLASH ROM, FRAM, an FeRAM, an MRAM, an OUM or an SRAM with a battery. The memory 30B stores the type of the CCD24B, the type of the endoscope 2B, the serial number of the endoscope 2B, (one or more pieces of) white balance data, the number and the radius of forceps channels (not shown) of the endoscope 2B, the number of times of energizing the CPU 31B, the number of times each switch provided for the operation switching section 28B being pressed, bending characteristics of the insertion portion 21B, the value of radius of the insertion portion 21B, the value of radius of the distal end portion of the insertion portion 21B, an zoom-up scale of the objective optical system 22B, forceps position information on the endoscope composite image, check direction information, the first used date of the endoscope 2B, the number of checking times, service information, manufacturer's comments, service comments, repair records, checking records, comment information, the program version of the CPU 31B, rental information, the number of the source coils 25B, the driving current for the source coils 25B, the driving voltage for the source coils 25B, information on whether the endoscope 2B is for direct-looking or side-looking and the like as the abovementioned endoscope specific information data.

The CPU 31B includes an interface circuit (a serial interface circuit or a parallel interface circuit), a watchdog timer, a timer, an SRAM, a FLASH ROM and the like, though they are not shown. The CPU 31B reads and writes various types of data stored in the memory 30B to and from the memory 30B via interface circuits (not shown).

The CPU 31B calculates the number of connections of the endoscope 2B, the number of times each switch provided for the operation switching section 28B is pressed, the number of times of energizing the CPU 31B and the like.

The CPU 31B outputs the result calculated by the CPU 31B and various types of data stored in the memory 30B to the P/S 37B via a signal line 31b1, a driver 38B, and a signal line 38b1. The various types of signals and data outputted from a S/P converting section 39B (abbreviated as S/P in the specification hereinafter in the drawings) is inputted to the CPU 31B via a signal line 38b2, the driver 38B and a signal line 38b2.

The reset circuit 32B performs reset according to the timing of a power source supplied from the processor 4 being changed and the timing based on the watchdog timer in the CPU 31B.

A switch ON/OFF signal generated in response to operation of each switch of the operation switching section 28B is outputted to the P/S 37B via a signal line 28b. Here, the switch ON/OFF signal generated in response to operation of each switch of the operation switching section 28B is generated with the driving voltage supplied from the driving circuit 71 of the processor 4.

The P/S 37B generates a serial signal by performing parallel/serial conversion on the switch ON/OFF signal inputted via the signal line 28b, a digital signal inputted via the signal line 36b, various types of data and calculated result inputted via the signal line 38b1 and outputs the serial signal to the processor 4 via a transceiver 40B and a signal line arranged to be inserted through the cable 33B.

The S/P 39B subjects various types of signals and data which are outputted from the processor 4 and inputted as serial signals via the signal line arranged to be inserted through the cable 33B and a receiver 41B to serial/parallel conversion and then outputs the various types of signals and data that are in the parallel form to the driver 38B via the signal line 38b2 and also to a D/A converting section (abbreviated as D/A in the specification hereinafter and the drawings) 42B via a signal line 42b.

The D/A 42B converts a CCD driving signal that is generated at the processor 4 based on the endoscope connection detecting signal among the various types of signals and data outputted from the S/P 39B into an analog signal and then outputs the analog signal to the CCD 24B via the signal line 24b2. Then, the CCD 24B is driven in accordance with a CCD driving signal inputted via the signal line 24b2.

Either or both of the endoscopes 2A and 2B may be a flexible endoscope or a rigid endoscope.

All or at least one of the P/S 37B, the S/P 39B, the driver 38B, the CPU 31B, and the memory 30B may be a FPGA for the purpose of downsizing the endoscope 2B.

As shown in FIG. 1D, the light equipment 3 has a lamp 51 for emitting a white light, an RGB filter 52 for converting the white light emitted from the lamp 51 into a light frame-sequentially of the RGB, special light filters (three for example) 53A, 53B and 53C for generating a narrow-band light by cutting a wavelength of a predetermined band of white light emitted from the lamp 51, an iris 54 for controlling an amount of the white light emitted from the lamp 51, an RGB filter 52, and a light equipment controlling unit 55 that inserts and strips special light filters 53A, 53B, and 53C into and from an outgoing optic axis for the white light emitted from the lamp 51 in accordance with a light-controlling signal to be described later.

As shown in FIG. 1D, the light equipment 3 has an operation panel 56 that enables a user to perform various settings and operational direction including adjustment of an amount of outgoing illumination, power supply ON/OFF of the device, turning ON/OFF of the lamp 51 and switching of transparent illumination and filters, a memory 57 for storing a program and various types of data, a CPU 58, a D/A converting section (abbreviated as D/A in the specification hereinafter and the drawings) 59 for converting a digital-light-controlling signal outputted from the processor 4 into an analog-light-controlling signal and outputting the analog-light-controlling signal to a filter-switching/iris-controlling unit, and a connector 60.

The light equipment 3 is detachably connected to the processor 4 via a connector 62 provided for the other end of a cable 61 extending from the connector 60.

The light equipment controlling unit 55 detects amount of light-information, which is information on the amount of the white light emitted from the lamp 51, and outputs the detected amount of light information to the processor 4 via the D/A 59 and a signal line 59a as an amount-of-light-detecting signal.

The memory 57 is a non-volatile memory, such as any of an EEPROM, a FLASH ROM, FRAM, an FeRAM, an MRAM, an OUM or an SRAM with a battery. The memory 57 stores amount of light-adjustment data, a life time of the lamp 51, the serial number of the device, an RGB filter 52, types of special light filters 53A, 53B, and 53C, maintenance information and the like as the abovementioned various types of data.

The CPU 58 includes a SIO (Serial Input/Output) 58A and a PIO (Parallel input/output) 58B. The CPU 58 controls reading and writing of various types of data to and from the memory 57 via either the SIO 58A or the PIO 58B, and also controls over the light equipment controlling unit 55 and the operation panel 56. Either a parallel interface or a serial interface may be used for writing and reading data between the CPU 58 and the memory 57. The configuration is also employed between the CPU 31B and the memory 30B and between the CPU 31A and the memory 30A.

The CPU 58 exchanges the result calculated by the CPU 58 and various types of data stored in the memory 57 with the processor 4 via a signal line 58a. The signal line 58a is connected to the CPU 58 by an end to be inserted through the cable 61 and connected to the inner circuit of the processor 4 by the other end.

The CPU 58 outputs various types of signals and data from the SIO 58A to the signal line 58a. The various types of signals and data outputted to the signal line 58a are inputted to the inner circuit of the processor 4.

The D/A 59 converts a digital-light-controlling signal outputted from the processor 4 and inputted via the signal line 59a into an analog-light-controlling signal, and outputs the analog-light-controlling signal to the filter switching/iris-controlling unit. The signal line 59a is connected to the D/A 59 by an end to be inserted through the cable 61 and connected to the inner circuit of the processor 4 by the other end. The light-controlling signal to be inputted to the D/A 59 includes information such as brightness information of an image according to the subject's image picked up by the endoscopes 2A and (or) 2B and photometry information. Data format of the light-controlling signal to be input to the D/A 59 may be any of the parallel, asynchronous serial and asynchronous format.

A ground point 63 provided in the light equipment 3 is connected to the signal line 63a. When the connector 62 is connected to the processor 4, a light source detecting signal for determining whether the model of the light equipment 3 is capable of communicating with the processor 4 or not, for example, is outputted from the ground point 63 to the processor 4 via the signal line 63a.

Each type of setting, operation directions and the like that are performed at the operation panel 56 while the light equipment 3 is connected to the processor 4 is outputted to the processor 4 via the SIO 58A of the CPU 58.

As shown in FIG. 1E, the processor 4 has a driving circuit 71, an image processing unit 72 for performing various processing on an image according to a subject's image picked up by the endoscope 2A (2B), an image compressing unit 73, an image decompressing unit 74, a main controlling unit 75 for controlling each unit of the processor 4, a front panel 76 that is capable of performing various settings and operation direction on the processor 4 and the like, an expansion controlling unit 77 that is detachably provided for the processor 4 as one or more expansion boards that can be replaced with another board having a desired function.

The driving circuit 71 generates a CCD driving signal for driving the CCD 24B based on an endoscope connection detecting signal that is outputted from the endoscope 2B and inputted via a receiver 78 and an S/P converting section (abbreviated as S/P in the specification hereinafter and the drawings) 79, and outputs the CCD driving signal to a P/S converting section (abbreviated as P/S in the specification hereinafter and the drawings) 80 via a signal line 71a. The driving circuit 71 also generates a driving signal for driving a memory 30B, a CPU 31B, and a reset circuit 32B of the endoscope 2B, and outputs the driving signal to the P/S 80 together with the CCD driving signal.

The driving circuit 71 further generates a CCD driving signal for driving the CCD 24A based on an endoscope connection detecting signal that is generated at the connector 29A and outputs the CCD driving signal to the endoscope 2A via the signal line 24a2. The driving signal for driving the memory 30A, the CPU 31A and the reset circuit 32A of the endoscope 2A may be common with the CCD driving signal or separately transmitted from a dedicated power source line.

Configuration of each of the image processing unit 72, the image compressing unit 73, the image decompressing unit 74, the main controlling unit 75, and the expansion controlling unit 77 in the processor 4 will be detailed later. The image processing unit 72, the image compressing unit 73, the image decompressing unit 74 and main controlling unit 75 in the processor 4 may be provided on a board or may be adopted to be able to replaced with another board as the expansion controlling unit 77.

Signals may be transmitted among units of the processor 4 in a parallel system or a differential serial system such as the LVDS, the RSDS or the LVPECL for reducing noise or downsizing the system. Each of the signals may be transmitted in an encrypted form among units of the processor 4. That protects the signals from being exposed to the outside of the board during the transmission among units of the processor 4 so that security of the processor 4 is improved.

The S/P 79 performs serial/parallel conversion on various types of signals and data that are outputted from the endoscope 2B and then inputted as a serial signal via a signal line that is laid to be inserted through the cable 33B and the receiver 78, and then outputs the various types of signals and data that are in the parallel form to the image processing unit 72.

The P/S 80 generates a serial signal by performing parallel/serial conversion on the signal outputted from the image processing unit 72 and then inputted via the signal line 72a and a driver 82 and the CCD driving signal outputted from the driving circuit 71 and then inputted via the signal line 71a, and outputs the serial signal to the endoscope 2B via the transceiver 81 and the signal line that is laid to be inserted through the cable 33B.

The receiver 78 and the transceiver 81 provided for the processor 4 of the present embodiment or the receiver 41B and the transceiver 40B of the endoscope 2B have insulation circuits, which are not shown.

Specifically, the image processing unit 72 of the processor 4 has the configuration as shown in FIG. 2A (as described below).

The image-pick up signals outputted via the signal line 24a1 is subjected to CDS processing by the CDS circuit 91 of the image processing unit 72, converted into a digital form by the A/D converting section (abbreviated as A/D in the specification hereinafter and the drawings) 92, converted into a predetermined frequency (for example, 13.5 MHz) by a frequency converter (not shown), and then inputted into a selector 94 through an insulation circuit 93 formed by a photo-coupler and the like.

The endoscope connection detecting signal outputted via the signal line 29a, various types of signals and data outputted via the signal line 31a, and a switch ON/OFF signal outputted via the signal line 28a are inputted into the selector 94 through the insulation circuit 93.

Further, into the selector 94, the image pickup signal and the endoscope connection detecting signal, which are output signals from the S/P 79, are inputted via the signal line 79b, the switch ON/OFF signal is inputted via the signal line 79c, and various types of signals and data are inputted via the driver 82 and the signal line 82a.

The selector 94 detects the connection status of the endoscope 2A and the endoscope 2B based on the endoscope connection detecting signal that is inputted via the signal line 29a and the endoscope connection detecting signal that is inputted via the signal line 79b among inputted signals. In any one of the cases where the selector 94 detects that none of the endoscope 2A and the endoscope 2B are connected to the processor 4, where both the endoscope 2A and the endoscope 2B are connected to the processor 4, and where only the endoscope 2B is connected to the processor, the selector 94 makes the image pickup signal that is inputted via the signal line 79b outputted to the signal line 94a, makes the endoscope connection detecting signal that is inputted via the signal line 79b and the switch ON/OFF signal that is inputted via a signal line 79c outputted to the signal line 94b, and makes various types of signals and data that are inputted via the signal line 82a inputted and outputted to and from to a signal line 94c. In the case where only the endoscope 2A is connected to the processor, the selector 94 makes the image pickup signal that is inputted via an insulation circuit 93 outputted to the signal line 94a, makes the endoscope connection detecting signal and switch ON/OFF signal that are inputted via the insulation circuit 93 to the signal line 94b, and makes various types of signals and data that are inputted via the insulation circuit 93 inputted and outputted to and from the signal line 94c.

In the case where both the endoscope 2A and the endoscope 2B are connected to the processor 4, the selector 94 may make the image pickup signal that is inputted via the insulation circuit 93 outputted to the signal line 94a, make the endoscope connection detecting signal and the switch ON/OFF signal that are inputted via the insulation circuit 93 outputted to the signal line 94b, and make the various types of signals and data that are inputted via the insulation circuit 93 outputted to the signal line 94c, or may make the signal obtained by the previously connected endoscope outputted so that the processing for displaying the image (on a display unit such as a monitor) is performed. In the case where both of the endoscope 2A and the endoscope 2B are connected to the processor 4, a graphic circuit 106H (or 106S) (to be described later) among units arranged at the post-stage of the selector 94 in the processor 4 may generate and output an alert display image directing a parallel connection as shown in FIG. 2B. In the case where the selector 94 detects that one of the endoscopes is detached, the selector 94 may be adopted to automatically output an image obtained by the other selector.

As a result, in the case where both of the endoscopes 2A and 2B are connected, the processor 4 is capable of asking a user to remove one of the endoscopes as soon as possible. Also as a result, the processor 4 automatically displays an image of the other endoscope that is connected when one of the endoscopes is removed. Accordingly, the user can perform examination easily and smoothly. That improves efficiency of the examination, and reduces a time period for examination.

In the case where both of the endoscope 2A and the endoscope 2B are connected to the processor 4, each unit arranged at the post-stage of the selector 94 in the processor 4 may cause an LED (not shown) provided on the front panel 76 and (or) the keyboard 5 to be lit or flickered for alerting. In the case where both of the endoscope 2A and the endoscope 2B are connected to the processor 4, each unit arranged at the post-stage of the selector 94 in the processor 4 may cause a beeper (not shown) to sound.

The image pickup signal outputted from the selector 94 to the signal line 94a is subjected to the OB (Optical Black) clamp processing, frequency conversion (for example 27 MHz), white balancing and AGC (Automatic Gain Control) by the pre-stage image processing circuit 95, and then outputted to a freeze circuit 96 as an image signal. The endoscope connection detecting signal and the switch ON/OFF signal that are outputted from the selector 94 to the signal line 94b are outputted to the main controlling unit 75 (to PIO143 to be described later of the main controlling unit 75) (denoted by A1 in the figure). The various types of signals and data that are outputted from the selector 94 to the signal line 94c are inputted and outputted to and from the main controlling unit 75 (to SIO142 to be described later of the main controlling unit 75) (denoted by A2 in the figure).

An image signal outputted from a pre-stage image processing circuit 95 is inputted to a freeze circuit 96. When a first freeze switch (hereinafter referred to as freeze switch) is operated and first freeze direction (hereinafter referred to as freeze direction) is issued or a second freeze switch (hereinafter referred to as S freeze switch) is operated and second freeze direction (hereinafter referred to as S freeze direction) is issued in any one of operating devices, the freeze circuit 96 outputs a freeze image to the memory 97. The first freeze image obtained when the freeze direction is issued is referred to as a freeze image and the second freeze image obtained when the S freeze direction is issued is referred to as an S freeze image below. The freeze switch and the S freeze switch provided for the operating device can perform toggle operation (alternates operations of freeze ON→OFF→ON . . . each time the switches are pressed). In the present embodiment, the operating device directs the keyboard 5, the foot switch 6, the front panel 76, the operation switching sections 28A and 28B and each of HIDs (Human interface Devices) to be described later. The freeze circuit 96 may output a pre-freeze image in addition to the above-described freeze image and the S freeze image.

The image signal outputted from the freeze circuit 96 is inputted to a post-stage image processing circuit 98. The image signal inputted to the post-stage image processing circuit 98 is subjected to IHb color highlighting, moving image color shift correction, color tone adjustment in R (red) or B (blue) and γ correction and the like and outputted.

The image signal outputted from the post-stage image processing circuit 98 is outputted to each of a processing system for producing an image in SDTV (Standard Definition Television) system, which is a standard image, and a processing system for producing an image in the HDTV (High Definition Television) system, which is a high quality image. That enables the processor 4 to output an image in both output systems; the SDTV output (in case of NTSC, output corresponding to 720×480, in case of PAL, output corresponding to 720×576), and the HDTV output (output corresponding to 1920×1080).

Now, a processing system for producing an image in the SDTV system in the processor 4 will be described.

The image signal outputted from the post-stage image processing circuit 98 is subjected to zoom-up/down (processing such as electronic zoom-up/down, image resize processing and the like), edge highlighting, structure highlighting and the like by a zoom-up/highlight circuit 99S according to an operation, setting and the like in each operating device, subjected to vertical and horizontal reverse and 90-degree turning by an image turning circuit 100S, and then subjected to synchronization by a synchronization circuit 101S. In the present embodiment, the synchronization circuit 101S performs at 27 MHz when an image signal is inputted and at 13.5 NHz when an image signal is outputted.

A memory 102S is made of a non-volatile memory such as a FLASH ROM, an FRAM, an FeRAM, an MRAM, or an OUM. The memory 102S stores processing parameters including a zoom-up (down) factor, a highlighting factor and image turning parameter as parameters related to processing of a zoom-up/highlight circuit 99S and an image turning circuit 100S. A controller 103S controls processing of the zoom-up/highlight circuit 99S and the image turning circuit 100S according to each processing parameter stored in the memory 102S.

The memory 102S may be formed as a volatile memory such as an SRAM, an SDRAM, an EDORAM, a DRAM or an RDRAM, and may be adopted to allow a necessary parameter to be written in the main controlling unit 75 each time when the main power source of the processor 4 is turned on. In the description below, it is assumed that all the memories in the image processing unit 72 may employ almost the same configuration as that of the memory 102S.

A memory 104S stores each frame image of R, G (green) and B so that the synchronization circuit 101S synchronizes all the frame images to make the frame images outputted at the same time.

A thumbnail image generating circuit 105S generates a thumbnail image (also referred to as an index image) based on an image signal outputted from the synchronization circuit 101S, and stores the thumbnail image in the memory (not shown). The thumbnail image generating circuit 105S outputs the thumbnail image stored in the memory (not shown) each time when a record direction such as release or capture to a printer is issued in each operating device.

A graphic circuit 106S generates and outputs character and graphic information directing information related to an image according to the image signal that is outputted in a state synchronized by the synchronization circuit 101S (hereinafter referred to as an endoscope related information). It is assumed that the graphic information is information related to each image such as an error display, a menu display, a HELP image, a GUI, a CUI and the like.

A memory 107S is used when the graphic circuit 106S generates character and graphic information directing the endoscope related information.

A composition/masking processing circuit 108S performs masking processing on an image signal outputted in a state synchronized by the synchronization circuit 101S, and also combines the image signal with the thumbnail image generated by the thumbnail image generating circuit 105S, character and graphic information generated by the graphic circuit 106S, a synchronous circuit 122S to be described later, and an output from each of the image decompressing unit 74 and the expansion controlling unit 77, and outputs the combined image signal as an endoscope composite image. The mask data used in the masking processing may be one generated by the graphic circuit 106S or one generated by the composition/masking processing 108S itself.

A memory 109S stores the endoscope composite image generated by the composition/masking processing circuit 108S (detailed later).

The endoscope composite image outputted from the composition/masking processing circuit 108S is subjected to analog conversion at a D/A converting section (abbreviated as D/A in the specification hereinafter and the drawings) 110S and level adjustment at an adjusting circuit 111S and then outputted via a signal line 111Sa.

A processing system for generating an image in the HDTV system in the processor 4 will be described.

The image signal outputted from the post-stage image processing circuit 98 is subjected to frequency conversion (for example 74 MHz) by the frequency converting section (not shown), subjected to zoom-up/down, edge highlighting, and structure highlighting by a zoom-up/highlight circuit 99H according to the operation and setting by each operating device, subjected to the vertical and horizontal reverse and 90-degree turning by an image turning circuit 100H, and then subjected to synchronization by a synchronization circuit 101H.

A memory 102H stores processing parameters including a zoom-up (down) factor, a highlighting factor and image turning parameter as parameters related to processing of the zoom-up/highlight circuit 99H and the image turning circuit 100H. A controller 103H controls processing of the zoom-up/highlight circuit 99H and the image turning circuit 100H according to each processing parameter stored in the memory 102H.

A memory 104H stores each frame image of R, G (green) and B so that the synchronization circuit 101H synchronizes all the frame images to make the frame images outputted at the same time.

A thumbnail image generating circuit 105H generates a thumbnail image based on an image signal outputted from the synchronization circuit 101H, and stores the thumbnail image in the memory (not shown). The thumbnail image generating circuit 105H outputs the thumbnail image stored in the memory (not shown) each time when a record direction such as release or capture to a printer is issued in each operating device. Each processing performed when a thumbnail image is generated at the thumbnail image generating circuits 105H and 105S may be those performed by the thumbnail image generating circuit 224 in FIG. 5 to be described later. The thumbnail image generated at the thumbnail image generating circuit 224 may be outputted via the synchronous circuit 252 shown in FIG. 6 to be described later and the like and outputted (and displayed) in a state combined by the composition/masking processing circuit 108H or 108S.

A graphic circuit 106H generates and outputs characters and graphic information directing information related to an image according to the image signal that is outputted in a state synchronized by the synchronization circuit 101H (hereinafter referred to as an endoscope related information). It is assumed that the graphic information is information related to each image such as an error display, a menu display, a HELP image, a GUI, a CUI and the like.

A memory 107H is used when the graphic circuit 106H generates character and graphic information directing the endoscope related information.

A composition/masking processing circuit 108H performs masking processing on an image signal outputted in a state synchronized by the synchronization circuit 101H, and also combines the image signal with the thumbnail image generated by the thumbnail image generating circuit 105H, character and graphic information generated by the graphic circuit 106H, a synchronous circuit 122H to be described later, and an output from each of the image decompressing unit 74 and the expansion controlling unit 77, and outputs the combined image signal as an endoscope composite image. The mask data used in the masking processing may be one generated by the graphic circuit 106H or one generated by the composition/masking processing 108H itself.

A memory 109H stores images including the endoscope composite image generated by the composition/masking processing circuit 108H.

When a freeze direction or a S freeze direction is issued in any one of the operating devices, a memory 112H stores images such as a freeze image and (or) an S freeze image that is outputted from the composition/masking processing circuit 108H. The processing related to each image inputted and outputted at the memory 112H will be detailed in the description for FIG. 20A and FIG. 20B.

The endoscope composite image outputted from the composition/masking processing circuit 108H is subjected to analog conversion at the D/A converting section (abbreviated as D/A in the specification hereinafter and the drawings) 110H and level adjustment at an adjusting circuit 111H and then outputted via a signal line 111Ha.

The image I/O processing section 121 encodes either the endoscope composite image outputted from the composition/masking processing circuit 108S or the endoscope composite image outputted from the composition/masking processing circuit 108H so that the image (as a digital image or an analog image) can be outputted via an interface such as the LVDS, the SDI, the H-SDI, the DV (IEEE1394), the DVI, the D1, the D2, the D3, the D4, the D5, the D6, the D9, or the HDMI, and then outputs each of the endoscope composite images via a signal line 121a.

The image I/O processing section 121 performs decoding (including digitizing processing by A/D conversion) on the image inputted via the signal line 121a and the interface and outputs the image to the synchronous circuits 122S and 122H as an RGB signal (or YCrCb signal).

The synchronous circuit 122S performs SDTV synchronization based on a synchronizing signal outputted from a synchronizing signal generating circuit (abbreviated as SSG hereinafter) 123 (to be described later) on the RGB signals outputted from the image I/O processing section 121 so that the RGB signals are composed by the composition/masking processing circuit 108S at more appropriate timing, and then outputs the RGB signal processed with the SDTV synchronization to the composition/masking processing circuit 108S (denoted by A4 in the figure).

The synchronous circuit 122H performs HDTV synchronization based on a synchronizing signal outputted from SSG123 on the RGB signals outputted from the image I/O processing section 121 so that the RGB signals are composed by the composition/masking processing circuit 108H at more appropriate timing, and then outputs the RGB signal processed with the HDTV synchronization to the composition/masking processing circuit 108H (denoted by A3 in the figure).

The selector 124 selects one of the endoscope composite image that is outputted from the composition/masking processing circuit 108S (a moving image) and the endoscope composite image that is outputted from the composition/masking processing circuit 108H (a moving image) and outputs the selected endoscope composite image via a signal line 124a.

The controller/selector 125 generates an image to be outputted according to the type of the peripheral device connected to the processor 4 and stores the image to be outputted in memory 126 based on an endoscope composite image (still image) outputted from the composition/masking processing circuit 108S and an endoscope composite image (still image) outputted from the composition/masking processing circuit 108H each time when a record direction such as release or capture to a printer is issued in each operating device. The controller/selector 125 synchronizes the image to be outputted stored in the memory 126 so that the image to be outputted is to be processed by the image compressing unit 73 at more appropriate timing, and then outputs the image to be outputted processed by the synchronization via the signal line 125a. The memory 126 may be made of a ring buffer.

Now, an inner configuration of the controller/selector 125 will be described.

As shown in FIG. 2F, the controller/selector 125 includes a memory controller 125A, memories 125B and 125C, and a selector 125D. A clock signal of 100 MHz that is generated in the SSG 123, for example, is inputted in each unit of the controller/selector 125.

The memory controller 125A controls I/O of the memories 125B and 125C and the memory 126 based on such a signal as a clock signal outputted from the SSG 123 and control by the main controlling unit 75.

The memory 125B is formed as a FIFO memory (or a line memory). The memory 125B can serially store and output the image to be outputted that is outputted from the composition/masking processing circuit 108H by one frame (or by one line) based on a clock signal of 74 MHz that is generated by the SSG 123. The memory 125C is formed as a FIFO memory (or a line memory). The memory 125C can serially store and output the image to be outputted that is outputted from the composition/masking processing circuit 108S by one frame (or by one line) based on a clock signal of 13.5 MHz that is generated by the SSG 123.

The selector 125D selectively outputs an output from either the memory 125B or the memory 125C to either the signal line 125a or the memory 126.

The clock signal of 100 MHz that is to be inputted to each unit of the controller/selector 125 and the image compressing unit 73 is replaced with the clock signal of 74 MHz, a moving image can be outputted to the image compressing unit 73. The abovementioned 74 MHz is correctly described as either (74.25/1.001) MHz or 74.25 MHz. That is the same in the description below. In such a case, the image compressing unit 73 may be made as a programmable circuit such as an FPGA, a DSP, or a dynamic reconfigurable processor, and may also be adopted to switch the functions to work as a circuit with a function of compressing a still image or a circuit with a function of compressing a moving image. (The image compressing unit 73 used in the processor 4 of the present embodiment will be detailed in the description with reference to FIG. 5.)

When the image compressing unit 73 is made as a programmable circuit, the image compressing unit 73 may be adopted to select the compressing form (any one of JPEG, JPEG2000, TIFF, BMP, AVI, MPEG, H.264 OR WMV) on the setting screen and the like shown in FIG. 8 to be described later and download a block (firmware or configuration data) according to the selection. The block can be downloaded by a CPU 151 of the expansion controlling unit 77A via a bus bridge 163 or from a ROM or the like (not shown) provided for the image compressing unit 73. An error message directing that download is being performed can be displayed on the endoscope composite image while the block is being downloaded, or a predetermined LED (not shown) of the operating device can be lit (or flickered). When the block has been downloaded, a message directing the completion of the download can be displayed on the screen.

The SSG 123 provided for the processor 4 outputs two or more vertical synchronizing signals and horizontal synchronizing signals, an ODD/EVEN determining signal and a clock as a signal according to the type of the endoscope 2A and the endoscope 2B based on the endoscope connection detecting signal outputted from the endoscope 2A via the signal line 29a and the insulation circuit 93 or the endoscope connection detecting signal outputted from the endoscope 2B via the signal line 79b.

Among the signals outputted from the SSG 123, a vertical synchronizing signal VD1 (for example, 60 Hz) and a horizontal synchronizing signal HD1 (for example, 15.75 kHz) are outputted to each unit from the CDS circuit 91 to the post-stage image processing circuit 98, each unit from the zoom-up/highlight circuit 99S to the memory 104S, and each unit from the zoom-up/highlight circuit 99H to the memory 104H. Among the signals outputted from the SSG 123, a vertical synchronizing signal VD2 (for example, 50 Hz or 60 Hz), a horizontal synchronizing signal VD3 (for example, 50 Hz or 60 Hz), an ODD/EVEN determining signal ODD2, an ODD/EVEN determining signal ODD3, a horizontal synchronizing signal HD2 (for example, 15.75 kHz or 15.625 kHz) and a horizontal synchronizing signal HD3 (for example, 33.75 kHz or 28.125 kHz) are outputted to the synchronization circuit 101S, each unit from the memory 104S to the memory 109S, the synchronous circuit 122S, the synchronization circuit 101H, each unit from the memory 104H to the memory 109H, the memory 112H, the synchronous circuit 122H, the image I/O processing section 121, the selector 124, the controller/selector 125, and the memory 126.

The SSG 123 outputs a clock signal of 13.5 MHz, which is a standard clock in the SDTV system, a clock of 27 MHz with a frequency double of that of the standard clock, and a clock signal of 74 MHz, which is a standard clock in the HDTV system respectively, as a clock signal mainly used in image processing.

Among the clock signals, the clock signal of 13.5 MHz, for example, is outputted to each unit from the A/D 92 to the pre-stage image processing circuit 95, each unit from the zoom-up/highlight circuit 99S to the memory 104S, the D/A 110S, the image I/O processing section 121, the synchronous circuit 122S, the selector 124, and the controller/selector 125. Among the clock signals, the clock signal of 27 MHz, for example, is outputted to each unit from the pre-stage image processing circuit 95 to the post-stage image processing circuit 98, each unit from the zoom-up/highlight circuit 99S to the controller 103S, and the image I/O processing section 121. Among the clock signals, the clock signal of 74 MHz, for example, is outputted to the synchronous circuit 122H, each unit from zoom-up/highlight circuit 99H to the D/A 110H, the memory 112, the image I/O processing section 121, the synchronous circuit 122H, the selector 124, and the controller/selector 125.

Specifically, the main controlling unit 75 of the processor 4 has a configuration shown in FIG. 2C, for example.

The CPU 131 of the main controlling unit 75 controls writing and reading of data in RAMs 132 and 133 via a parallel interface (or a serial interface) (not shown) and a system bus 131a.

The RAMs 132 and 133 are adopted as a volatile memory such as the SRAM, the SDRAM, the DRAM and the RDRAM. The RAMs can store program related data, endoscope information data, endoscope image data and the like and can also be used as a cache.

The CPU 131 of the main controlling unit 75 controls a real-time clock (abbreviated as RTC in the specification hereinafter and the drawings) 134 that is formed by a clock or the like and responsible for time management via the system bus 131a.

The CPU 131 of the main controlling unit 75 controls ROMs 135 and 136 that store each type of data such as program data, data on program version and the like via the system bus 131a.

The CPU 131 of the main controlling unit 75 controls backup for a RAM 137 via the system bus 131a.

The backup RAM 137 is made of an EEPROM, a FLASH ROM, an FRAM, an FeRAM, an MR AM, an OUM, an SRAM with battery and the like. The backup RAM 137 stores endoscope related information as information that should be kept after the processor 4 is turned off including a log of program operations, maintenance information, setting information of the front panel 69 and the keyboard 14, various types of setting screen information, white balance data and the like.

The CPU 131 of the main controlling unit 75 controls an address decoder 138 that outputs a chip select signal to each unit of the processor 4 and a bus driver (abbreviated as BUF in the specification hereinafter and the drawings) 139 for providing signals of the system bus 131a to each unit of the processor 4 via the system bus 131a.

The CPU 131 of the main controlling unit 75 controls a RESET circuit 140 and also controls a timer 141 that is responsible for time management via the system bus 131a.

The RESET circuit 140 has a watchdog timer and the like (not shown) and performs reset when it is detected either that the processor 4 is switched on or that a program running in the processor 4 is hanged up.

The CPU 131 of the main controlling unit 75 controls the SIO 142 and the PIO 143 via the system bus 131a.

The SIO 142 can communicate with the SIO 58A of each unit of the processor 4 (e.g., the SIO of the expansion controlling unit 77, each unit of the front panel 76 and the image processing unit 72), a peripheral device connected to the processor 4, the keyboard 5, the CPU 31A of the endoscope 2A, the CPU 31B of the endoscope 2B, the CPU 58 of the light equipment 3 and the like via a serial interface. The serial interface may be any of a start-stop system, a clock system, a USB (Registered Trademark) HOST/DEVICE, CAN, FLEX RAY, 12C and the like. Connection between the SIO 142 and the SIO of the expansion controlling unit 77 is denoted by B1 in the figure. The signal line for connecting the SIO 142 and a peripheral device is denoted by 142a in the figure.

The PIO 143 can communicate with each unit of the processor 4 (e.g., the PIO and a board connection information storing circuit of the expansion controlling unit 77, and each unit of the image processing unit 72), a peripheral device connected to the processor 4, an operation switch 28 of the endoscope 2A, the operation switch 28 of the endoscope 2B, the foot switch 6 and the like via a parallel interface. Connection between the PIO 143 and the PIO of the expansion controlling unit 77 is denoted by B2 in the figure. The signal line for connecting the PIO 143 and a peripheral device is denoted by 143a in the figure.

The PIO 143 outputs an endoscope connection detecting signal that is inputted via the signal line 94b, a light equipment detecting signal that is inputted via the signal line 63a to the CPU 131 via the system bus 131a. The PIO 143 outputs the light-controlling signal that is generated and outputted by the CPU 131 to the light equipment controlling unit 55 via the signal line 59a and the D/A 59. The PIO 143 outputs a board connection detecting signal outputted from the expansion controlling unit 77 to the CPU 131 via the system bus 131a. Connection of the route through which the board connection detecting signal is transmitted from the expansion controlling unit 77 to the PIO 143 is denoted by B3 in the figure.

The CPU 131 of the main controlling unit 75 controls a controller 144 and the memory 145 via the system bus 131a.

The controller 144 communicates with a peripheral device that is connected via the signal line 144a by using a token passing protocol such as the Token Ring, the FDDI, the Circlink or the Arcnet and the like.

The memory 145 stores shared information, log information and the like with a peripheral device that is connected via the signal line 144a.

In the present embodiment, each part of the main controlling unit 75 including the CPU 131, the RAM 132, the ROM 135, the address decoder 138, the reset circuit 140, the timer 141, the SIO 142, the PIO 143, the controller 144 and the memory 145 is made of a dedicated IC, though, the configuration is not limited to that and each unit may be made of a programmable IC such as the FPGA, the DSP or a reconfigurable processor. Each part of the image processing unit 72, the image compressing unit 73, the image decompressing unit 74 and the expansion controlling unit 77 with the same function as that of each part of the main controlling unit 75 is not limited to a dedicated IC and may be made of a programmable IC.

When the CPU 131 of the main controlling unit 75 detects that the signal level of the light source detecting signal that is inputted via the PIO 143 is at the L level, for example, based on the light source detecting signal, the CPU 131 determines that communication is available with the light equipment 3 (that the light equipment 3 has a communication function). When the CPU 131 of the main controlling unit 75 detects that the signal level of the light source detecting signal that is inputted via the PIO 143 is at the H level, for example, based on the light source detecting signal, the CPU 131 determines that communication is unavailable with the light equipment 3 (that the light equipment 3 has no communication function).

Each operation performed by selector 94 based on the endoscope connection detecting signal may be performed by the CPU 131 of the main controlling unit 75 based on table data stored in the ROM 135 when the endoscope connection detecting signal is inputted via the signal line 29a or the signal line 79b.

The expansion controlling unit 77, which is configured as an expansion board to be detachably connected to the processor 4, is specifically configured as the expansion controlling unit 77A with network communicating functions as shown in FIG. 2D (and as below).

The CPU 151 of the expansion controlling unit 77A controls reading and writing of data in the RAM 152 via a parallel interface (or a serial interface) (not shown) and the system bus 151a.

The RAM 152 is adopted as a volatile memory such as the SRAM, the SDRAM, the DRAM or the RDRAM. The RAM can store program related data, endoscope information data, endoscope image data and the like and can also be used as a cache.

The CPU 151 of the expansion controlling unit 77A controls a real-time clock (abbreviated as RTC in the specification hereinafter and the drawings) 153 that is formed by a clock or the like and responsible for time management via the system bus 151a.

The CPU 151 of the expansion controlling unit 77A controls ROM 154, which stores data such as the program data, the data on the program version, the MAC address, IP address and the like of the Ethernet (Registered Trademark), via the system bus 151a.

The CPU 151 of the expansion controlling unit 77A controls a backup RAM 155 via the system bus 151a.

The ROM 154 and the backup RAM 155 are formed by an EEPROM, a FLASH ROM, an FRAM, a FeRAM, an MRAM, an OUM, an SRAM with a battery and the like. The backup RAM 155 stores endoscope related information as information that should be kept after the processor 4 is turned off including a log of program operations, maintenance information, setting information of the front panel 69 and the keyboard 14, various types of setting screen information, white balance data and the like.

The CPU 151 of the expansion controlling unit 77A controls an address decoder 156 that outputs a chip select signal to each unit of the processor 4 via the system bus 151a.

The CPU 151 of the expansion controlling unit 77A controls a RESET circuit 157 and also controls a timer 158 that is responsible for time management via the system bus 151a.

The RESET circuit 157 has a watchdog timer and the like (not shown) and performs reset when it is detected either that the processor 4 is switched on or that a program running in the processor 4 is hanged up.

The CPU 151 of the expansion controlling unit 77A controls the SIO 159 and the PIO 160 via the system bus 151a.

The SIO 159 can communicate with each unit of the processor 4 (e.g., the image I/O processing section 121, SIO of the controller/selector 125 and the main controlling unit 75), and a peripheral device connected to the processor 4 via a serial interface. The serial interface may be any of a start-stop system, a clock system, a USB (Registered Trademark) HOST/DEVICE, CAN, FLEX RAY, 12C and the like.

The PIO 160 can communicate with each unit of the processor 4 (e.g., the PIO of the image compressing unit 73, the image decompressing unit 74, the image I/O processing section 121, the controller/selector 125 and the main controlling unit 75), and a peripheral device connected to the processor 4 via a parallel interface.

The CPU 151 of the expansion controlling unit 77A controls a controller 161 and a HUB 162 via the system bus 151a.

The controller 161 includes a circuit and middleware in the MAC layer and the physical layer of the Ethernet (Registered Trademark) to be able to communicate by using the Ethernet (Registered Trademark). Thus, the controller 161 can communicate with a peripheral device connected to the processor 4 via the HUB 162 and the signal line 162a that is connected to the HUB 162.

The CPU 151 of the expansion controlling unit 77A controls the bus bridge 163 via a system bus 151b. The system bus 151b may be any of a PCI, the RA PIDIO, the PCI-X, the PCI EXPRESS, the COMPACT PCI, ISA and the like. Connection between the bus bridge 163 and the image compressing unit 73 is denoted by C1 and C2 in the figure. Connection between the bus bridge 163 and the image decompressing unit 74 is denoted by C3 and C4 in the figure.

The CPU 151 of the expansion controlling unit 77A controls the controller 164 as a USB (Registered Trademark) interface via the system bus 151b and the bus bridge 163.

The CPU 151 of the expansion controlling unit 77A controls a card controller 165 via the system bus 151b and the bus bridge 163.

The card controller 165 controls a PC card 167 and a memory card 168 which serves as image recording unit connected to a slot (not shown). The memory card 168 may be any of a COMPACT FLASH (Registered Trademark), the SMART MEDIA (Registered Trademark), an SD card, a miniSD (Registered Trademark) card, a memory card in a PC card form, a flash drive, an HDD, a multi media card, an xDPicture card and a Memory Stick (Registered Trademark).

The card controller 165 controls a buffer 166. Even if the processor 4 is switched off before data has been transmitted or received, for example, during communication between the controller 161 and a peripheral device, the buffer 166 which serves as an image recording unit can store the data which has not been transmitted or received to prevent the data from being deleted. The buffer 166 may be any of the COMPACT FLASH (Registered Trademark), the SMART MEDIA (Registered Trademark), an SD card, the miniSD (Registered Trademark) card, a memory card in the PC card form, a flash drive, an HDD, a multi media card, an xDPicture card, the Memory Stick (Registered Trademark) or a PC card. The USB (Registered Trademark) (not shown) memory connected to the controller 164 may be used in the place of the buffer 166.

The CPU 131 of the main controlling unit 75 and the CPU 151 of the expansion controlling unit 77A can determine whether the buffer 166 is on the way of recording or not by storing the information being recorded in the backup RAM 137 of the main controlling unit 75 or the backup RAM 155 of the expansion controlling unit 77A.

The CPU 151 of the expansion controlling unit 77A controls the graphic circuit 169 via the system bus 151b and the bus bridge 163.

The graphic circuit 169 performs graphic processing related to a moving image, a still image, WEB display and the like based on the synchronizing signal outputted from the SSG 123 of the image processing unit 72. Connection between the graphic circuit 169 and the composition/masking processing circuit 108H and the composition/masking processing circuit 108S of the image processing unit 72 is denoted by A5 and A6 in the figure.

The CPU 151 of the expansion controlling unit 77A controls encrypting circuit 170 via the system bus 151b and the bus bridge 163.

The encrypting circuit 170 is adopted as a circuit capable of adding and detecting security information and performing encryption and decryption for communicating with the peripheral device. The encrypting circuit 170 may use any of the 3DES SSL RSA and elliptic curve cryptosystem in encryption and can support either the Ipsec or the SSL protocol.

The expansion controlling unit 77A has a board connection information storing circuit 171 that outputs the board connection detecting signal to the PIO of the main controlling unit 75 when the expansion controlling unit 77A is connected.

The board connection detecting signal outputted from the board connection information storing circuit 171 may be a pull-down signal to two or more GND or a pull-up signal to a power source. The board connection information storing circuit 171 may be adopted as a nonvolatile memory that stores information on the type of the expansion controlling unit 77A. The board connection information storing circuit 171 may output the board connection detecting signal to the SIO of the main controlling unit 75 via a serial interface (not shown).

When the expansion controlling unit 77A has a radio controlling circuit that can be connected in any of the bus bridge 163, the controller 164, or a slot for the PC card 167 and the memory card 168 to be inserted, for example, the expansion controlling unit 77A can wirelessly communicate with a peripheral device connected to the processor 4. As an antenna, a memory, and an encrypting circuit according to the radio controlling circuit are installed to each unit of the endoscope 2A, the endoscope 2B and a treatment instrument for an endoscope (not shown), endoscope related information can be exchanged with each unit wirelessly.

The expansion controlling unit 77 that is one or more expansion boards detachably connected to the processor 4 is not limited to be connected with only the expansion controlling unit 77A and may also be connected with the expansion controlling unit 77B having a zoom-controlling function and some functions of the endoscope form detecting device as shown in FIG. 2E (and described below).

The CPU 181 of the expansion controlling unit 77B controls the RAM 152, the ROM 154, the address decoder 156, the reset circuit 157, the timer 158, the SIO 159 and the PIO 160, which are units with the same configurations as those mentioned above, via the system bus 181a. The CPU 181 of the expansion controlling unit 77B controls the graphic circuit 169 with the same configuration as that mentioned above via the system bus 181b.

The expansion controlling unit 77B has a board connection information storing circuit 182 that outputs the board connection detecting signal to the PIO of the main controlling unit 75 (different from the board connection information storing circuit 171) when the expansion controlling unit 77B is connected.

Now, a configuration and functions of an endoscope form detecting device 1001 shown in FIG. 2E will be described.

The endoscope form detecting device 1001 includes a source coil driving circuit 1001A, a sense coil 1001B, a sense coil signal amplifying circuit 1001C, and an A/D converter (abbreviated as ADC in the specification hereinafter and the drawings) 1001D.

The source coil driving circuit 1001A generates a magnetic field in two or more source coils 25A of the endoscope 2A and two or more source coils 25B of the endoscope 2B by outputting driving signal currents in sine waves with different frequencies to the source coils of the endoscope 2A and the endoscope 2B. Frequencies of the driving signal currents are set based on driving frequency setting data (also referred to as driving frequency data) stored in driving frequency setting data storing means or driving frequency setting data memory means (not shown) of the source coil driving circuit 1001A. Connection between the source coil driving circuit 1001A and the endoscope 2A and the endoscope 2B is denoted by D1 in the figure.

Magnetic fields generated by source coils 25A of the endoscope 2A and the source coils 25B of the endoscope 2B are received by the sense coil 1001B, amplified by the sense coil signal amplifying circuit 1001C, and then converted into digital data by the ADC 1001D.

The digital data generated by the ADC 1001D is outputted from the ADC 1001D under the control performed by the control signal generating section 183 of the expansion controlling unit 77B, and then inputted to a memory 185 via a receiving circuit 184. The digital data inputted in the memory 185 is read in under the control of the CPU 181.

The CPU 181 separates and extracts magnetic field detecting information on frequency components corresponding to driving frequencies of the source coils 25A and source coils 25B by performing frequency extraction (Fourier Transform: FFT) on the digital data read from the memory 185. In such a manner, the CPU 181 calculates spatial position coordinates of the source coils 25A and the source coils 25B and estimates inserting status of an insertion portion 21A of the endoscope 2A and an insertion portion 21B of the endoscope 2B based on the special position coordinates. Based on estimation by the CPU 181, display data for forming an endoscope form image is generated by a graphic circuit, and the display data is masked by the composition/masking processing circuit 108H and the composition/masking processing circuit 108S, outputted and displayed (on a display unit such as a monitor).

Now, the zoom-controlling function of the expansion controlling unit 77B will be described.

The driving circuit 186 is controlled by the CPU 131 via the SIO 142 and the PIO 143 of the main controlling unit 75. The driving circuit 186 drives the actuators 23A and 23B under the control. As a result, the objective optical systems 22A and 22B are moved in the axial directions of the insertion portion 21A and the insertion portion 21B according to respective modes of zoom-up (tele) and wide angle (wide), for example. Connection between the driving circuit 186 and the endoscopes 2A and 2B is denoted by D2 in the figure.

The CPU 131 of the main controlling unit 75 controls the graphic circuits 106S and 106H and obtains zoom control information, which is information on a zoom status (zoom-up or wide angle) when the endoscope 2A and the endoscope 2B pick up subjects' images from the driving circuit 186 of the expansion controlling unit 77B. The zoom control information obtained by the CPU 131 is made into an image by the graphic circuits 106S and 106H, masked by the composition/masking processing circuit 108H and the composition/masking processing circuit 108S, and then outputted and displayed (on a display unit such as a monitor).

Configuration for realizing the zoom-controlling function of the expansion controlling unit 77B and configuration for realizing some functions of the endoscope form detecting device are not limited to those integrated to an expansion controlling unit as mentioned above and may be those provided for different expansion controlling units and each of the expansion controlling unit may output different board connection detecting signals.

The expansion controlling unit 77 has a configuration with one or more expansion boards as mentioned above. That enables the processor 4 to easily realize two or more functions and easily set various types of functions at low costs.

Based on the board connection detecting signal outputted from the board connection information storing circuit 171 and the board connection information storing circuit 182, the CPU 131 of the main controlling unit 75 determines that only the expansion controlling unit 77A is connected if the obtained binary data is ‘000’. Then, the CPU 131 automatically causes to display (an image based on) network related information in a predetermined image size, which is outputted from the graphic circuit 169 of the expansion controlling unit 77A via connections denoted by A5 and A6 in the figure, at a predetermined position (any of the upper left, lower left, upper right and lower right on the screen) set on the setting screen shown in FIG. 8 to be described later. (The image based on) the network related information is shown like PinP image 504, which is to be described in the description of FIG. 19D, for example.

Based on the board connection detecting signal outputted from the board connection information storing circuit 171 and the board connection information storing circuit 182, the CPU 131 of the main controlling unit 75 determines that only the expansion controlling unit 77B is connected if the obtained binary data is ‘001’. Then, the CPU 131 automatically causes to display the endoscope form detected image and the zoom control information that is made into an image at the graphic circuits 106S and 106H at a predetermined position (any of the upper left, lower left, upper right and lower right on the screen) set on the setting screen shown in FIG. 8 to be described later. The endoscope form detected image is outputted from the graphic circuit 169 of the expansion controlling unit 77B via connections denoted by A5 and A6 in the figure. The endoscope form detected image and the zoom control information may be outputted in a state adjusted by the CPU 131 in positions and image sizes so that the image and information are not overlapped by each other, or may be outputted with priority for overlapping one being output (for example, in a state in which the zoom control information is displayed at the front).

The endoscope form detected image is shown like an endoscope form detected image 502 which is to be described with reference to FIG. 19D and the like. The zoom control information is shown as zoom control information 503 which is to be described later with reference to FIG. 19D and the like.

Based on the board connection detecting signal outputted from the board connection information storing circuit 171 and the board connection information storing circuit 182, the CPU 131 of the main controlling unit 75 determines that both of the expansion controlling unit 77A and the expansion controlling unit 77B are connected if the obtained binary data is ‘100’. Then, the CPU 131 automatically causes to display (the image based on) the network related information outputted from the expansion controlling units 77A and 77B, the endoscope form detected image and the zoom control information at a predetermined position (any of the upper left, lower left, upper right and lower right on the screen) set on the setting screen shown in FIG. 8 to be described later.

(The image based on) the network related information, the endoscope form detected image, and the zoom control information may be outputted in a state adjusted by the CPU 131 in positions and image sizes so that the image and information are not overlapped by each other, or may be outputted with priority for overlapping one being outputted (for example, in a state in which the endoscope form detected image is displayed at the front).

The information and the like outputted from the expansion controlling units 77A and 77B may be set as hidden on the setting screen shown in FIG. 8 to be described later.

If the obtained binary data is ‘111’, the CPU 131 of the main controlling unit 75 determines that the board connection detecting signals from both of the board connection information storing circuit 171 and the board connection information storing circuit 182 cannot be detected, i.e., that neither the expansion controlling unit 77A nor the expansion controlling unit 77B is connected. Accordingly, the CPU 131 can causes to display neither (the image based on) the network related information, the endoscope form detected image nor the zoom control information outputted from the expansion controlling units 77A and 77B.

In the present embodiment, it is assumed that both of the expansion controlling units 77A and 77B are connected to the processor 4 as the controlling unit 77.

Now, processing performed by the CPU 131 of the main controlling unit 75 to detect (have detected) each board connected as the expansion controlling unit 77, when the processor 4 is switched off to on, or when the processor 4 is reset, will be described with reference to the flowchart shown in FIG. 2H.

The CPU 131 of the main controlling unit 75 detects whether either expansion board of the expansion controlling unit 77A and the expansion controlling unit 77B is connected as the expansion controlling unit 77 based on the board connection detecting signal outputted from the board connection information storing circuit 171 (and the board connection information storing circuit 182) (step DDDFLW1 shown in FIG. 2H). When the CPU 131 detects that none of the expansion boards is connected (step DDDFLW2 shown in FIG. 2H), the CPU 131 finishes the processing without displaying an image, information and the like outputted from the expansion controlling units 77A and 77B on a monitor and the like.

When the CPU 131 detects that any of the expansion boards is connected, it refers to the setting information corresponding to the connected expansion board among setting items in a ‘Board’ column in the setting screen shown in FIG. 8 to be described later and sets according to the setting information (step DDDFLW3 shown in FIG. 2H).

Then, the CPU 131 detects whether an input for turning on or off the display of the information or the image related to the connected expansion board has been done in the operating device or not (step DDDFLW4 and step DDDFLW5 shown in FIG. 2H).

In a case where an input for turning on the display of the information or the image related to the connected expansion board has been done in the operating device, the CPU 131 controls to display the information or the image (step DDDFLW6 shown in FIG. 2H). In a case where an input for turning off the display of the information or the image outputted from the connected expansion board has been done in the operating device, the CPU 131 controls to delete the information or the image (step DDDFLW7 shown in FIG. 2H).

The processing from step DDDFLW4 to step DDDFLW7 in the procedure described as the processing shown in FIG. 2H refers to the processing performed when a key to which any one of a ‘UPD’ function, a ‘ZScale’ function, and a ‘NET’ function is allocated, is operated in the operating device.

FIG. 3A to FIG. 3E show outlined configurations of (connectable) peripheral devices that can be connected to the processor 4. The peripheral devices that can be connected to the processor 4 includes appliance only supporting the display size (output size) of 4:3 and appliance that can support both of the display sizes (output size) of 16:9 and 4:3 to be described later. An example of each of the display size is shown in FIG. 4. Appliance such as a filing device that can record a signal (image) to be inputted among all units of appliance shown from FIG. 3A to FIG. 3E is adopted as an image recording unit, and appliance such as a monitor that can display a signal (image) to be inputted is adopted as a display unit.

A monitor 201A, a printer 202A, a VTR 203A, a filing device 204 and a photographing device 205A among the peripheral devices shown in FIG. 3A can perform at least one of the inputting/outputting, recording and displaying of analog signals in the SDTV system. Then, the peripheral device shown in FIG. 3A is connected to the image processing unit 72 via the signal line 111Sa and also to the SIO 142 and the PIO 143 of the main controlling unit 75.

A monitor 201B1, a printer 202B1, a VTR 203B1, a filing device 204B1 and a photographing device 205B1 among the peripheral devices shown in FIG. 3B can perform at least one of the inputting/outputting, recording and displaying of analog signals in the HDTV system and can support only the display size of 4:3. A monitor 201B2, a printer 202B2, a VTR 203B2, a filing device 204B2 and a photographing device 205B2 among the peripheral devices shown in FIG. 3B can perform at least one of the inputting/outputting, recording and displaying of analog signals in the HDTV system and can support both of the display sizes of 16:9 and 4:3. Then, the peripheral device shown in FIG. 3B is connected to the image processing unit 72 via the signal line 111Ha and also to the SIO 142 and the PIO 143 of the main controlling unit 75.

A monitor 201C1, a printer 202C1, a VTR 203C1, a filing device 204C1, a photographing device 205C1, an endoscope form detecting device 206C1 and an ultrasonic device 207C1 among the peripheral devices shown in FIG. 3C can perform at least one of the inputting/outputting, recording and displaying of analog signals (digital signals) in the SDTV system and the HDTV system and can support only the display size of 4:3. A monitor 201C2, a printer 202C2, a VTR 203C2, a filing device 204C2, a photographing device 205C2, an endoscope form detecting device 206C2 and an ultrasonic device 207C2 among the peripheral devices shown in FIG. 3C can perform at least one of the inputting/outputting, recording and displaying of analog signals (or digital signals) in the SDTV system or the HDTV system and can support both of the display sizes of 16:9 and 4:3. Then, the peripheral devices shown in FIG. 3C are connected to the image processing unit 72 via the signal line 121a and also to the SIO 142 and the PIO 143 of the main controlling unit 75. The peripheral devices shown in FIG. 3C can be connected to the controller 164 of the expansion controlling unit 77A by connection to the signal line denoted by E1 in the figure.

A printer 202D1, a filing device 204D1, a photographing device 205D1, an optical recording device 208D1 and an HID 209D1 among the peripheral devices shown in FIG. 3D can perform at least one of the inputting/outputting, recording and displaying via an USB (Registered Trademark) interface and can support only the display size of 4:3. A printer 202D2, a filing device 204D2, a photographing device 205D2, an optical recording device 208D2 and an HID 209D2 among the peripheral devices shown in FIG. 3D can perform at least one of the inputting/outputting, recording and displaying via a USB (Registered Trademark) interface and can support both of the display sizes of 16:9 and 4:3. The peripheral devices shown in FIG. 3D can be connected to the controller 164 of the expansion controlling unit 77A by connection to the signal line denoted by E2 in the figure. The optical recording devices 208 D1 and 208D2 are formed by any of the MO, the DVD (including Blue-lay and HDDVD), the CD±R/W or the like. Each of the HIDs 209D1 and 209D2 is an operating device including a keyboard, a mouse or a wheel.

A printer 202E1, a filing device 204E1, a photographing device 205E1, and an optical recording device 208E1 among the peripheral devices shown in FIG. 3E can perform at least one of the inputting/outputting, recording and displaying via the Ethernet (Registered Trademark) interface and can support only the display size of 4:3. A printer 202E2, a filing device 204E2, a photographing device 205E2, and an optical recording device 208E2 among the peripheral devices shown in FIG. 3E are connected to the processor 4 via a network by the network communication function of the expansion controlling unit 77A, can perform at least one of the inputting/outputting, recording and displaying via the Ethernet (Registered Trademark) interface, for example and can support both of the display sizes of 16:9 and 4:3. The peripheral devices shown in FIG. 3E can be connected to the HUB 162 of the expansion controlling unit 77A via the signal line 162a. The optical recording devices 208 E1 and 208 E2 are formed by any of the MO, the DVD, CD±R/W and the like.

The peripheral devices 200X1, 200X2 and 200X3 as any one of the peripheral devices may communicate with the controller 144 of the main controlling unit 75 via the signal line 144a by using The Token Passing protocol including the Token Ring, the FDDI, the Circlink, or the Arcnet.

Each of the peripheral devices 200X1, 200X2, and 200X3 has almost the same configuration as that of the controller 144 and the memory 145 of the main controlling unit 75. Accordingly, the peripheral device 200X1 is mainly described below for the simplicity of the description.

The peripheral device 200X1 has a controller IC 210A with almost the same configuration as that of the controller 144, a memory 211A with almost the same configuration of the memory 145, and a real-time clock that is not shown in the figure. The controller IC 210A can exchange various types of data including an image, endoscope related information, a log of program operations, maintenance information, setting information of the processor 4 and the other peripheral devices with the controller 144. The controller IC 210A stores the various types of data in the memory 211A.

The memory 211A has a shared region including two or more fixed regions into which the latest setting information on each of the peripheral devices 200X1, 200X2, and 200X3 is stored as shown in FIG. 3G and a log region in which transition (update history) of the contents of the shared region is chronologically stored as shown in FIG. 3H.

The shared region of the memory 211A is a region in which pieces of data including the setting information on the peripheral device 200X1 and each device connected to the peripheral device 200X1 are stored in fixed regions respectively. The pieces of data are to be referred to by each device.

Each piece of information stored in the log region of the memory 211A may be such that old date-time information is overwritten by the new one to keep the latest information left when information is written in all regions, or the information is unavailable to be written when information is written in all the regions. The data form of the information to be stored in the shared region and the log region of the memory 211A may be any of the ASCII data, the JIS data, and the binary data.

The peripheral device 200X1 transmits the latest setting information related to the peripheral device 200X1 stored in the shared region and date-time information of the transmission to each device connected to the peripheral device 200X1.

Each controller of the other devices connected to the peripheral device 200X1 periodically updates data related to the peripheral device 200X1 in the memory by storing the latest data related to the peripheral device 200X1 together with the received date-time information in a fixed region in the peripheral device 200X1 in each memory that is connected to each controller. Here, the Token Passing protocol is used in communication between the peripheral device 200X1 and each device. That enables the latency time of transmission for each device to be defined, thus, enables real-time processing on data. Thus, the pieces of information on the shared regions for all devices connected to the processor 4 are the same value.

For the date-time information (updated date and time) in the shared region in the memory 211A, a fixed region of each device is prepared as shown in FIG. 3G. Alternatively, one storing region may be prepared and the storing region may be shared when the updated timings of all fixed region are set as the same date and time.

Usage of data in the shared region in the memory 211A in the case where the peripheral device 200X1 among the peripheral devices connected to the processor 4 is switched from off to on, or when the peripheral device 200X1 is reset will be described with reference to the flowchart shown in FIG. 3I.

When it is detected that the peripheral device 200X1 is switched from off to on or that the peripheral device 200X1 is reset, the controller IC 210A of the peripheral device 200X1 receives information on the shared region of the other device by using the Token Passing protocol and stores the information in the memory 211A, and also refers to the date-time information of the peripheral device 200X1 stored in the shared region in the memory 211A (step CCCFLW1 shown in FIG. 3I).

Then, the controller IC 210A of the peripheral device 200X1 calculates the difference between the date and time recorded as the date-time information in the shared region in the memory 211A on the peripheral device 200X1 and the current date and time.

When it is detected that the difference between the date and time recorded as date-time information on the peripheral device 200X1 and the current date and time shown by the real-time clock (not shown) is within a predetermined period (step CCCFLW2 shown in FIG. 3I), the controller IC 210A updates the setting of the peripheral device 200X1 by reading the setting information of the peripheral device 200X1 recorded in the shared region in the memory 211A, and finishes the processing (step CCCFLW3 shown in FIG. 3I).

When it is detected that the difference between the date and time recorded as date-time information on the peripheral device 200X1 and the current date and time shown by the real-time clock (not shown) is over a predetermined period (step CCCFLW2 shown in FIG. 3I), the controller IC 210A initializes the setting of the peripheral device 200X1 and transmits the initialized setting information to the devices other than the peripheral device 200X1 (the peripheral device 200X2, and the peripheral device 200X3), and also updates the setting information recorded in the shared region in the memory 211A as an initialized value (step CCCFLW4 shown in FIG. 3I).

Even when the device is used as the power source of the device is repeatedly switched on and off according to the processing of the flowchart shown in FIG. 3I and if the difference is within the predetermined period (for example, 30 minutes have passed in the same day), the peripheral device 200X1 can immediately set itself by using the setting information stored in the shared region of the other device. That enables the user to immediately perform examination. As a result, the examination becomes more efficient and needs shorter time. Those effects are particularly remarkable when setting related to light-controlling of a peripheral device that takes time before the initial setting or the state in use, for example, the light equipment 3 is done, when setting related to the selection of the filter is done, when setting the endoscope related information in each filing device (except for a filing device 204A) mentioned above is done, or when setting output of an electric knife (not shown) is done.

The image compressing unit 73 of the processor 4 specifically has the configuration shown in FIG. 5 (and described below). Each unit of the image compressing unit 73 can be controlled by the CPU 131 of the main controlling unit 75 or (and) the CPU 151 of the expansion controlling unit 77 A (77B).

An image to be outputted (still image) that is outputted via the signal line 125a of the image processing unit 72 is temporality stored in the memory 222 via the controller 221 and also outputted to the selector 223 at timing according to clock signals of 100 MHz generated by the SSG 123. The memory 222 is adopted to store two or more still images.

When a thumbnail image is to be generated according to the determination on whether the thumbnail image is to be generated, the image to be outputted, which is inputted to the selector 223, is outputted to the selector 225 via the thumbnail image generating circuit 224. When a thumbnail image is not to be generated, the image to be outputted is outputted to the selector 225 without passing through the thumbnail image generating circuit 224.

The thumbnail image generating circuit 224 generates and outputs the image to be outputted reduced by ½ to 1/16, for example, as a thumbnail image based on the image to be outputted that is outputted from the selector 223. The size of the reduced image is set by the CPU 131 of the main controlling unit 75.

The image to be outputted that is inputted to the selector 225 is selectively outputted to the selector 226 according to which of the reduced image (thumbnail image) and the images that have not be reduced is to be outputted.

When YUV conversion is to be performed according to the determination on whether the YUV conversion is to be performed or not, the image to be outputted that is inputted to the selector 226 is outputted to the selector 228 via a YUV converting circuit 227. When YUV conversion is not to be performed (for example, the image is left as an RGB image), the image to be outputted is outputted to the selector 228 without passing through a YUV converting circuit 227.

The YUV converting circuit 227 performs the YUV (YCrCb) conversion on the image to be outputted that is outputted from the selector 226 and outputs the image to the selector 228.

The image to be outputted that is inputted to the selector 228 is selectively outputted to the selector 229 according to the determination on whether the image to be outputted that is subjected to the YUV conversion is to be outputted or not.

The image to be outputted that is inputted to the selector 229 is outputted to the selector 231 via a compression/conversion circuit 230 when compression/conversion is to be performed according to the determination on whether the compression/conversion is to be performed or not. When the compression/conversion is not to be performed, the image is to be outputted to the selector 231 without passing through the YUV converting circuit 230.

The compression/conversion circuit 230 encodes (or converts) the image to be outputted that is outputted from the selector 226 into any of the formats of the JPEG, the JPEG2000, the TIFF, and the BMP and outputs the image to be outputted to the selector 231.

The image to be outputted that is inputted to the selector 231 is selectively outputted to the controller 232 according to the determination on whether the image to be outputted subjected to the compression/conversion is to be performed or not.

The image to be outputted that is outputted from the selector 231 is temporarily stored in the memory 233 via the controller 232, subjected to the conversion according to the interface of the bus bridge 163 of the expansion controlling unit 77A based on the control by the CPU 131 of the main controlling unit 75, and then outputted to the CPU 151 via the bus bridge 163. The image to be outputted that is outputted from the selector 231 may be temporarily stored in the memory 233 via the controller 232 and directly outputted to the controller 241 of the image decompressing unit 74. The memory 222 and the memory 233 may be different address regions in the same memory.

The selector 234 selectively outputs a clock signal to a size change circuit 235 according to the system applied to the image to be outputted that is inputted via the signal line 124a among clock signals of 13.5 MHz and clock signals of 74 MHz which are generated at the SSG 123.

The image to be outputted (moving image) that is outputted via the signal line 124a of the image processing unit 72 is subjected to the reduction by the size change circuit 235 and the YUV conversion by the YUV converting circuit 236, encoded by the moving image encoding circuit 237, and then outputted to the CPU 151 via the bus bridge 163 of the expansion controlling unit 77A. The encoding performed by the moving image encoding circuit 237 may be any of the AVI form, the MPEG (MPEG2 or MPEG4) form, H.264 form and the WMV form.

The compression performed on a still image that is inputted to the compression/conversion circuit 230 and encoding performed on the moving image that is inputted to the moving image encoding circuit 237 may be performed in parallel. Processing performed on each unit of the image compressing unit 73 may be performed at timing to synchronize any of the ODD/EVEN determining signal outputted from the SSG 123 of the image processing unit 72 or a vertical synchronizing signal and a horizontal synchronizing signal.

The image decompressing unit 74 of the processor 4 is specifically adopted as shown in FIG. 6 (and described below). Each unit of the image decompressing unit 74 can be controlled by the CPU 131 of the main controlling unit 75, the CPU 151 of the expansion controlling unit 77 A (77B) or (and) the like.

The image (still image) to be outputted that is outputted from the controller 232 via the bus bridge 163 is temporarily stored in the memory 242 via the controller 241 and also outputted to the selector 243 based on the control by the CPU 131 of the main controlling unit 75.

When the image has been subjected to the compression (encoding) according to the encoding performed on the image to be outputted (any of the JPEG, the JPEG2000, the TIFF, the BMP, incompressible and the like), the image to be outputted that is inputted to the selector 243 is outputted to the selector 245 via a decompression/conversion circuit 244. When the image has not been subjected to the compression (encoding), the image to be outputted is outputted to the selector 245 without passing through the decompression/conversion circuit 244.

The decompression/conversion circuit 244 performs the decompression/conversion on the image to be outputted that is outputted from the selector 243 according to the format of the image to be outputted and outputs the image to be outputted to the selector 245.

The image to be outputted that is inputted to the selector 245 is selectively outputted to the selector 246 according to the determination on whether the image to be outputted that has been subjected to the decompression/conversion is to be outputted or not.

When RGB conversion is to be performed on the image to be outputted that is inputted to the selector 246, for example, according to the determination on whether the RGB conversion is to be performed or not, the image to be outputted is outputted to the selector 248 via an RGB conversion circuit 247. When the RGB conversion is not to be performed on the image to be outputted, the image to be outputted is outputted to the selector 248 without passing through the RGB conversion circuit 247.

The RGB conversion circuit 247 performs RGB conversion on the image to be outputted that is to be outputted from the selector 246 and outputs the image to be outputted to the selector 248.

The image to be outputted that is inputted to the selector 248 is selectively outputted to the selector 249 according to the determination on whether the image to be outputted that has been subjected to the RGB conversion is to be outputted or not.

When either a thumbnail image or a multi image is to be generated, for example, according to the determination on whether the thumbnail image or the multi image is to be generated or not, the image to be outputted that is inputted to the selector 249 is outputted to the selector 251 via the thumbnail/multi-image generating circuit 250. When neither a thumbnail image nor a multi image is to be generated, the image to be outputted is outputted to the selector 251 without passing through the thumbnail/multi-image generating circuit 250.

The thumbnail/multi-image generating circuit 250 generates an image to be outputted reduced by ½ to 1/16, for example, as a thumbnail image according to each image to be outputted that is outputted from the selector 249, and also generates and outputs the multi image, for which a thumbnail image is listed, as an image to be outputted. The size of the reduced images is set by the CPU 131 or the like of the main controlling unit 75.

The image to be outputted that is inputted to the selector 251 is selectively outputted to the synchronous circuit 252 at timing according to a clock signal of 100 MHz that is generated by the SSG 123 according to the determination on whether the multi image is to be outputted or not.

The synchronous circuit 252 outputs the image to be outputted which is to be outputted from the selector 251 to the composition/masking processing circuits 108H and 108S at timing according to any of the clock signal of 13.5 MHz, the clock signal of 74 MHz, and the clock signal of 100 MHz, which are generated by the SSG 123, for example. Connection between the synchronous circuit 252 and the composition/masking processing circuits 108H and 108S is denoted by F1 in the figure.

Now, the inner configuration of the synchronous circuit 252 will be described.

The synchronous circuit 252 includes the memory controller 252 A and the memories 252B and 252C as shown in FIG. 2G. The clock signal of 100 MHz that is generated by the SSG 123 is inputted to each unit of the synchronous circuit 252.

The memory controller 252 A controls inputting/outputting of the memories 252B and 252C based on the clock signal and the like that is outputted from the SSG 123 and the control of the main controlling unit 75.

The memory 252B is adopted as an FIFO memory. The memory 252B can store the image to be outputted that is outputted from the selector 251 for one frame (or for one line) based on the clock signal of 74 MHz that is generated by the SSG 123 and serially outputs the image to the composition/masking processing circuit 108H. The memory 252C is adopted as an FIFO memory. The memory 252C can store the image to be outputted that is outputted from the selector 251 for one frame (or for one line) based on the clock signal of 5 MHz that is generated by the SSG 123 and serially outputs the image to the composition/masking processing circuit 108S.

When the clock signal of 100 MHz that is to be inputted to each unit of the image decompressing unit 74 and each unit of the synchronous circuit 252 is replaced by the clock signal of 74 MHz, the moving image can be outputted to the image decompressing unit 74. In such a case, the image decompressing unit 74 may be adopted as a programmable circuit such as the FPGA, the DSP or a dynamic reconfigurable processor and be capable of switching the functions to serve as any of a circuit with a function of decompressing a still image and a circuit with a function of decompressing a moving image.

When the image decompressing unit 74 is adopted as a programmable circuit, the unit may be adopted to select the decompressing system (one from the JPEG, the JPEG2000, the TIFF, the BMP, the AVI, the MPEG, H.264 and the WMV) on the setting screen shown in FIG. 8 to be described later so that a block (firmware) according to the selection is to be downloaded. The block may be downloaded in any manner of that the block is downloaded by the CPU 151 of the expansion controlling unit 77A via the bus bridge 163, or downloaded from a ROM or the like (not shown) provided for the image decompressing unit 74. During the downloading of the block, an error message directing that the downloading is on the way may be displayed on the endoscope composite screen, or a predetermined LED (not shown) of the operating device may be lit (or flickered). When the block has been downloaded, a message directing the completion of the download may be displayed on the screen.

The selector 256 selectively outputs a clock signal according to the image to be outputted that is outputted from the moving image encoding circuit 237 among the clock signals of 13.5 MHz and 74 MHz which are generated by the SSG 123, via the bus bridge 163 to the size change circuit 255.

The image to be outputted (moving image) that is outputted from the moving image encoding circuit 237 via the bus bridge 163 is subjected to decoding according to encoding on the image to be outputted by the moving image decoding circuit 253, the RGB conversion by the RGB conversion circuit 254, the reduction by the size change circuit 255, and then outputted to the composition/masking processing circuits 108H and 108S. Connection between the size change circuit 255 and the composition/masking processing circuits 108H and 108S is denoted by F2 in the figure.

The decompression that is performed on a still image that is to be inputted to the decompression/conversion circuit 244 and the decoding that is performed on a moving image that is to be inputted to the moving image decoding circuit 253 may be performed in parallel. Processing performed on each unit of the image decompressing unit 74 may be performed at timing to synchronize any of the ODD/EVEN determining signal outputted from the SSG 123 of the image processing unit 72 or a vertical synchronizing signal and a horizontal synchronizing signal.

FIG. 7 shows an example of the endoscope composite image generated by the composition/masking processing circuit 108H or 108S. Each unit shown in FIG. 7 will be described from the item 1) to the item 27).

1) Endoscope Image 301

is always displayed when the endoscope 2A (or the endoscope 2B) is connected (hidden when the endoscope is not connected.)

is changed in the image size according to the operation and the like performed on an image size changing key and the like allocated to the operating device.

2) Endoscope Image 302

is displayed when the S freeze switch allocated to the operating device is operated.

3) Arrow Pointers 301a and 302a

are displayed in green and the like (that is easily distinguished from the color of the subject in the living body).

are displayed also to show relative position of the outputted SDTV image (for example, outputted via the signal line 111Sa) and the outputted HDTV image (for example, outputted via the signal line 111Ha).

can display, delete and change the direction of the distal end portion according to the key input performed on the keyboard 5 (for example, combinations of the SHIFT key and cursor keys ‘↑’, ‘↓’, ‘←’, ‘→’ keys).

can move on the screen in response to the cursor keys of the keyboard 5.

are hidden when a predetermined operation is performed on the keyboard 5 (or an operation on a key with a function of reporting the end of examination and the like).

either of the arrow pointers 301a and 302a can be selected according to a predetermined operation on keys of the keyboard 5, and the arrow pointers can be independently displayed, deleted and moved.

4) ID No. (Patient ID) 303

An item name (ID No.) is displayed before inputting data or when an operation on a key with a function of reporting the end of examination or the like is performed. The item name is automatically deleted when data is inputted from the keyboard 5 or the like and the input data up to 15 characters is displayed.

When data has not inputted and a cursor is moved in response to key input such as a cursor key and the like of the keyboard 5, the item name is deleted.

When the patient ID data is received from the peripheral device, the received ID data is displayed.

5) Name (Patient's Name) 304

The item name (Name) is displayed before inputting data or when an operation on a key with a function of reporting the end of examination or the like is performed. The item name is automatically deleted when data is inputted from the keyboard 5 or the like and the input data up to 20 characters is displayed.

When data has a space, a line feed is inserted at the space position. (For example, in FIG. 7, as there is a space between ‘yamada’ and ‘gentle’, ‘gentle’ is displayed in a line below that of ‘yamada’.)

When data has not been inputted and a cursor is moved in response to key input performed on a cursor key or the like of the keyboard 5, the item name is deleted.

When patient's name data is received from the peripheral device, the received patient's name data is displayed.

6) Sex (Patient's Name) 305

When data has not been inputted or when an operation is performed on a key with a function of reporting the end of examination, the item name (Sex) is displayed. The item name is automatically deleted according to the data inputted by the keyboard 5 or the like, and input data up to a character is displayed.

When data has not been inputted and a cursor is moved in response to key input performed on a cursor key or the like of the keyboard 5, the item name is deleted.

When the patient's name data is received from the peripheral device, the received patient's name data is displayed.

7) Age (Patient's Age) 306

When data has not been inputted or when an operation is performed on a key with a function of reporting the end of examination, the item name (Age) is displayed. The item name is automatically deleted according to the data inputted by the keyboard 5 or the like, and input data up to three characters is displayed.

When D.O. Birth is inputted, the CPU 131 calculates the age and the age is automatically inputted and displayed.

When data has not been inputted and a cursor is moved in response to key input performed on a cursor key or the like of the keyboard 5, the item name is deleted.

When the patient's age data is received from the peripheral device, the received patient's age data is displayed.

8) D. O. Birth (Birth Date of the Patient) 307

When data has not been inputted or when an operation is performed on a key with a function of reporting the end of examination, the item name (D. O. Birth) is displayed. The item name is automatically deleted according to the data inputted by the keyboard 5 or the like, and input data is displayed.

When data has not been inputted and a cursor is moved in response to key input performed on a cursor key or the like of the keyboard 5, the item name is deleted.

D. O. Birth can be inputted by up to eight characters in the western calendar and up to seven characters in the Japanese calendar (M: Meiji, T: Taisho, S: Showa, H: Heisei). The display form may be set on the setting screen of the processor 4.

Data on the patient's birth date is received from the peripheral device, the received data on the patient's birth date is displayed.

9) Time Information 308

Current date and time and a stop watch are displayed. The date and time can be set on the setting screen of the processor 4.

Time information can be omitted from the display. The lower two digits may displayed each for the date and time so as not to overlap the endoscope image when the information is omitted from the display.

The stopwatch may be displayed at different position according to the system of image to be outputted (SDTV or HDTV).

The date may be hidden when the stop watch is operating in the SDTV output. For example, the stop watch is displayed in the display form of HH″ MM′ SS (time″ minute′ second).

When freeze is performed by the freeze key, the freeze is not performed (except for the stop watch).

10) SCV309

The item (‘SCV:’) and the count for the Release operation in the photographing device (any of the photographing devices 205A, 205B1, 205B2, 205C1, 205C2, 205D1, 205D2, 205E1, and 205E2) that is selected on the setting screen of the processor 4 are displayed. (The item and count are not displayed when the item and count are set to OFF on the setting screen of the processor 4)

When the communication with the photographing device is established, the count outputted from the photographing device is displayed. When the communication with the photographing device is not established, the count of the Release operation that is counted by the CPU 131 of the main controlling unit 75 is displayed.

11) CVP 310

When the communication with the printer (any of the printer 202A, 202B1, 202B2, 202C1, 202C2, 202D1, 202D2, 202E1 and 202E2) that is selected on the setting screen of the processor 4 is established, the item (‘CVP:’), the number of captures, the number of divisions, and a memory page are displayed.

12) D.F 311

When the communication with the filing device (any of the filing devices 204A, 204B1, 204B2, 204C1, 204C2, 204D1, 204D2, 204E1, and 204E2) that is selected on the setting screen of the processor 4 is established, the item (‘D.F:’) and the count of the Release operation are displayed. (The count is based on the count command that is outputted from the filing device.)

13) VTR 312

When the communication with the VTR (any of the VTRs 203A, 203B1, 203B2, 203C1, and 203C2) that is selected on the setting screen of the processor 4 is established and while a moving image is recorded or the moving image recorded in the VTR is played by the VTR, the VTR 312 is displayed.

14) PUMP 313

When the communication with a forward-water-feeding pump (not shown) is established and while the forward-water-feeding pump is driven, PUMP 313 is displayed.

15) Are for the Peripheral Device 314

The received data from the peripheral device such as error information is displayed up to 20 characters (ten characters/a line).

16) Physician (Physician's Name) 315

When data has not been inputted or when an operation is performed on a key with a function of reporting the end of examination and the like, the item name (Physician) is displayed (When an operation is performed on a key with a function of reporting the end of examination and the like, it may be deleted.) The item name is automatically deleted according to the data inputted by the keyboard 5 or the like, and input data is displayed up to 20 characters.

When data has not been inputted and a cursor is moved in response to key input performed on a cursor key or the like of the keyboard 5, the item name is deleted.

When physician's name data is received from the peripheral device, the received physician's name data is displayed.

17) Comment 316

When data has not been inputted, the item name (Comment) is displayed. (When an operation is performed on the key with a function of reporting the end of examination and the like, the comment may be displayed.) The item name is automatically deleted according to the data inputted by the keyboard 5 or the like, and input data is displayed up to 37 characters.

When the comment data is received from the peripheral device, the received comment data is displayed.

18) Endoscope Switch Information 317

Each function allocated to the operation switching section 28A (28B) of the endoscope 2A (2B) is displayed for each occasion of switching.

19) Endoscope Related Information 318

Information on the endoscope 2A (2B) stored in the memory 30A (30B) of the endoscope 2A (2B) is displayed.

20) Cursor 319

For example ‘I’ is displayed in the character inserting mode (when ‘INS’ or ‘Insert’ key of the keyboard 5 is turned off).

For example, a square filled with a predetermined color is displayed in the character overwriting mode (when ‘INS’ or ‘Insert’ key of the keyboard 5 is turned off).

For example, ‘I’ in a color different from that in the character inserting mode (light blue or the like) is displayed in the Alphabet inputting mode (when ‘Alphabet’ keys on the keyboard 5 are turned on).

When ‘CAPS LOCK’ key on the keyboard 5 is turned on, capital letters can be inputted.

When ‘CAPS LOCK’ key on the keyboard 5 is turned off, the cursor is displayed with its height in half of that displayed while ‘CAPS LOCK’ key is turned on and lower case letters can be inputted.

flickered.

21) Contrasts (C_{T}) 320A and 320 B

A contrast setting that is set by a contrast key allocated to the operating device is displayed. (Display example: ‘N’ Normal, ‘L’ Low, ‘H’ High, ‘4’ non-corrected)

22) Color Highlights (C_{E}) 321A and 321B

Setting for color highlights set by a color highlight key allocated to the operating device is displayed.

23) Hemoglobin Index (IHb) 322A and 322B

The IHb value in the case where the freeze switch is operated and a freeze image is outputted is displayed in IHb 322A. The IHb value in the case where the S freeze switch is operated and an S freeze image is displayed is displayed in the IHb 322B.

When no freeze direction is issued, ‘---’ is displayed.

When ‘AFI’ is displayed in the light source filter type 325 A or 325B to be described later, the contrasts need not be displayed.

24) Structure Highlighting (E_{H})/edge highlighting (E_{D}) 323A and 323B Setting of structure highlighting or edge highlighting that is set by the highlight key allocated to the operating device is displayed.

Either ‘E_{H}:A*’directing the structure highlighting A or ‘E_{H}:B*’ directing the structure highlighting B is displayed when structure highlighting is performed (* in each case denotes a numeral).

Any of the three types of ‘E_{D}:O’, ‘E_{D}:L’, ‘E_{D}:H’ or any of the three types of ‘E_{D}:L’, ‘E_{D}:M’, ‘E_{D}:H’ is displayed when edge highlighting is performed.

25) Enlargement Ratios 324A and 324B

Setting of electronic enlargement that is set by an electronic enlargement key allocated to the operating device is displayed.

The enlargement ratios 324A and 324B are displayed only when an endoscope having a CCD supporting electronic enlargement is connected to the processor 4.

26) Light Source Filter Types 325A and 325B

The type of a filter that is set to be used according to the observation among special optical filters of the light equipment 3 is displayed.

When a filter supporting general optical observation is set to be used (or no special optical filter is used), ‘Normal (or Nr)’ is displayed.

When a filter supporting narrow bandage optical observation is set to be used, ‘NBI’ is displayed.

When a filter supporting fluorescence observation is set to be used, ‘AFI’ is displayed.

When a filter supporting infrared rays observation is set to be used, ‘IRI’ is displayed.

27) Thumbnail Image 326

Up to four images (for thumbnail images) are displayed. (They may be set as display OFF, or may be deleted when a key or a switch allocated to the release function is inputted first after the key with a function of reporting the end of examination or the like is operated.)

In the description below, for the simplicity of the description, the elements of the items from the item 4) to the item 20), i.e., the elements from ID No. 303 to the cursor 319 are categorized in the group of observe information 300, the elements from the contrast 320A to the light source falter type 325A which are the information related on the endoscope image 301 are categorized in the group of image related information 301A, the elements from the contrast 320B to the light source filter type 325B which are the information related on the endoscope image 302 are categorized in the group of image related information 302A, and thumbnail images 326 are categorized in the group of thumbnail image 326A. The group of the image related information 302A shows the image related information that is related to the S freeze image only when the S freeze image is displayed as the endoscope image 302.

FIG. 8 shows an example of the setting screen of the processor 4. Now, functions related to the items which can be set on the setting screen and functions related to the items will be described. The setting screen of the processor 4 shown in FIG. 8 is generated by the graphic circuit 106S (106H) of the image processing unit 72.

In the item ‘thumbnail’, whether a thumbnail image is to be created or not can be set. When ‘ON’ is set for the item ‘thumbnail’, the CPU 131 of the main controlling unit 75 controls the selectors 223 and 225, and outputs the image to be output via the thumbnail image generating circuit 224 of the image compressing unit 73. When ‘OFF’ is set for the item ‘thumbnail’, the CPU 131 of the main controlling unit controls the selectors 223 and 225, and outputs the image to be output without passing it through the thumbnail image generating circuit 224 of the image compressing unit 73.

In the item ‘Scope Switch’, functions of allocating the CPU 131 of the main controlling unit to the operation switching section 28A of the endoscope 2A which serves as an operating device and each switch of the operation switching section 28B of the endoscope 2B which serves as an operating device can be set. Each function that can be allocated to each of the above mentioned switch will be detailed later.

In the item ‘Foot Switch’, functions of allocating the CPU 131 of the main controlling unit 75 to each switch of the foot switch 6 which serves as an operating device can be set. Each function that can be allocated to each of the above mentioned switch will be detailed later.

In the item ‘Keyboard’, functions of allocating the CPU 131 of the main controlling unit to one or more keys among respective keys on the keyboard 5 which serves as an operating device can be set. Each function that can be allocated to each of the above mentioned switch will be detailed later.

In the item ‘Front Panel’, functions of allocating the CPU 131 of the main controlling unit 75 to one or more keys among respective keys on the front panel 76 which serves as an operating device can be set. Each function that can be allocated to each of the above mentioned switch will be detailed later.

In the items ‘Release1’, ‘Release2’, ‘Release3’ and ‘Release4’ in the column ‘SDTV’, recording conditions and appliances to record the still image can be set by using some of the functions related to recording of a still image in the SDTV system among the functions which can be allocated to any of the items ‘Scope Switch’, ‘Foot Switch’, ‘Keyboard’ and ‘Front Panel’, each of which is the sub-item shown below. What can be set by each sub-item of the items ‘Release1’, ‘Release2’, ‘Release3’ and ‘Release4’ in the column ‘SDTV’ are the same. Thus, only the sub-items of ‘Release1’ will be described below.

For ‘Peripheral Device’, which is a sub-item of the item ‘Release1’, an appliance to record a still image in the SDTV system can be set. The appliance to record the image may be any one of all filing devices (except for the filing devices 204B1 and 204B2), all photographing devices (except for the photographing devices 205B1 and 205B2), all optical recording devices, the PC card 167 and the memory card 168 shown from FIG. 3A to FIG. 3E. When ‘OFF’ is set for the item ‘Peripheral Device’, it is set that the appliance does not record a still image in the SDTV system even if there is no appliance to record an image, i.e., a key or a switch with the function of ‘Release1’ allocated is operated.

For ‘Encode’, which is a sub-item of the item ‘Release1’, a format to be used in recording a still image in the SDTV system can be set. The format that can be set here is any of the JPEG, the JPEG2000, the TIFF, or the BMP. When any of those formats is selected and set for the item ‘Encode’, the CPU 131 of the main controlling unit 75 controls the selectors 229 and 231, and outputs the image to be outputted via the compression/conversion circuit 230 of the image compressing unit 73. When ‘OFF’ is selected in the item ‘Encode’, the CPU 131 of the main controlling unit controls the selectors 229 and 231, and outputs the image to be outputted without passing it through the compression/conversion circuit 230 of the image compressing unit 73.

For ‘Signal’, which is a sub-item of the item ‘Release1’, the signal format of the image to be outputted can be set to either a YCrCb signal or an RGB signal. When ‘YCrCb’ is selected and set in the item ‘Signal’, the CPU 131 of the main controlling unit 75 controls the selectors 226 and 228 and outputs the image to be outputted via the YUV converting circuit 227 of the image compressing unit 73. When ‘RGB’ is selected in the item ‘Signal’, the CPU 131 of the main controlling unit 75 controls the selectors 226 and 228 and outputs the image to be outputted without passing it through the YUV converting circuit 227 of the image compressing unit 73.

For ‘Format’, which is a sub-item of the item ‘Release1’, the format of the YCrCb signal or the RGB signal which has been set in the item ‘Signal’ can be set. One or more of the formats of 4:2:0, 4:1:1, 4:2:2, 4:4:4, Sequential, Spectral Selection (frequency divided type), Successive Approximation (approximation accuracy improving type), DPCM (reversible type), Interleave, and Non-Interleave can be set. When any of those formats is selected and set in the item ‘Format’, the CPU 131 of the main controlling unit 75 causes the compression/conversion circuit 230 of the image compressing unit 73 to perform the compression/conversion according to the format. When ‘OFF’ is selected in the item ‘Format’, the CPU 131 of the main controlling unit 75 does not change the format to the YCrCb signal or the RGB signal that has been set in the sub-item ‘Signal’ of the item ‘Release1’ in the column ‘SDTV’.

For ‘Dot’, which is a sub-item of the item ‘Release1’, the quantizing accuracy of the YCrCb signal (component) or the RGB signal (component) that has been set in the sub-item ‘Signal’ of the item ‘Release1’ in the column ‘SDTV’ can be set as the number of dots of either eight bits or ten bits. Then, the CPU 131 of the main controlling unit causes the compression/conversion circuit 230 of the image compressing unit 73 to perform the compression/conversion by assuming that a signal to be inputted (component) has been quantized by the number of dots.

For ‘Level’, which is a sub-item of the item ‘Release1’, a level of compressing the image to be outputted can be set. The compression level can be selected from three levels of ‘High’ directing a high image quality and a big image size, ‘Normal’ directing an image quality lower and an image size smaller than those set in ‘High’, and ‘Low’ directing an image quality still lower and an image size still smaller than those set in ‘Normal’. Then, the CPU 131 of the main controlling unit 75 causes the compression/conversion circuit 230 of the image compressing unit 73 to perform the compression/conversion according to any of the three levels. In the case of the JPEG format, for example, each setting of the ‘High’, ‘Normal’ and ‘Low’ can be realized when a preset quantization table, a Huffman table or the like is used.

The items ‘Encode’, ‘Signal’, ‘Format’, ‘Dot’, and ‘Level’ among the items in the column ‘SDTV’ are enabled (settings can be changed) only when any of the filing devices shown in FIG. 3D and FIG. 3E, the photographing devices shown in FIG. 3D and FIG. 3E, the optical recording devices shown in FIG. 3D and FIG. 3E, the PC card 167 and the memory card 168 is selected in the sub-item ‘Peripheral Device’ of the item ‘Release’ in the column ‘SDTV’. When those items are disabled (setting cannot be changed), they are displayed in dark grey, for example.

In the items ‘Release1’, ‘Release2’, ‘Release3’, and ‘Release4’ in the column ‘HDTV’, recording conditions and appliances to record the still image can be set by using some of the functions related to recording of a still image in the HDTV system among the functions which can be allocated to any of the items ‘Scope Switch’, ‘Foot Switch’, ‘Keyboard’ and ‘Front Panel’, each of which is the sub-item shown below. What can be set by each sub-item of the items ‘Release1’, ‘Release2’, ‘Release3’ and ‘Release4’ in the column ‘HDTV’ are the same. Thus, only the sub-items of ‘Release1’ will be described below.

For ‘Peripheral Device’, which is a sub-item of the item ‘Release1’, an appliance to record a still image in the HDTV system can be set. The appliance to record the image may be any one of all filing devices shown from FIG. 3A to FIG. 3E (except for the filing devices 204A), all photographing devices (except for the photographing devices 205A), all optical recording devices, the PC card 167 and the memory card 168. When ‘OFF’ is set for the item ‘Peripheral Device’, it is set that the appliance does not record a still image in the HDTV system even if there is no appliance to record an image, i.e., a key or a switch with the function of ‘Release1’ allocated is operated.

For ‘Encode’, which is a sub-item of the item ‘Release1’, a format to be used in recording a still image in the HDTV system can be set. The format that can be set here is any of the JPEG, the JPEG2000, the TIFF, or the BMP. When any of those formats is selected and set for the item ‘Encode’, the CPU 131 of the main controlling unit controls the selectors 229 and 231, and outputs the image to be outputted via the compression/conversion circuit 230 of the image compressing unit 73. When ‘OFF’ is selected in the item ‘Encode’, the CPU 131 of the main controlling unit controls the selectors 229 and 231, and outputs the image to be outputted without passing it through the compression/conversion circuit 230 of the image compressing unit 73.

For ‘Signal’, which is a sub-item of the item ‘Release1’, the signal format of the image to be outputted can be set to either a YCrCb signal or an RGB signal. When ‘YCrCb’ is selected and set in the item ‘Signal’, the CPU 131 of the main controlling unit 75 controls the selectors 226 and 228 and outputs the image to be outputted via the YUV converting circuit 227 of the image compressing unit 73. When ‘RGB’ is selected in the item ‘Signal’, the CPU 131 of the main controlling unit controls the selectors 226 and 228 and outputs the image to be outputted without passing it through the YUV converting circuit 227.

For ‘Format’, which is a sub-item of the item ‘Release1’, the format of the YCrCb signal or the RGB signal which has been set in ‘Signal’, which is a sub-item of the item ‘Release’ in the column ‘SDTV’ can be set. One or more of the formats of 4:2:0, 4:1:1, 4:2:2, 4:4:4, Sequential, Spectral Selection (frequency divided type), Successive Approximation (approximation accuracy improving type), DPCM (reversible type), Interleave, and Non-Interleave can be set. When any of those formats is selected and set in the item ‘Format’, the CPU 131 of the main controlling unit 75 causes the compression/conversion circuit 230 of the image compressing unit 73 to perform the compression/conversion according to the format. When ‘OFF’ is selected in the item ‘Format’, the CPU 131 of the main controlling unit does not change the format to the YCrCb signal or the RGB signal that has been set in the sub-item ‘Signal’ of the item ‘Release1’ in the column ‘HDTV’.

For ‘Dot’, which is a sub-item of the item ‘Release1’, the quantizing accuracy of the YCrCb signal (component) or the RGB signal (component) that has been set in the sub-item ‘Signal’ of the item ‘Release1’ in the column ‘SDTV’ can be set as the number of dots to either eight bits or ten bits. Then, the CPU 131 of the main controlling unit causes the compression/conversion circuit 230 of the image compressing unit 73 to perform the compression/conversion by assuming that a signal to be inputted (component) has been quantized by the number of dots.

For ‘Level’, which is a sub-item of the item ‘Release1’, a level of compressing the image to be outputted can be set. The compression level can be selected from three levels of ‘High’ directing a high image quality and a big image size, ‘Normal’ directing an image quality lower and an image size smaller than those set in ‘High’, and ‘Low’ directing an image quality still lower and an image size still smaller than those set in ‘Normal’. Then, the CPU 131 of the main controlling unit 75 causes the compression/conversion circuit 230 of the image compressing unit 73 to perform the compression/conversion according to any of the three levels described above. In the case of the JPEG format, for example, each setting of the ‘High’, ‘Normal’ and ‘Low’ described above can be realized when a preset quantization table, a Huffman table or the like is used.

The items ‘Encode’, ‘Signal’, ‘Format’, ‘Dot’, and ‘Level’ among the items in the column ‘HDTV’ are enabled (settings can be changed) only when any of the filing devices shown in FIG. 3D and FIG. 3E, the photographing devices shown in FIG. 3D and FIG. 3E, the optical recording devices shown in FIG. 3D and FIG. 3E, the PC card 167 and the memory card 168 is selected in the sub-item ‘Peripheral Device’. When those items are disabled (setting cannot be changed), they are displayed in dark grey, for example.

Each item in the column ‘SDTV’ and the column ‘HDTV’ is not limited to be set on a setting screen shown in FIG. 8 by a user. A predetermined item may be set as predetermined content when a predetermined device is connected to the processor 4 and the predetermined device is selected in the item ‘Peripheral device’ in the column ‘SDTV’ and the column ‘HDTV’.

In the items ‘NETWORK’, ‘UPD’, and ‘ZOOM Controller’ in the column ‘Board’, setting related to the expansion controlling unit 77 can be set.

When the expansion controlling unit 77A is connected as the expansion controlling unit 77, whether (the image based on) the network related information that is outputted from the expansion controlling unit 77A is to be displayed or not and the display position of (the image based on) the network related information can be set in the item ‘NETWORK’.

When the expansion controlling unit 77B having some functions of the endoscope form detecting device is connected as the expansion controlling unit 77, whether the endoscope form image that is outputted from the expansion controlling unit 77B is to be displayed or not and the display position of the endoscope form image can be set in the item ‘UPD’.

When the expansion controlling unit 77B having a zoom controlling function is connected as the expansion controlling unit 77, whether the zoom control information that is outputted from the expansion controlling unit 77B is to be displayed or not and the display position of the zoom control information can be set in the item ‘ZOOM Controller’.

Each of the items ‘NETWORK’, ‘UPD’ and ‘ZOOM Controller’ has the items ‘PinP’ and ‘Position’ as a sub-item.

When ‘ON’ is set for the ‘PinP’, which is a sub-item of the item ‘NETWORK’, (the image based on) the network related information as mentioned above is displayed by the PinP. When ‘OFF’ is set, (the image based on) the network related information is displayed. The ‘ON’ or ‘OFF’ is not limited to be set on the setting screen as shown in FIG. 8 and it may be set in response to the operation performed on a key or a switch with a function of ‘NET’ to be described below allocated is operated.

For ‘Position’, which is a sub-item of the item ‘NETWORK’, the display position of (the image based on) the network related information that is displayed by PinP can be selected from the upper left, the lower left, the upper right, and the lower right.

When ‘ON’ is set for ‘PinP’, which is a sub-item of the item ‘UPD’, the endoscope form detected image is displayed by PinP. When ‘OFF’ is set, the endoscope form detected image is hidden. The ‘ON’ or ‘OFF’ is not limited to be set on the setting screen as shown in FIG. 8 and it may be set in response to the operation performed on a key or a switch with a function of ‘UDP’ to be described below allocated is operated.

For ‘Position’, which is a sub-item of the item ‘UDP’, the display position of endoscope from detected image that is displayed by PinP can be selected from the upper left, the lower left, the upper right, and the lower right.

When ‘ON’ is set for ‘PinP’, which is a sub-item of the item ‘ZOOM Controller’, the zoom control information is displayed by PinP. When ‘OFF’ is set, the zoom control information is hidden. The ‘ON’ or ‘OFF’ is not limited to be set on the setting screen as shown in FIG. 8 and it may be set in response to the operation performed on a key or a switch with a function of ‘ZScale’ to be described below allocated is operated.

For ‘Position’, which is a sub-item of the item ‘ZOOM Controller’, the display position of zoom control information displayed by PinP can be selected from the upper left, the lower left, the upper right, and the lower right.

In the items ‘SDTV’ and ‘HDTV’ in the column ‘Release Time’, a duration for displaying a still image after the release direction (recording direction) is issued can be set. The duration for displaying the still image may be selected from among 0.1, 0.5, 1, 2, 3, 4, 5, 6, 7, 8, and 9 seconds.

Each of the item ‘SDTV’ and the item ‘HDTV’ in the column ‘Release Time’ is not limited to be set on a setting screen shown in FIG. 8 by a user. A predetermined item may be set as predetermined content when a predetermined device is connected to the processor 4 and the predetermined device is selected in the item ‘Peripheral device’ in the column ‘Peripheral Device’.

In the item ‘Mon size’, the size for the screen to be displayed can be selected and set from 16:9 and 4:3.

In the item ‘Encryption’, whether the encryption and the decryption are to be performed by the encrypting circuit 170 of the expansion controlling unit 77A or not can be set.

For each of the items in the column ‘Movie Encode’, setting related to displaying, recording and the like of the moving image can be set.

In the item ‘SIZE’ in the column ‘Movie Encode’, the display size (aspect ratio) of the moving image can be selected and set from factors 1, 2/3, and 1/2. The CPU 131 of the main controlling unit 75 causes the size change circuit 235 of the image compressing unit 73 to perform image compression according to each of the factors. When a factor of 1 is selected from the factors, the CPU 131 of the main controlling unit 75 outputs the inputted moving image as it is without causing the size change circuit 235 to perform the compression.

In the item ‘Encode Type’ in the column ‘Movie Encode’, the format to be used in recording a moving image can be set. The format that can be set here is any of the AVI, the MPEG (MPEG 2 or MPEG4), H264, or the WMV. When any of the formats is selected and set in the item ‘Movie Encode’, the CPU 131 of the main controlling unit controls the moving image encoding circuit 237 of the image compressing unit 73 so that the moving is converted into the format selected in the item ‘Movie Encode’.

In the item ‘Signal’ in the column ‘Movie Encode’, the signal format of the moving image can be set to either a YCrCb signal or an RGB signal. When ‘YCrCb’ is selected and set in the item ‘Signal’, the CPU 131 of the main controlling unit 75 controls the YUV converting circuit 236 and outputs the moving image as YCrCb signals. When ‘RGB’ is selected and set in the item ‘Signal’, the CPU 131 of the main controlling unit 75 controls the YUV converting circuit 236 and outputs the moving image as RGB signals. The item ‘Signal’ in the column ‘Movie Encode’ may be automatically in a predetermined setting according to what is set for the item ‘Encode Type’.

In the item ‘Encode’ in the column ‘Movie Encode’, the type of the image to be encoded can be selected and set from the SDTV and the HDTV. When ‘SDTV’ is selected and set in the item ‘Encode’, the CPU 131 of the main controlling unit 75 controls the selector 124 of the image processing unit 72 so that outputting from the composition/masking processing circuit 108S is selected, and also controls the selector 234 of the image compressing unit 73 so that a clock signal of 13.5 MHz is selected. When ‘HDTV’ in the item ‘Encode’ is selected and set, the CPU 131 of the main controlling unit 75 controls the selector 124 of the image processing unit 72 so that outputting from the composition/masking processing circuit 108H is selected, and also controls the selector 234 of the image compressing unit 73 so that a clock signal of 74 MHz is selected.

In the item ‘Format’ in the column ‘Movie Encode’, the sampling system for the YCrCb signal or the RGB signal that has been set for the item ‘Signal’ in the column ‘Movie Encode’ can be set. Any of the sampling systems of 4:2:0, 4:1:1, 4:2:2, and 4:4:4 can be set. When any of the sampling systems is selected and set in the item ‘Format’, the CPU 131 of the main controlling unit 75 causes the moving image encoding circuit 237 of the image compressing unit 73 to perform encoding according to the sampling system. The item ‘Format’ in the column ‘Movie Encode’ may be automatically in a predetermined setting according to what is set for the item ‘Encode Type’.

For ‘Dot’ in the column ‘Movie Encode’, the quantizing accuracy of the YCrCb signal (component) or the RGB signal (component) that has been set in the item ‘Signal’ in the column ‘Movie Encode’ can be set as the number of dots of either eight bits or ten bits. Then, the CPU 131 of the main controlling unit causes the moving image encoding circuit 237 of the image compressing unit 73 to perform encoding by assuming that a signal to be inputted (component) has been quantized by the number of dots. The item ‘Dot’ in the column ‘Movie Encode’ may be automatically in a predetermined setting according to what is set for the item ‘Encode Type’.

In the item ‘Peripheral Device’ in the column ‘Movie Encode’, one or more appliance to record a moving image among peripheral devices which are connected to the processor 4 can be selected from all filing devices shown in FIG. 3D and FIG. 3E, all photographing devices shown in FIG. 3D and FIG. 3E, all optical recording devices shown in FIG. 3D and FIG. 3E, the PC card 167 and the memory card 168. When ‘OFF’ is set for the item ‘Peripheral Device’, it can be set that a moving image is not to be performed even if there is no appliance to record an image, i.e., a key or a switch with the function of recording the moving image allocated is operated.

In the item ‘Encryption’ in the column ‘Movie Encode’, whether the encryption is to be performed by the encrypting circuit 170 of the expansion controlling unit 77A on the moving image outputted from moving image encoding circuit 237 or not can be set.

In the item ‘Encode Level’ in the column ‘Movie Encode’, the maximum bit rate for the moving image can be set. The maximum bit rate can be selected from among three levels of ‘High’ directing a high image quality and a big image size, ‘Normal’ directing an image quality lower and an image size smaller than those set in ‘High’, and ‘Low’ directing an image quality still lower and an image size still smaller than those set in ‘Normal’. Then, the CPU 131 of the main controlling unit 75 causes the moving image encoding circuit 237 of the image compressing unit 73 to perform the encoding according to any of the three levels. The item ‘Encode Level’ in the column ‘Movie Encode’ may be automatically in a predetermined setting according to what is set for the item ‘Encode Type’.

Each item in the column ‘Movie Encode’ is not limited to be set by a user on the setting screen as shown in FIG. 8, and may be a predetermined item such as those automatically has a predetermined setting when a predetermined peripheral device has been connected to the processor 4 and the predetermined device is selected in the item ‘peripheral device’ in the column ‘Movie Encode’.

FIG. 9 shows an example of a different setting screen among those of the processor 4, which is displayed as transferred from the setting screen shown in FIG. 8 in response to an operation performed on the keyboard 5 and the like. Now, items that can be set on the setting screen and functions related to the items will be described. The setting screen of the processor 4 shown in FIG. 9 is generated by the graphic circuit 106S (106H) of the image processing unit 72.

Each item of the column ‘Decode’ can be set for display of a still image and a moving image.

In the item ‘Device’ in the column ‘Decode’, a peripheral device, which is recording an image desired by a user to display, can be selected. When ‘TYPE 1’ is selected in the item ‘Device’, the CPU 131 of the main controlling unit 75 reads in an image recorded in the optical recording device 208E1 or 208E2 among the peripheral devices which are connected to the processor 4. When ‘TYPE 2’ is selected in the item ‘Device’, the CPU 131 of the main controlling unit 75 reads in an image recorded in the filing device 204E1 or 204E2 among the peripheral devices which are connected to the processor 4. When ‘TYPE 3’ is selected in the item ‘Device’, the CPU 131 of the main controlling unit 75 reads in an image recorded in the optical recording device 208D1 or 208D2 among the peripheral devices which are connected to the processor 4. When ‘TYPE 4’ is selected in the item ‘Device’, the CPU 131 of the main controlling unit 75 reads in an image recorded in the filing device 204D1 or 204D2 among the peripheral devices which are connected to the processor 4. When ‘TYPE 5’ is selected in the item ‘Device’, the CPU 131 of the main controlling unit 75 reads in an image recorded in the USB (Registered Trademark) memory among the peripheral devices which are connected to the processor 4. When ‘TYPE 6’ is selected in the item ‘Device’, the CPU 131 of the main controlling unit 75 reads in an image recorded in the PC card 167 among the peripheral devices which are connected to the processor 4. When ‘TYPE 7’ is selected in the item ‘Device’, the CPU 131 of the main controlling unit 75 reads in an image recorded in the memory card 168 among the peripheral devices which are connected to the processor 4.

In the item ‘Decode Type’ in the column ‘Decode’, the type of the endoscope composite image to be displayed can be selected and set either from the SDTV or the HDTV.

In the item ‘thumbnail’ in the column ‘Decode’, whether multi image generation is to be performed by using a thumbnail image file or not can be set. When ‘USE’ is selected in the item ‘thumbnail’, the thumbnail/multi-image generating circuit 250 processes to generate a multi image from the thumbnail image file to be inputted. When ‘NO’ is selected in the item ‘thumbnail’, the thumbnail/multi-image generating circuit 250 processes to generate the thumbnail image based on the image to be outputted and also generates a multi image for displaying the thumbnail image.

In the item ‘Mult Num.’ in the column ‘Decode’, the number of images to be displayed in the multi image display can be set between one and 32. The CPU 131 of the main controlling unit 75 controls the thumbnail/multi-image generating circuit 250 of the image decompressing unit 74 so that images are to be displayed by the number set in the ‘Mult Num’. When the item ‘thumbnail’ in the column ‘Decode’ is set to use a thumbnail file, the item ‘Mult Num’ may be disabled and shaded on the display.

In the item ‘Decode Level’ in the column ‘Movie Decode’, the maximum pit rate applied by the moving image decoding circuit 253 of the image decompressing unit 74 in decoding a moving image can be set. The maximum bit rate can be selected from three levels of ‘High’ directing a high image quality, a high bit rate and a big image size, ‘Normal’ directing an image quality lower, a bit rate lower, and an image size smaller than those set in ‘High’, and ‘Low’ directing an image quality still lower, a bit rate still lower, and an image size still smaller than those set in ‘Normal’. In the item ‘Decode level’ in the column ‘Decode’ may become automatically in a predetermined setting according to what is set in the item ‘Encode Type’ in the column ‘Movie Encode’.

In the item ‘SIZE’ in the column ‘Movie Decode’, the display size (aspect ratio) of the moving image can be selected and set from factors 1, 2/3, and 1/2. The CPU 131 of the main controlling unit 75 causes the size change circuit 255 of the image decompressing unit 74 to perform image compression according to each of the factors. When a factor of 1 is selected from the factors, the CPU 131 of the main controlling unit 75 outputs the inputted moving image as it is without causing the size change circuit 255 to perform the compression.

In the item ‘PinP’ in the column ‘Movie Decode’, whether the moving image is to be displayed by PinP or not can be set. FIG. 10 shows an example of the moving image displayed by PinP.

In the item ‘Position’ in the column ‘Movie Decode’, the display position of the moving image that is displayed by PinP can be selected from the upper left, the lower left, the upper right, and the lower right.

Now, functions which can be allocated to any of the abovementioned items of ‘Scope Switch’, ‘Foot Switch’, ‘Keyboard’ and ‘Front Panel’ and operations performed by each unit of the processor 4 to implement the functions will be described. The operations performed by the keys and switches to which the functions are allocated are detected by the CPU 131 via the SIO 142 or the PIO 143 and the system bus 131a.

The ‘Freeze’, one of the functions which can be selected, can issue freeze direction for outputting a freeze image. When a key or a switch to which the freeze function is allocated is operated, the CPU 131 controls the freeze circuit 96 and the memory 97 via the BUF 139 to cause the circuit to output the freeze image. In the present embodiment, the key or switch to which the abovementioned freeze function is allocated is referred to as the freeze switch.

The ‘SFreeze’, one of the functions which can be selected, can issue S freeze direction for outputting an S freeze image. Specifically, ‘SFreeze’ is a function for issuing S freeze direction to output an S freeze image on the left of the screen when the display size is 16:9 that is available for displaying (at least) two images in the endoscope composite image. When a key or a switch to which the S freeze function as mentioned above is allocated is operated, the CPU 131 controls the composition/masking processing circuit 108H via the BUF 139 to store an S freeze image in the memory 112H and also generate and output the endoscope composite image in which the S freeze image is displayed on the left of the screen and an image other than the S freeze image (etc., a freeze image or a moving image) is displayed on the right of the screen. In the present embodiment, a key or a switch to which the abovementioned S freeze function is allocated is referred to as the S freeze switch.

The ‘Release1’, which is a function that can be selected, is a function for issuing a release direction for recording a still image in a peripheral device (an appliance to record the image) and the like. When a key or a switch to which the release function as mentioned above is allocated is operated, the CPU 131 controls the graphic circuit 106S or (and) 106H and outputs the values which are the value of the SCV 309 and the value of the D.F 311 on the screen shown in FIG. 7 incremented by one. When the key or the switch to which the release function is allocated is operated, the CPU 131 records an image to be outputted in the SDTV system in the peripheral device or the like that is set in ‘peripheral device’, which is a sub-item of the item ‘Relase1’ in the column ‘SDTV’, and also records an image to be outputted in the HDTV system in the peripheral device or the like that is set in ‘peripheral device’, which is a sub-item of the item ‘Relase1’ in the column ‘HDTV’.

In the present embodiment, the abovementioned function of ‘Release1’ can allocate the function to up to four keys or switches as ‘Release2’, ‘Release3’ and ‘Release4’.

When any of the keys or switches to which release functions from ‘Release 1’ to ‘Release4’ are allocated respectively, the CPU 131 controls to record an image to be outputted in an appliance to record the image. Now, the control taken by the CPU 131 will be detailed. The functions from ‘Release1’ to ‘Release4’ are the same. Thus, only ‘Release1’ will be described below.

When at least one of the filing devices and photographing devices shown in FIG. 3A, FIG. 3B and FIG. 3C, for example, is selected as an appliance to record the image in ‘Release1’ on the setting screen shown in FIG. 8, the CPU 131 controls to record an image to be outputted in the abovementioned at least one of the devices via the SIO 142 or the PIO 143.

When at least one of the filing devices, photographing devices, and optical recording devices shown in FIG. 3D, for example, is selected as an appliance to record the image in ‘Release1’ on the setting screen shown in FIG. 8, the CPU 131 controls to record an image to be outputted, which is read in from the memory 126 and then outputted from the controller 232 of the image compressing unit 73, into the abovementioned at least one of the devices via the controller 164 of the expansion controlling unit 77A.

When either the PC card 167 or the memory card 168 shown in FIG. 2D, for example, is selected, as an appliance to record the image in ‘Release1’ on the setting screen shown in FIG. 8, the CPU 131 controls to record an image to be outputted, which is read in from the memory 126 and then outputted from the controller 232 of the image compressing unit 73, into the abovementioned at least one of the devices via the controller 165 of the expansion controlling unit 77A.

When at least one of the filing devices, photographing devices, and optical recording devices shown in FIG. 3E, for example, is selected as an appliance to record the image in ‘Release1’ on the setting screen shown in FIG. 8, and it is set to record an image compressed by a high compression rate, the CPU 131 controls to record an image to be outputted, which is read in from the memory 126 and then outputted from the controller 232 of the image compressing unit 73, into the abovementioned at least one of the devices via the HUB 162 and the signal line 162a, and also controls the buffer 166 to record the image to be outputted as a backup image. When at least one of the filing devices, photographing devices, and optical recording devices shown in FIG. 3E, for example, is selected as an appliance to record the image in ‘Release1’ on the setting screen shown in FIG. 8, and it is set to record an image compressed by a high compression rate, the CPU 131 controls the buffer 166 to record an image to be outputted that is read in from the memory 126 and then outputted from the controller 232 of the image compressing unit 73. Then, when a key with a function of reporting the end of examination or the like is operated and the end of examination is reported, some or all of the images to be outputted which are recorded in the buffer 166 are recorded in at least one of the filing devices, photographing devices and optical recording devices shown in FIG. 3E.

‘Iris’, one of the functions which can be selected, is a function for selecting or switching the photometry (light-controlling) system from among Peak, Average, and Automatic. When a key or a switch to which such a function as the photometry switching function is allocated is operated, the CPU 131 generates the light-controlling signal based on the direction according to the operation and outputs the light-controlling signal to the light equipment 3 via the signal line 59a and the like. ‘Enhance’, one of the functions which can be selected, is a function for selecting or switching highlighting of an image from or among the structure highlighting and edge highlighting, for example. When a key or a switch to which such a function as the highlighting function is allocated is operated, the CPU 131 controls the graphic circuit 106S or (and) 106H and outputs the display with the contents of the structure highlighting/edge highlighting 323A or (and) 323B on the screen shown in FIG. 7 changed. When the key or the switch to which the highlighting switching function is allocated is operated, the CPU 131 controls the zoom-up/highlight circuit 99H or (and) 99S via the BUF 139 and outputs a highlighted image to be outputted.

‘Contrast’, one of the functions which can be selected, is a function for selecting or switching the contrast of an image from or among ‘Low’ (low contrast), ‘Normal (medium contrast)’, ‘High’ (high contrast) and non-correction. When a key or a switch to which such a function as the contrast switching function is allocated is operated, the CPU 131 controls the graphic circuit 106S or (and) 106H and outputs the display with the contents of the contrast 320 A or (and) 320B on the screen shown in FIG. 7 changed. When the key or the switch to which the contrast switching function is allocated is operated, the CPU 131 controls the pre-stage image processing circuit 95 via the BUF 139 to perform γ conversion based on a direction according to the operation.

‘Img. Size’, one of the functions which can be selected, is a function for switching the image size of the image to be outputted. When a key or a switch to which such a function as the image size switching function is allocated is operated, the CPU 131 controls the zoom-up/highlight circuit 99H or (and) 99S via the BUF 139 and outputs (the enlarged image) by changing the image size of the image to be outputted. When the key or the switch to which the image size switching function is allocated is operated, the CPU 131 controls composition/masking processing circuit 108H or (and) 108S via the BUF 139 and combines and outputs the image with the changed image size and the masked image signal

‘VTR’, one of the functions which can be selected, is a function for toggling recording a moving image in a VTR and a halt of recording the moving image in the peripheral device connected to the processor 4. When a key or a switch to which such a function as the VTR recording function is allocated is operated, the CPU 131 controls the graphic circuit 106S or (and) 106H and outputs the display with the contents of the VTR 312 shown in FIG. 7 changed (‘VTR’ is displayed while a moving image is being recorded, and ‘VTR’ is hidden while the recording is halted). Each time when the key or the switch to which the VTR recording function is allocated is operated, the CPU 131 alternately outputs a direction for one or more of the VTRs 203A, 203B1, 203B2, 203C1 and 203C2 among peripheral devices which are connected to the processor 4 to record a moving image and a direction for them to halt the recording of the moving image. When the key or switch to which the VTR recording function is allocated is operated while a moving image is played on a VTR, the CPU 131 stops playing of the moving image. Each time when the key or the switch to which the VTR recording function is allocated is operated, the CPU 131 alternately outputs a direction to record a moving image other than the abovementioned moving image and a direction to halt the recording of the moving image other than the abovementioned moving image. The direction to record a moving image and the direction to halt the recording of the moving image by the VTR recording function as mentioned above may be outputted to the filing devices 204C1 and 204C2 as well as the VTRs. The VTRs shown in FIG. 3A to FIG. 3C may have a switch and the like with the VTR recording function, which are independent of functions allocated by the processor 4.

‘Capture’, one of the functions which can be selected, is a function for capturing a still image at a printer among peripheral devices which are connected to the processor 4. When a key or a switch to which such a function as the capture function is allocated is operated, the CPU 131 controls the graphic circuit 106S or (and) 106H and outputs the display with the contents of the CVP 310 shown in FIG. 7 (count and the number of pages in the memory, etc.) changed. When the key or the switch to which the capture function is allocated is operated, the CPU 131 outputs a direction to capture an image to be outputted and the image to be outputted to a printer among peripheral devices which are connected to the processor 4.

Now, control taken by the CPU 131 to cause an objective appliance to capture an image to be outputted when either the key or the switch to which the capture function by ‘Capture’ is allocated is operated will be detailed.

When at least one of the printers shown in FIG. 3A, FIG. 3B and FIG. 3C captures an image to be outputted, the CPU 131 controls the printer to capture the image to be outputted via the SIO 142 or the PIO 143.

When at least one of the printers shown in FIG. 3D is selected, the CPU 131 controls to cause the printer to capture an image to be outputted that is read in from the memory 126 and then outputted from the controller 232 of the image compressing unit 73 via the controller 164 and the like of the expansion controlling unit 77A.

When at least one of the printers shown in FIG. 3E is selected and it is set to capture an image compressed by a high compression rate, the CPU 131 controls to cause the printer to capture an image to be outputted that is read in from the memory 126 and outputted from the controller 232 of the image compressing unit 73 via the HUB 162 and the signal line 162a and also to cause the buffer 166 to record the image to be outputted. When at least one of the printers shown in FIG. 3E is selected and it is set to record an image compressed by a low compression rate, the CPU 131 controls to cause the buffer 166 to record the image to be outputted that is read in from the memory 126 and outputted from the controller 232 of the image compressing unit 73. When the key with the function of reporting the end of examination is operated and the end of examination is reported, some or all of the images to be outputted which are recorded in the buffer 166 are captured by at least one of the printers shown in FIG. 3E.

The printer may be selected on the setting screen shown in FIG. 8.

‘Print, one of the functions which can be selected, is a function for causing a printer among peripheral devices which are connected with the processor 4 to print and output a still image. When a key or a switch to which such the function as the print function is allocated is operated, the CPU 131 outputs a direction to a printer among peripheral devices which are connected with the processor 4 to print an image to be outputted.

Now, control taken by the CPU 131 to cause an objective appliance to print an image to be outputted when a key or a switch to which a print function by ‘Print’ is allocated is operated will be detailed.

When one of the printers shown in FIG. 3A, FIG. 3B and FIG. 3C prints an image to be outputted, the CPU 131 controls to print a still image captured in the printer via the SIO 142 or the PIO 143.

When at least one of the printers shown in FIG. 3D is selected, the CPU 131 controls to print a still image captured in the printer via the controller 164 of the expansion controlling unit 77A.

When at least one of the printers shown in FIG. 3E is selected, the CPU 131 controls to print a still image captured in the printer via the HUB 162, the signal line 162a and the like.

‘Stop W.’, one of the functions which can be selected, is a function for switching the display state and the operation state of the stop watch in the time information 308 on the screen shown in FIG. 7. When a key or a switch to which such the function as the stop watch function is allocated is operated, the CPU 131 controls the graphic 106S or (and) 106H based on a time shown in the RTC 134, and switches the display state of the stop watch in the time information 308 on the screen shown in FIG. 7. In the present embodiment, the display state of the stop watch is serially switched among displaying a stop watch and starting an operation; stopping the stop watch; and hiding the stop watch each time when a key to which the stop watch function is allocated is operated.

‘UPD’, one of the functions which can be selected, is a function for toggling displaying and hiding an endoscope form image that is generated and outputted at the graphic circuit 169 of the expansion controlling unit 77B. When a key or a switch to which such a function as the UPD image switching function is allocated is operated, the CPU 131 controls whether or not to combine the endoscope form image that is outputted from the graphic circuit 169 of the expansion controlling unit 77B at the composition/masking processing circuit 108H or (and) 108S and output the image based on the direction according to the operation. (For the processing of the control, see the parts describing about the processing from step DDDFLW4 to step DDDFLW7 shown in FIG. 2H).

‘ZScale’, one of the functions which can be selected, is a function for toggling displaying and hiding zoom control information that is outputted from the expansion controlling unit 77B. When a key or a switch to which such a function as the ZScale image switching function is allocated is operated, the CPU 131 controls whether or not to make the zoom control information into an image at the graphic circuits 106S and 106H and mask and output the zoom control information at the composition/masking processing circuit 108H and the composition/masking processing circuit 108S based on the direction according to the operation. (For the processing of the control, see the parts describing about the processing from step DDDFLW4 to step DDDFLW7 shown in FIG. 2H).

‘Zoom’, one of the functions which can be selected, is a function for switching the factor of electronic zoom-up performed on an image to be outputted. When a key or a switch to which such a function as the electronic zoom-up function is allocated is operated, the CPU 131 controls the zoom-up/highlight circuit 99H or (and) 99S via the BUF 139 to perform electronic zoom-up by the factor based on the direction according to the operation.

‘IHb’, one of the functions which can be selected, is a function for switching a degree of color highlight according to the hemoglobin index. When a key or a switch to which such a function as the hemoglobin index color highlight function is allocated is operated, the CPU 131 controls the graphic circuit 106S or (and) 106H and outputs the display with the contents of the color highlight 321A or (and) 321B on the screen shown in FIG. 7 changed. When a key or a switch to which the hemoglobin index color highlight function is allocated is operated, the CPU 131 controls the post-image processing circuit 98 via the BUF 139 about the degree of IHb color highlight processing, which is the color highlight processing according to the hemoglobin index.

‘PUMP’, one of the functions which can be selected, is a function for toggling switching ON and OFF the forward-water-feeding pump (not shown) to feed water. When a key or a switch to which such a function as the forward-water-feeding pump function is allocated is operated, the CPU 131 controls the forward-water-feeding pump (not shown) to start or stop the forward-water-feeding. When a key or a switch to which the forward-water-feeding function is allocated is operated, the CPU 131 controls the graphic circuit 106S or (and) 106H and outputs the display of the PUMP 313 on the screen shown in FIG. 7 with the contents changed.

‘Exam End’, one of the functions which can be selected, is a function for reporting the end of examination to a peripheral device and the like that is connected to the processor 4. When a key or a switch to which such a function as the end of examination reporting function is allocated is operated, the CPU 131 controls the graphic circuit 106S or (and) 106H and clears a part of information included in the group of observe information 300 which is displayed on the screen shown in FIG. 7 (and displays the item name in the place). When a key or a switch to which the end of examination reporting function is allocated is operated, the CPU 131 outputs a signal directing the end of examination to each unit of the processor 4.

‘M-REC’, one of the functions which can be selected, is a function for toggling recording a moving image and a halt of recording a moving image in an optical recording device and a filing device among the peripheral devices connected to the processor 4. When a key or a switch to which such a function as the moving image recording function is allocated is operated, the CPU 131 controls the graphic circuit 106S or (and) 106H and outputs the display state with the contents of the VTR 312 shown in FIG. 7 changed (‘VTR’ is displayed while a moving image is being recorded, and ‘VTR’ is hidden while the recording is halted). Each time when the key or the switch to which the moving image recording function is allocated is operated, the CPU 131 alternately outputs a direction for one or more of the filing devices 204D1, 204D2, 204E1, and 204E2 and the optical recording devices 208D1, 208D2, 208E1 and 208E2 among peripheral devices which are connected to the processor 4 to record a moving image and a direction for them to halt the recording of the moving image. The filing devices and (or) the optical recording devices shown in FIG. 3D and FIG. 3E may have a switch and the like with the moving image recording function which are independent of functions allocated by the processor 4.

‘Special light’, one of the functions which can be selected, is a function for toggling to select and switch filters arranged on an optical path of the lamp 51 among the special light filters 53A, 53B and 53C of the light equipment 3. When a key or a switch to which such a function as the special light filter switching function is allocated is operated, the CPU 131 controls the graphic circuit 106S or (and) 106H and outputs the screen shown in FIG. 7 with the display state of the light source filter type 325A or (and) 325B changed. When a key or a switch to which such a function as the special light filter switching function is allocated is operated, the CPU 131 changes filters arranged on a light path of the lamp 51 of the light equipment 3 by performing control based on the direction according to the operation via the signal line 59a and the like. When a key or a switch to which the special light filter switching function is allocated is operated, the CPU 131 controls each of the pre-stage image processing circuit 95, the post-stage image processing circuit 98, the zoom-up/highlight circuit 99H and the zoom-up/highlight circuit 99S and causes those circuits to perform the image processing according to the type of the filter arranged on the light path of the lamp 51.

‘P-VTR’, one of the functions which can be selected, is a function for toggling playing a moving image recorded in a VTR among peripheral devices connected to the processor 4 and a halt of playing the moving image. When a key or a switch to which such a function as the VTR playing function is allocated is operated, the CPU 131 controls the graphic circuit 106S or (and) 106H and outputs the screen shown in FIG. 7 with the display state of the VTR 312 changed (‘VTR’ is displayed while a moving image is being played, and ‘VTR’ is hidden while the playing is halted). Each time when the key or the switch to which the VTR playing function is allocated is operated, the CPU 131 alternately outputs a direction for one of the VTRs 203A, 203B1, 203B2, 203C1 and 203C2 among peripheral devices which are connected to the processor 4 to play a moving image and a direction for them to halt the playing of the moving image. When the key or switch to which the VTR playing function is allocated is operated while a moving image is recorded on a VTR, or while a moving image is fast forwarded, or while a moving image is fast rewinded, the CPU 131 stops the processing (recording of a moving image, fast forwarding or fast rewinding of a moving image), and alternately outputs a direction to play the moving image and a direction to halt the playing of the moving image each time when the key or the switch is operated. The direction to play the moving image by using the VTR playing function and the direction to halt the playing of the moving image may also be outputted to the filing devices 204C1 and 204C2 other than the abovementioned VTRs.

‘M-PLY’, one of the functions which can be selected, is a function for toggling playing a moving image in the optical recording device and the filing device among peripheral devices which are connected to the processor 4 and a halt of playing the moving image. When a key or a switch to which such a function as the moving image playing function is allocated is operated, the CPU 131 controls the graphic circuit 106S or (and) 106H and outputs the screen with the display state of the VTR 312 shown in FIG. 7 changed (‘VTR’ is displayed while a moving image is being played, and ‘VTR’ is hidden while the playing is halted). Each time when the key or the switch to which the moving image playing function is allocated is operated, the CPU 131 alternately outputs a direction for one of the filing devices 204D1, 204D2, 204E1, and 204E2 and the optical recording device 208D1, 208D2, 208E1, and 208E2 among peripheral devices which are connected to the processor 4 to play a moving image and a direction to halt the playing of the moving image. The filing devices and (or) the optical recording device shown in FIG. 3D and FIG. 3E may have a switch and the like with the moving image playing function, which are independent of functions allocated by the processor 4.

‘NET’, one of the functions which can be selected, is a function for toggling whether to display or hide (the image based on) the network related information that is outputted from the expansion controlling unit 77A. When a key or a switch to which such a function as the network related information image switching function is allocated is operated, the CPU 131 controls whether or not to combine (the image based on) the network related information outputted from the expansion controlling unit 77A at the composition/masking processing circuit 108H or (and) 108S and to output it based on the direction according to the operation. (For the processing of the control, see the parts describing about the processing from step DDDFLW4 to step DDDFLW7 shown in FIG. 2H).

‘TELE’, one of the functions which can be selected, is a function for moving the objective optical system 22A (22B) of the endoscope 2A (2B) toward the zooming-up direction (TELE). While a key or a switch to which such a function as the TELE function is allocated is being operated, the CPU 131 drives the actuator 23A (23B) of the endoscope 2A (and 2B) via the driving circuit 186 of the expansion controlling unit 77B and moves the objective optical system 22A (22B) in the zooming-up direction (TELE), which is the axial direction and also the direction toward the distal end portion of the insertion portion 21A (21B). When a key or a switch to which such a function as the TELE function is operated, the CPU 131 controls the graphic circuit 106S or (and) 106H, and outputs the display with the contents of the zoom control information changed to the contents appropriate for the zooming up (TELE).

‘WIDE’, one of the functions that can be selected, is a function for moving the objective optical system 22A (22B) of the endoscope 2A (2B) toward the wide angle (WIDE) direction. While a key or a switch to which such a function as the wide function is allocated is operated, the CPU 131 drives the actuator 23A (23B) of the endoscope 2A (and 2B) via the driving circuit 186 of the expansion controlling unit 77B and moves the objective optical system 22A (22B) in the wide angle direction (WIDE), which is the axial direction and also the direction toward the distal end portion of the insertion portion 21A (21B). When a key or a switch to which such a function as the WIDE function is operated, the CPU 131 controls the graphic circuit 106S or (and) 106H, and outputs the display with the contents of the zoom control information changed to the contents appropriate for the wide angle (WIDE).

‘OFF’, one of the functions which can be selected is the setting for preventing any of the functions mentioned above from being allocated. Specifically, when a key or a switch to which ‘OFF’ is set is operated, the processor 4 performs no processing.

The CPU 131 may be adopted to select only some of the functions according to the detected result and the like of the connection status of the expansion controlling units 77A and 77B, for example, among the abovementioned functions. Specifically, the CPU 131 may be adopted to disable selection or display of the functions related to those unconnected (or those undetected) among the expansion controlling units 77A and 77B.

Now, processing and the like performed by each unit of the processor 4 when a key or a switch with the moving image recording function is operated and a moving image is recorded will be described.

When a key or a switch with the moving image recording function is operated, the CPU 131 of the main controlling unit 75 sets the selector 124, the selector 234, the size change circuit 235, the YUV converting circuit 236 and the moving image encoding circuit 237 based on the selection done in each item in the column ‘Movie Encode’ on the setting screen shown in FIG. 8. Then, the moving image outputted from the moving image encoding circuit 237 is outputted to a peripheral device via the bus bridge 163 as in the manner to be described below.

The moving image outputted from the moving image encoding circuit 237 is subjected to format conversion by the CPU 151 of the expansion controlling unit 77A, encryption by the encrypting circuit 170, and outputted to the filing device 204E1 (and 204E2) and the optical recording device 208E1 (and 208E2) via the signal line 162a together with endoscope related information, security information and the like added. The protocol for outputting the moving image via the signal line 162a may be any of the TCP/IP, the FTP, the HTTP, the XML, the HL7, the SGML, the JAVA (Registered Trademark), the COM, the DCOM, the CORBA, the DBMS, and the RDBMS.

The moving image outputted from the moving image encoding circuit 237 is subjected to format conversion by the CPU 151 of the expansion controlling unit 77A, encryption by the encrypting circuit 170, and outputted to the filing device 204D1 (and 204D2), the optical recording device 208D1 (and 208D2), and a USB (Registered Trademark) memory (not shown) via the controller 164 together with endoscope related information, security information and the like added. The Class Driver of the USB (Registered Trademark) may be the HUB Class Driver, the Human Interface Devices Class Driver, the Communication Device Class Driver, the Audio Class DriVer, the Mass Storage Class Driver, the Still Image Capture Device Class Driver, the Printer Class Driver and the like, or may correspond to the USB (Registered Trademark) On-The-Go standard. When the moving image encoding circuit 237 performs the format conversion on a moving image, the moving image may be directly outputted to the controller 164 without passing through the CPU 151.

The moving image outputted from the moving image encoding circuit 237 is subjected to format conversion by the CPU 151 of the expansion controlling unit 77A, encryption by the encrypting circuit 170, and outputted to the PC card 167 and (or) the memory card 168 via the card controller 165 via the card controller 165 together with the endoscope related information, the security information and the like added. When the moving image encoding circuit 237 performs the format conversion on a moving image, the moving image may be directly outputted to the card controller 165 without passing through the CPU 151.

The moving image outputted from the moving image encoding circuit 237 may be outputted to the peripheral devices in such a manner to be first outputted to any of the PC card 167 and (or) the memory card 168 or the buffer 166, and finally to a peripheral device which is connected to the signal line 162a and the controller 164, and the state of recording the moving image may be stored in the backup RAM 137 (or 155). Accordingly, even if the processor 4 is switched off and then switched on while a moving image is being recorded to a peripheral device that is connected to the signal line 162a and the controller 164, the CPU 131 (or the CPU 151) can automatically output the moving image which has been recorded to any of the PC card 167, the memory card 168 or the buffer 166 to the peripheral device that is connected to the signal line 162a and the controller 164 by reading in the information directing the recording state of the peripheral device that has been store din the backup RAM 137 (or 155).

The directory structure used in recording an image in the filing devices, the optical devices, the PC cards 167, the memory cards 168 and the USB (Registered Trademark) memory shown in FIG. 3A to FIG. 3E may be a common structure such as each file is stored in the directory associated with the patient ID as shown in FIG. 11A, for example. The directory structure shown in FIG. 11A is merely an example in the case of a still image, though, a structure like that may be used in the case of the moving image (with an extensions shown as ‘.mpg’, for example). A still image and a moving image under the same patient ID are not limited to be stored in different directories in the memory, and the still image and the moving image under the same patient ID may be stored in the same directory together. At least one of authentication information including a guard key, a password, a user's finger print, iris, authentication, blood-vessel arrangements in the eye's retina, handwriting, a voice pattern, a face shape, a signature, vein arrangements on the back of the hand may be added to each directory and (or) file in the directory structure shown in FIG. 11A as the security information. That allows only a predetermined user to access a file (and a directory). As a result, each type of information related to a patient can be protected more firmly. Access to the directories and (or) the files in the directory structure shown in FIG. 11A may be limited (such as to be overwritten) by any of the guard key, the password or the authentication information.

The directory name and the file name in the directory structure shown in FIG. 11A are not limited to be generated based on the patient ID, and may be automatically generated based on any of the elements of the group of observe information 300, may be open for a user to input a desired name, may be what written with limited characters, or may be such that an appropriate name is automatically generated according to the limited characters and the group of observe information 300. The directory name and the file name in the directory structure shown in FIG. 11A may be such that different names are automatically generated according to whether the image to be recorded is a freeze image or an S freeze image.

The image file of thumbnail images and the image file of images originated the thumbnail images among files in the directory structure shown in FIG. 11A may be different as shown in FIG. 11B, or may be combined in an image file as shown in FIG. 11C. ‘SOI’ is the information directing the beginning of the file data and ‘EOI’ is the information directing the end of the file data in FIG. 11B and FIG. 11C.

At least a piece of information and the like among pieces of information listed from the item a) to the item z) below, for example, are added to images (a moving image and a still image) recorded in the peripheral device and the like.

a) A group of observe information 300 and setting information related to the group of observe information 300 shown in FIG. 7.

b) A group of image related information 301A (302A) and setting information related to the group of image related information 301A (302A).

c) Connection information of a peripheral device (the number of recorded sheets, the recording state, the presence of connection, the power source state, the communication state, the division mode or the number of printed sheets for a printer, an operation state of a VTR (play, record, stop)).

d) Information related to the endoscope image 301 (302) other than the group of image related information 301A (302A) (setting or the like of an IHb pseudo color display region, the image size (any of Medium, Semi-Full or Full), monochrome).

e) Functions allocated to the operation switching section 28A (28B) of the endoscope 2A (2B), the keyboard 5, and the front panel 76 (Input setting or the like for Caps Lock, Insert, and characters from the keyboard 5).

f) A display state of the arrow pointer 301a (302a).

g) An operation state of the stop watch of the time information 308 (during operation or being halted).

h) Information on whether the time information 308 is omitted in the display or not.

i) All messages displayed in the endoscope composite image.

j) A display size (screen aspect ratio) of the endoscope composite image.

k) The number of the thumbnail images 326 of the group of the thumbnail images 326A.

l) A display state of each piece of information on the endoscope composite image (display or delete).

m) Information stored in the memory 30A (30B) of the endoscope 2A (2B).

n) A serial number of the processor 4.

o) The number of times the processor 4 is switched ON.

p) The date and time when an image is recorded.

q) The type of the endoscope 2A (2B).

r) Setting state of the photometry (light-controlling) (peak, average, or automatic).

s) An Mac address and an IP address of the Ethernet (Registered Trademark).

t) A data size of an image.

u) A reduction rate of an image.

v) A color space of an image (s RGB and the like).

w) Identification of an image.

x) Setting for each setting screen (shown in FIG. 8, FIG. 9 and the like).

y) A header file, a marker and the like of the format.

z) A serial number and the product name of an appliance that is to record an image.

The image size (any of Medium, Semi-Full or Full) in the item d) can be changed in response to an operation performed on a key or a switch to which an image size switching function is allocated.

Now, the processing performed by each unit of the processor 4 to play a moving image in response to an operation performed on a key or a switch having the moving image playing function will be described.

When the key, the switch or the like having the moving image playing function is operated, the CPU 131 of the main controlling unit 75 controls to read in the directory name and the file name stored in the peripheral device and the like and display them in a display form corresponding to the directory structure shown in FIG. 11A as shown in FIG. 12, for example. The display shown in FIG. 12 is an example in the case of a still image, and the similar display may be used in the case of a moving image (such as denoted by the extension ‘.mpg’).

Then, when a predetermined key or a predetermined switch of the operating device (for example, a predetermined switch of the keyboard 5, the HID 209D1 or the like, for example) is pressed and a directory name is selected, and then a confirmation key (for example, ENTER key on the keyboard 5 or the like) is pressed and the objective file is confirmed, the CPU 131 of the main controlling unit 75 displays a message directing that it is under preparation for display (for example, a message like ‘Please Wait’), and then controls for playing a moving image. When a mouse is connected as the HID 209D1 or 209D2, the CPU 131 may be adopted to take a double-click of the mouse as the input similar to the pressing of the confirmation key.

In controlling to play the moving image, the CPU 151 outputs the moving image that will make an objective file via the bus bridge 163, the moving image decoding circuit 253, the RGB conversion circuit 253, and the sizes change circuit 255.

In controlling to play the moving image, the size change circuit 255 outputs the inputted moving image by changing the size according to the size set in the item ‘SIZE’ in the column ‘Decode’ on the setting screen shown in FIG. 9.

In controlling to play the moving image, when the moving image is outputted in the HDTV system according to the setting in the item ‘Decode Type’ in the column ‘Decode’ on the setting screen shown in FIG. 9 under the control of the CPU 151, the selector 256 selects the clock signal of 74 MHz, and when the moving image is outputted in the SDTV system, the selector 256 selects the clock signal of 13.5 MHz. The CPU 151 (or the CPU 131) may be adopted to only output the moving image in the system set in the item ‘Decode Type’ in the column ‘Decode’ on the setting screen shown in FIG. 9. Specifically, the CPU 151 (or the CPU 131) may be adopted to display only the moving image that is outputted via the composition/masking processing circuit 108S when the ‘SDTV’ is selected in the item ‘Decode Type’ in the column ‘Decode’, and display only the moving image that is outputted via the composition/masking processing circuit 108H when the ‘HDTV’ is selected in the item ‘Decode Type’ in the column ‘Decode’.

The trick play (fast forward, fast rewind, halt, stop) may be performed on the moving image that is outputted from the processor 4 in response to respective operations performed on predetermined keys or predetermined switches of the operating device.

Control and processing performed by the CPU 131 of the main controlling unit 75 when a still image recorded on a peripheral device or the like is displayed will be described with reference to the flowchart shown in FIG. 13.

First, the CPU 131 of the main controlling unit 75 detects whether a recorded image display directing key provided on an operating device, for example, has been inputted or not via either the SIO 142 or the PIO 143 (step CFLW1 shown in FIG. 13). Detection on whether the recorded image display directing key of the HIDs 209D1 and 209D2 in each operating device has been inputted or not is not limited to be performed by the CPU 131, and it may be performed by the CPU 151 of the expansion controlling unit 77A and the detection may be outputted to the CPU 131 via the SIO 159, the SIO 142 and the like.

When the CPU 131 detects that the recorded image display directing key has been inputted, it controls to generate and output a message (e.g., a message like ‘Please Wait’) or an image (an image such as a black screen and a color bar) directing that it is under preparation for displaying a still image in any of the graphic circuit 106H, the graphic circuit 106S, and the graphic circuit 169 (step CFLW2 shown in FIG. 13). The message or the image directing that it is in preparation for displaying will be referred to as Wait Screen in the specification (and the drawings) below. The processing performed when the Wait Screen is displayed is similar to that performed in step CFLW2 shown in FIG. 13 unless otherwise particularly described.

Then, the CPU 131 controls to read in the directory name and the image file name stored in the peripheral device and the like and display them as shown in FIG. 12, for example (step CFLW3 shown in FIG. 13). The peripheral device referenced by the CPU 131 in the processing at step CFLW3 shown in FIG. 13 is the appliance set in the item ‘Device’ in the column ‘Decode’ on the screen shown in FIG. 9.

The CPU 131 is not limited to use the display system shown in FIG. 12 in displaying the directory name and the image file name stored in the peripheral device (the appliance set in the item ‘Device’ in the column ‘Decode’ on the setting screen shown in FIG. 9) to which the CPU 131 is referring, and it may display only the image and the thumbnail in the type (the SDTV or the HDTV) set in the item ‘Decode Type’ in the column ‘Decode’ on the setting screen shown in FIG. 9 based on information including size information, the identification, the reduction rate and (or) the data size added to the image. The CPU 131 may be adopted to display only the directory name at first when it is to be display the directory name and the image file name stored in the peripheral device or the like it referenced, and display the image file name stored in the directory only when it detects that a directory is selected and a predetermined key (or switch) is inputted (for example, a right-click on a mouse, which is an HID). A predetermined key (e.g., the keyboard 5, or character keys and the like on the HID 209D1 and 109D2) may change the directory name and the image file name selected in response to operation performed on the operating device. The CPU 131 may be adopted to display one or more pages when there are many directories and (or) image files.

When a predetermined key (e.g., an arrow key and the like on the keyboard 5) of the operating device is pressed and a directory is selected, and a confirmation key (e.g., ENTER key and the like on the keyboard 5) is pressed and a directory is confirmed (step CFLW4 shown in FIG. 13), the CPU 131 performs processing for displaying the Wait Screen (step CFLW5 shown in FIG. 13) and also causes to generate and output a multi image in the Wait Screen (step CFLW6 shown in FIG. 13).

Now, the processing performed at step CFLW6 shown in FIG. 13 will be detailed.

The CPU 131 reads in the image files in the directory stored in the peripheral device (the appliance set in the item ‘Device’ in the column ‘Decode’ on the setting screen shown in FIG. 9) to which the CPU 131 is referring, so that the image files in the memory 242 via the bus bridge 163 and the controller 241 are stored. The image files stored in the memory 242 in the processing is not limited to all image files in the directory and may be only the thumbnail image file. When the image files in the directory stored in the peripheral device or the like to which the CPU 131 is referring have been subjected to encryption, the CPU 131 decodes the image file by the encrypting circuit 170 and store it in the memory 242.

Then, the CPU 131 causes the image decompressing unit 74 to serially output the image files stored in the memory 242, while controlling the selectors 243, 245, 246 and 248 so that appropriate decompression/conversion and RGB conversion according to the format and the like of the image files is performed based on the information added to the image files stored in the memory 242. The CPU 131 also controls the selectors 249 and 251 so that the image files outputted from the memory 242 is to be outputted via the thumbnail/multi-image generating circuit 250.

When ‘USE’ is selected in the item ‘thumbnail’ in the column ‘Decode’ on the setting screen shown in FIG. 9, the thumbnail/multi-image generating circuit 250 performs processing for generating a multi image according to the image size based on the image size of the thumbnail image file. Specifically, when the thumbnail image file in the SDTV system in the size of 180×120 is inputted, the thumbnail/multi-image generating circuit 250 generates a multi image with 16 images arranged on a screen and outputs the multi image.

When ‘NO’ is selected in the item ‘thumbnail’ in the column ‘Decode’ on the setting screen shown in FIG. 9, the thumbnail/multi-image generating circuit 250 performs processing for generating a multi image from the image file to be inputted. Specifically, the thumbnail/multi-image generating circuit 250 generates thumbnail images by the number set in the ‘Mult Num.’ in the column ‘Decode’ on the setting screen shown in FIG. 9 and generates a multi image with the thumbnail images arranged on a screen and outputs the multi image.

The multi image generated by the thumbnail/multi-image generating circuit 250 is inputted to the synchronous circuit 252 and then serially outputted frame by frame based on the frequency of the clock signal. Specifically, when the multi image generated by the thumbnail/multi-image generating circuit 250 is in the SDTV system, the synchronous circuit 252 outputs the multi image to the composition/masking circuit 108S at timing to synchronize to the clock signal of 3.5 MHz. When the multi image generated by the thumbnail/multi-image generating circuit 250 is in the HDTV system, the synchronous circuit 252 outputs the multi image to the composition/masking circuit 108H at timing to synchronize to the clock signal of 74 MHz.

The CPU 131 may be adopted to control to display only the multi image in the type (the SDTV or the HDTV) set in the item ‘Decode Type’ in the column ‘Decode’ on the setting screen shown in FIG. 9 among the multi-images outputted from the synchronous circuit 252. Specifically, the CPU 131 may be adopted to control to display only a multi image outputted from either the composition/masking processing circuit 108H or the composition/masking processing circuit 108S which matches setting (the SDTV or the HDTV) performed in the item ‘Decode Type’ in the column ‘Decode’ on the setting screen shown in FIG. 9, and display a predetermined image such as a black screen or a blue screen or an error display instead of the other multi image outputted from the circuit which does not match the setting without displaying the other multi image.

According to the processing at step CFLW6 shown in FIG. 13, the multi image is generated and outputted as shown in FIG. 14A.

The heavy-lined frame in the multi image shown in FIG. 14A is a frame for selecting an image that is currently selected among images included in the multi image. The heavy-lined frame can be moved in response to pressing predetermined keys (e.g., an arrow key on the keyboard 5) of the operating device, for example. The selecting frame is generated by the graphic circuit 106H and then combined by the composition/masking processing circuit 108H, and generated by the graphic circuit 106S and combined by the composition/masking processing circuit 108S, and outputted respectively. The selecting frame may be generated by the graphic circuit 169.

As shown in FIG. 14B, the multi images can be switched for each page (a screen of the multi image) in response to pressing of the switch-to-next-page key (e.g., PageUp key on the keyboard 5 or the like) or switch-to-previous-page key (e.g., PageDown key on the keyboard 5 or the like) of the operating device, for example. When the CPU 131 detects that either the switch-to-next-page key or the switch-to-previous-page key is pressed and a page switching direction is issued for the multi images (step CFLW7 shown in FIG. 13), it processes to display the Wait Screen (step CFLW8 shown in FIG. 13) and also generates and outputs the multi image on the specified page while the Wait Screen is being displayed (step CFLW9 shown in FIG. 13). The CPU 131 is not limited to generate the multi images on the specified pages one by one as in the processing at step CFLW9 shown in FIG. 13 and may output the multi image as it is in response to the specification of the generated multi image. The selecting frame directing the currently selected image may be displayed as the multi image at the upper left corner is being selected when pages are changed. If the CPU 131 detects any of the cases where a direction to change the page is issued when there is only one page, where a switch-to-previous-page direction is issued when there is no previous page, or where a switch-to-next-page direction is issued when there is no page next, it may disable pressing of the key on the keyboard 5 or the like and alert with a beep or an error display. The CPU 131 may display the page number (for each of the multi images) on the upper right corner or the like on the multi images.

When the CPU 131 detects that a predetermined key (e.g., Backspace key or ESC key on the keyboard 5 or the like) of the operating device is pressed and a direction to return to the previous screen is issued (step CFLW10 shown in FIG. 13), it controls again to display the Wait Screen according to the processing at step CFLW2 shown in FIG. 13 and then display the directory name and the image file name according to the processing at step CFLW3 shown in FIG. 13.

When the CPU 131 detects that an image in the multi image is selected by the selecting frame, and also detects that the confirmation key (e.g., ENTER key of the keyboard 5 or the like) of the operating device is pressed and the image selection is confirmed (step CFLW11 shown in FIG. 13), the processing to display the Wait Screen (step CFLW12 shown in FIG. 13) is performed and also an original image of the image as the thumbnail image while the Wait Screen is being displayed (step CFLW13 shown in FIG. 13) is outputted.

Now, the processing at step CFLW13 shown in FIG. 13 will be detailed.

The CPU 131 reads in the image file which is the original image of the selected thumbnail image from an appliance (an appliance referenced in the processing at step CFLW6 shown in FIG. 13) set in the item ‘Device’ in the column ‘Decode’ on the setting screen shown in FIG. 9, so that the image file in the memory 242 via the bus bridge 163 and the controller 241 is stored. The CPU 131 may be adopted to perform processing to extract the image file which is the original image of the selected the thumbnail image from the image files stored in the memory 242 when all image files recorded in the appliance set in the item ‘Device’ in the column ‘Decode’ on the setting screen shown in FIG. 9 have been stored in the memory 242 (according to the processing at step CFLW6 shown in FIG. 13).

Then, the CPU 131 causes the original image file stored in the memory 242, while controlling the selectors 243, 245, 246 and 248 so that appropriate decompression/conversion and RGB conversion according to the format and the like of the image files is performed based on the information added to the original image file. The CPU 131 also controls the selectors 249 and 251 so that the original image file outputted without passing through the thumbnail/multi-image generating circuit 250. According to such a processing in the image decompressing unit 74, the compressed original image file is outputted from the selector 251 as the decompressed original image.

The original image outputted from the selector 251 is inputted to the synchronous circuit 252 and then outputted based on the frequency of the clock signal. Specifically, when the original image is in the SDTV system, the synchronous circuit 252 outputs the original image to the composition/masking processing circuit 108S at timing to synchronize to the clock signal of 13.5 MHz. When the original image is in the HDTV system, the synchronous circuit 252 outputs the original image to the composition/masking processing circuit 108H at timing to synchronize to the clock signal of 74 MHz.

The CPU 131 may be adopted to control to display only the original image in the type (the SDTV or the HDTV) set in the item ‘Decode Type’ in the column ‘Decode’ on the setting screen shown in FIG. 9. Specifically, the CPU 131 may be adopted to control to display only an original image outputted from either the composition/masking processing circuit 108H or the composition/masking processing circuit 108S which matches setting (the SDTV or the HDTV) performed in the item ‘Decode Type’ in the column ‘Decode’ on the setting screen shown in FIG. 9, so that a predetermined image such as a black screen, a blue screen and an error display instead of the other multi image outputted from the circuit which does not match the setting without displaying the other multi image is displayed.

According to the processing at step CFLW13 shown in FIG. 13, the original image is outputted as shown in FIG. 14C. The CPU 131 may be adopted to report that the image recorded in the peripheral device or the like (and not the image being observed) is displayed by lighting a predetermined LED provided on the operating device or by displaying a message directing that the original image is displayed while the original image is displayed. That enables the user to easily recognize that an image recorded in the peripheral device or the like is displayed (on the display unit such as a monitor).

As shown in FIG. 14C, the original images can be switched for each page (a screen of the original image) in response to pressing of the switch-to-next-page key (e.g., PageUp key on the keyboard 5 or the like) or switch-to-previous-page key (e.g., PageDown key on the keyboard 5 or the like) of the operating device, for example.

When the CPU 131 detects that either the switch-to-next-page key or the switch-to-previous-page key is pressed and a page switching direction is issued for the original images (step CFLW14 shown in FIG. 13), the processing to display the Wait Screen (step CFLW15 shown in FIG. 13) is preformed and also the original image on the specified page is generated and outputted while the Wait Screen is being displayed (step CFLW16 shown in FIG. 13). The CPU 131 is not limited to generate the original images on the specified pages one by one as in the processing at step CFLW9 shown in FIG. 13 and may output the original image as it is in response to the specification of the generated original image. If the CPU 131 detects any of the cases where a direction to change the page is issued when there is only one page, where a switch-to-previous-page direction is issued when there is no previous page, or where a switch-to-next-page direction is issued when there is no page next, pressing of the key on the keyboard 5 or the like may be described and an alert with a beep or an error display may be performed. The CPU 131 may causes to display the page number (for each of the multi images) on the upper right corner or the like on the original images.

When the CPU 131 detects that a predetermined key (e.g., Backspace key or ESC key on the keyboard 5 or the like) of the operating device is pressed and a direction to return to the previous screen is issued (step CFLW17 shown in FIG. 13), it controls again to display the Wait Screen according to the processing at step CFLW5 shown in FIG. 13 and then output the multi image according to the processing at step CFLW6 shown in FIG. 13.

When the CPU 131 detects that a predetermined key (e.g., an arrow key or the like on the keyboard 5) and a confirmation key (e.g., ENTER key of the keyboard 5 or the like) of the operating device are pressed and an image file is directly selected and confirmed (step CFLW18 shown in FIG. 13), it performs the processing to display the Wait Screen according to the processing at step CFLW12 shown in FIG. 13 and also output an original image of the image file according to the processing at step CFLW13 shown in FIG. 13.

When the CPU 131 detects that the directory name and the fine name are displayed without being selected and confirmed and that a predetermined key of the operating device (e.g., Backspace key or ESC key of the keyboard 5 or the like) is pressed and a direction to return to the previous screen is issued (step CFLW20 shown in FIG. 13), it finishes the procedure for displaying a still image recorded in the peripheral device or the like.

Now, the processing performed when a key or a switch with either a release function or a capture function added (hereinafter they will be collectively referred to as a record direction key) among keys and switches of the operating devices is pressed will be described. It is assumed that an endoscope composite image (e.g., such an image as shown in FIG. 7) whose display size (‘Mon size’ in the setting screen shown in FIG. 8) is set to 16:9 is recorded in the description below. The description for FIG. 15A to FIG. 15E shown below mainly describes the processing and the operation performed when a key or a switch to which any of the abovementioned ‘Release 1’ to ‘Release4’ is allocated as the record direction key.

First, the CPU 131 of the main controlling unit 75 detects whether the record direction key of the operating device is pressed or not. When the CPU 131 detects that the record direction key of the operating device is pressed (step BBFLW1 shown in FIG. 15A), it performs the processing to stop the image and the still image processing that is further performed on the image stopped by the first processing (step BBFLW2 shown in FIG. 15A).

Specifically, as the still image processing at step BBFLW2 shown in FIG. 15A, the CPU 131 performs the processing to cause the freeze circuit 96 to generate a freeze image and perform pre-freeze processing, and then control the post-stage image processing circuit 98 to calculate an average of IHb in the still image, control the graphic circuit 106H to temporarily change the hemoglobin index 322A according to the calculated result, control the graphic circuit 106H to temporarily fix (freeze) the display of the time information 308, control the graphic circuit 106H to temporarily delete the cursor 319, controls the graphic circuit 169 of the expansion controlling units 77A and 77B to temporarily fix (freeze) or delete an image or the like, and control the composition/masking processing circuits 108H and 108S to temporarily delete the group of thumbnail images 326A. As a result of the control and processing, both of the endoscope composite image in the SDTV that is outputted from the composition/masking processing circuit 108S and the endoscope composite image in the HDTV that is outputted from the composition/masking processing circuit 108H are in a still state. When a freeze image (or an S freeze image) has been displayed as the endoscope image 301 (or the endoscope image 302) by a switch with a freeze function (or an S freeze function) allocated to the operating device, all the processing except for the processing related to the time information 308, the processing related to the cursor 319, the control on the graphic circuit 169 and the processing related to the group of thumbnail images 326A from the processing performed at step BBFLW2 shown in FIG. 15A. The processing performed at step BBFLW2 shown in FIG. 15A will be referred to as a still image processing below and the drawings.

Then, the CPU 131 controls the selector 125D via the memory controlling circuit 125A of the controller/selector 125, so that the endoscope composite images are outputted. The images are outputted from the composition/masking processing circuit 108S and then stored in the memory 125C, to the memory 126 frame by frame (or line by line) (step BBFLW3 shown in FIG. 15A). Specifically, the memory controlling circuit 125A controls the composition/masking processing circuit 108S and the memory 125C under control of the CPU 131, and causes the memory 125C to store a frame (or a line) of the endoscope composite image in the SDTV that is outputted from the composition/masking processing circuit 108S and also causes the memory 125C to perform frequency conversion on the endoscope composite image from 13.5 MHz to 100 MHz, and then output the endoscope composite image to the memory 126 frame by frame (or line by line).

Further, the CPU 131 outputs the record direction signal or the record direction command to the peripheral device that is selected and set in the sub-item ‘Peripheral Device’ of the items ‘Release1’, ‘Release2’, ‘Release3’ and ‘Release4’ in the column ‘SDTV’ in the setting screen shown in FIG. 8. Specifically, when any of the peripheral devices shown in FIG. 3A is selected in the item ‘Peripheral Device’, the CPU 131 outputs the record direction signal or the record direction command to the selected peripheral device via either the signal line 142a or the signal line 143a.

After performing the processing at step BBFLW3 shown in FIG. 15A, the CPU 131 causes the thumbnail image generating section 105S to generate a thumbnail image and also set the display position for the thumbnail image (step BBFLW4 shown in FIG. 15A). It is assumed that the thumbnail image has not been displayed in the screen when the processing at step BBFLW4 shown in FIG. 15A is performed.

When a peripheral device that can support images of both display sizes of 4:3 and 16:9 is set in the item ‘Peripheral Device’ (step BBFLW5 shown in FIG. 15A), the CPU 131 detects whether the peripheral device supports the recorded image display mode, which is a mode for recording an image that almost matches the still image displayed on a monitor when it is directed to record, or not. When the peripheral device that can support images of both display sizes of 4:3 and 16:9 and also support the recorded image display mode is set in the item ‘Peripheral Device’ (step BBFLW7 shown in FIG. 15A), the CPU 131 performs the control and the processing shown in FIG. 15D. When the peripheral device that can support images of both display sizes of 4:3 and 16:9 but cannot support the recorded image display mode is set (step BBFLW7 shown in FIG. 15A), the CPU 131 performs the control and the processing shown in FIG. 15E to be described later.

When the peripheral device that can only support the display size of 4:3 is set in the item ‘Peripheral Device’ (step BBFLW5 shown in FIG. 15A), the CPU 131 further detects whether the peripheral device supports the recorded image display mode or not. When the peripheral device that can support only the image of the display size 4:3 and also support the recorded image display mode is set in the item ‘Peripheral Device’ (step BBFLW6 shown in FIG. 15A), the CPU 131 performs the control and the processing shown in FIG. 15B to be described later. When the peripheral device that can support only the image of the display size 4:3 but cannot support the recorded image display mode is set in the item ‘Peripheral Device’ (step BBFLW6 shown in FIG. 15A), the CPU 131 performs the control and the processing shown in FIG. 15C to be described later.

The printer 202B1, the VTR 203B1, the filing device 204B1 and the photographing device 205B1 among the peripheral devices shown from FIG. 3A to FIG. 3E can support only the image of the display size of 4:3 and also support the recorded image display mode (they are appliance that can record an image which almost matches a still image displayed on the monitor 201B1 or the monitor 201C1). Thus, when any of the printer 202B1, the VTR 203B1, the filing device 204B1 and the photographing device 205B1 shown in FIG. 3B is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15B to be described later.

The printer 202B2, the VTR 203B2, the filing device 204B2 and the photographing device 205B2 among the peripheral devices shown from FIG. 3A to FIG. 3E can support both of the images of the display sizes of 4:3 and 16:9 and also support the recorded image display mode (they are appliance that can record an image which almost matches a still image displayed on the monitor 201B2 or the monitor 201C2). Thus, when any of the printer 202B2, the VTR 203B2, the filing device 204B2 and the photographing device 205B2 shown in FIG. 3B is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15D to be described later.

The printer 202C1, the VTR 203C1, the filing device 204C1, the photographing device 205C1, the endoscope form detecting device 206C1 and the ultrasonic device 207C1 among the peripheral devices shown from FIG. 3A to FIG. 3E can support only the image of the display size of 4:3 and also support the recorded image display mode (they are appliance that can record an image which almost matches a still image displayed on the monitor 201C1 or the monitor 201B1). Thus, when any of the printer 202C1, the VTR 203C1, the filing device 204C1 and the photographing device 205C1, the endoscope form detecting device 206C1 and the ultrasonic device 207C1 shown in FIG. 3C is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15B to be described later.

The printer 202C2, the VTR 203C2, the filing device 204C2, the photographing device 205C2, the endoscope form detecting device 206C2 and the ultrasonic device 207C2 among the peripheral devices shown from FIG. 3A to FIG. 3E can support both of the images of the display sizes of 4:3 and 16:9 and also support the recorded image display mode (they are appliance that can record an image which almost matches a still image displayed on the monitor 201C2 or the monitor 201B2). Thus, when any of the printer 202C2, the VTR 203C2, the filing device 204C2 and the photographing device 205C2, the endoscope form detecting device 206C2 and the ultrasonic device 207C2 shown in FIG. 3C is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15D to be described later.

The printer 202D1, the filing device 204D1, the photographing device 205D1, the optical recording device 208D1 and the HID 209D1 among the peripheral devices shown from FIG. 3A to FIG. 3E can support only the image of the display size of 4:3 but cannot support the recorded image display mode. Thus, when any of the printer 202D1, the filing device 204D1, the photographing device 205D1, the optical recording device 208D1 and the HID 209D1 shown in FIG. 3D is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15C to be described later.

The printer 202D2, the filing device 204D2, the photographing device 205D2, the optical recording device 208D2 and the HID 209D2 among the peripheral devices shown from FIG. 3A to FIG. 3E can support both of the images of the display sizes of 4:3 and 16:9 but cannot support the recorded image display mode. Thus, when any of the printer 202D2, the filing device 204D2, the photographing device 205D2, the optical recording device 208D2 and the HID 209D2 shown in FIG. 3D is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15E to be described later. The PC card 167 and the memory card 168 shown in FIG. 2D can also support both of the images of the display sizes of 4:3 and 16:9 but cannot support the recorded image display mode. Thus, when either the PC card 167 or the memory card 168 is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15E to be described later.

The printer 202E1, the filing device 204E1, the photographing device 205E1, and the optical recording device 208E1 among the peripheral devices shown from FIG. 3A to FIG. 3E can support only the image of the display size of 4:3 but cannot support the recorded image display mode. Thus, when any of printer 202E1, the filing device 204E1, the photographing device 205E1, and the optical recording device 208E1 shown in FIG. 3E is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15C to be described later.

The printer 202E2, the filing device 204E2, the photographing device 205E2, and the optical recording device 208E2 among the peripheral devices shown from FIG. 3A to FIG. 3E can support both of the images of the display sizes of 4:3 and 16:9 but cannot support the recorded image display mode. Thus, when any of the printer 202E2, the filing device 204E2, the photographing device 205E2, and the optical recording device 208E2 shown in FIG. 3E is selected and set in a sub-item ‘Peripheral Device’ of the items ‘Release1’, ‘Release2’, ‘Release3’, and ‘Release4’ in the column ‘HDTV’ on the setting screen shown in FIG. 8, the CPU 131 performs the control and the processing shown in FIG. 15E to be described later.

Now, the processing shown in FIG. 15B, which is the processing performed following to the procedure shown in FIG. 15A (and the processing associated with the procedure shown in FIG. 15A) will be described below.

The CPU 131 detects whether an S freeze image is outputted as the endoscope image 302 or not (step BBFLW11 shown in FIG. 15B). When the CPU 131 detects that an S freeze image is outputted as the endoscope image 302, it controls the composition/masking processing circuit 108H and the image I/O processing section 121, and generates the S freeze image to be recorded (referred to as an S freeze image to be recorded) in the display size of 4:3 as shown in the screen 1098 shown in FIG. 19B and outputs the S freeze image to the D/A 110H or the image I/O processing section 121 (step BBFLW12 shown in FIG. 15B), and also causes the memory 126 to store the S freeze image (to be recorded) (step BBFLW13 shown in FIG. 15B). When the CPU 131 detects that an S freeze image is not outputted as the endoscope image 302 (step BBFLW11 shown in FIG. 15B), the CPU 131 keeps on performing the processing at step BBFLW19 shown in FIG. 15B to be described later.

Specifically, the memory controlling circuit 125A controls the composition/masking processing circuit 108H, the memory 112H and the memory 125B under control of the CPU 131 and causes the memory 125B to store a frame (a line) of the S freeze image to be recorded that is outputted from the composition/masking processing circuit 108 and also causes the memory 125B to perform frequency conversion on the S freeze image to be recorded from 74 MHz to 100 MHz, and then output the S freeze image to be recorded to the memory 126 frame by frame (or line by line). The CPU 131 displays the S freeze image to be recorded on the display unit of a monitor or the like for a time period according to the time set in the item ‘HDTV’ in the column ‘Release Time’ by causing the S freeze image that is outputted to the memory 126 to be also outputted to a monitor.

FIG. 16 shows an example of an image region stored in the memory 126. The X coordinate in the image region is a count in a horizontal direction that is generated based on a horizontal synchronizing signal and a clock for image processing with the leftmost value of zero incremented toward right. The Y coordinate in the image region is a count in a vertical direction that is generated based on a horizontal synchronizing signal and a vertical synchronizing signal with the top value of zero incremented toward bottom.

The processing performed by the CPU 131 or the like before and after the image in the image region shown in FIG. 16 is stored in the memory 126 will be shown below.

The CPU 131 sets a rectangular area surrounded by four coordinates of (wstarth, wstartv), (wendh, wstartv), (wstarth, wendv) and (wendh, wendv) which are stored in at least one of the ROM 135 and the backup RAM 137 of the main controlling unit 75, the ROM 154 and the backup RAM 155 of the expansion controlling unit 77A, or the ROM 154 of the expansion controlling unit 77B, for example, as table data as an image region. The four coordinates of (wstarth, wstartv), (wendh, wstartv), (wstarth, wendv) and (wendh, wendv) which are stored as table data differ according to the conditions such that an image in the SDTV to be inputted is outputted, that the image in the HDTV to be inputted is outputted as an image of the display size of 4:3, and that the image in the HDTV to be inputted is outputted as an image of the display size of 16:9. FIG. 4 shows an example of each display size.

The CPU 131 performs the processing at step BBFLW13 shown in FIG. 15B, and then causes the thumbnail image generating section 105H to generate the thumbnail image, and also sets the display position of the thumbnail image 326 in the group of the thumbnail image 326A (step BBFLW14 shown in FIG. 15B). It is assumed that the thumbnail image is not displayed in the screen when the processing at step BBFLW14 shown in FIG. 15B is performed.

FIG. 17 shows an example of the thumbnail image generated by the thumbnail generating sections 105S and 105H and the image region that provides a standard when the thumbnail image is generated.

The X coordinate in the image region is a count in a horizontal direction that is generated based on a horizontal synchronizing signal and a clock for image processing with the leftmost value of zero incremented toward right. The Y coordinate in the image region is a count in a vertical direction that is generated based on a horizontal synchronizing signal and a vertical synchronizing signal with the top value of zero incremented toward bottom.

The processing performed when a thumbnail image is generated by the thumbnail image generating sections 105S and 105H will be shown below.

The CPU 131 sets a rectangular area surrounded by four coordinates of (mrstarth, mrstartv), (mrendh, mrstartv), (mrstarth, mrendv) and (mrendh, mrendv) which are stored in at least one of the ROM 135 and the backup RAM 137 of the main controlling unit 75, the ROM 154 and the backup RAM 155 of the expansion controlling unit 77A, or the ROM 154 of the expansion controlling unit 77B, for example, as table data as an image region in the thumbnail image. Then, the CPU 131 changes the rectangular area according to the image size (Medium, Simi-Full, or Full) and the type of the endoscope 2A (or 2B), and also generates a thumbnail image from an image included in the rectangular area. The reduction rate applied to generate the thumbnail image depends on the image size or the type of the endoscope 2a and 2B. The reduction rate may be any of those stored as ‘table data’ together with the four coordinates of (mrstarth, mrstartv), (mrendh, mrstartv), (mrstarth, mrendv) and (mrendh, mrendv). For the four coordinates of (mrstarth, mrstartv), (mrendh, mrstartv), (mrstarth, mrendv) and (mrendh, mrendv) which are stored as the table data, different values may be set according to the combination of the image size to be inputted, the type of the endoscope (or CCD) and the display size of the image to be outputted. A user may change the reduction rate in generating the thumbnail image to a desired value on the setting screen or the like (not shown).

Then, the CPU 131 outputs a record directing signal or a record directing command to a peripheral device set in ‘Peripheral device’ that is one of the subitems included in the items ‘Release1’, ‘Release2’, ‘Release3’ and ‘Release4’ in the ‘HDTV’ column of the setting screen shown in FIG. 8 via the signal line 142a or the signal line 143a and records an S freeze image to be recorded (step BBFLW15 shown in FIG. 15B).

The CPU 131 detects whether a period set in the ‘HDTV’ in the ‘Release Time’ column of the setting screen shown in FIG. 8 has elapsed or not.

When the CPU 131 detects that the period set in the ‘HDTV’ item in the ‘Release Time’ column of the setting screen shown in FIG. 8 has elapsed (step BBFLW16 shown in FIG. 15B), the CPU 131 successively performs processing directed in step BBFLW19 shown in FIG. 15B to be described later. When the CPU 131 detects that the period set in the ‘HDTV’ item in the ‘Release Time’ column of the setting screen shown in FIG. 8 has not elapsed (step BBFLW16 shown in FIG. 15B), the CPU 131 repeatedly performs detection of whether the period (HDTV release period) set in the ‘HDTV’ item in the ‘Release Time’ column has elapsed or not (step BBFLW16 shown in FIG. 15B).

The CPU 131 causes to display the S freeze image to be recorded on the monitor for only the period set in the ‘HDTV’ item in the ‘Release Time’ column of the setting screen shown in FIG. 8. Subsequently, once the period has elapsed, the CPU 131 controls the composition/masking processing circuit 108H so that, for example, a freeze image to be recorded whose display size is 4:3 (hereinafter referred to as freeze image to be recorded) such as that depicted on a screen 1903 shown in FIG. 19A or a screen 1909 shown in FIG. 19B is generated and the same to the D/A 110H or the image I/O processing circuit 121 (step BBFLW17 shown in FIG. 15B) is outputted, and the freeze image (to be recorded) in the memory 126 (step BBFLW18 shown in FIG. 15B) is stored.

More specifically, based on the control by the CPU 131, the memory controller 125A controls the composition/masking processing circuit 108H and the memory 125B in order to store only one frame's (or one line's) worth of the freeze image to be recorded outputted from the composition/masking processing circuit 108H in the memory 125B, subjects the freeze image to be recorded to frequency conversion at the memory 125B from 74 MHz to 100 MHz frequency conversion, and sequentially outputs the freeze image to be recorded to the memory 126 one frame (or one line) at a time. Furthermore, by also outputting the freeze image to be recorded to the monitor, the CPU 131 displays the freeze image to be recorded for exactly the period set in the ‘HDTV’ item in the ‘Release Time’ column.

It is assumed that the processing for generating an image with a 4:3 display size (freeze image to be recorded) from an image with a 16:9 display size (HDTV endoscope composite image), which is performed as the processing of step BBFLW17 shown in FIG. 15B, is the same as the abovementioned processing of step BBFLW12 shown in FIG. 15B.

After performing the processing of BBFLW18 shown in FIG. 15B, the CPU 131 generates a thumbnail image at the thumbnail image generating section 105H, and sets a display position of the thumbnail image as a thumbnail image 326 among the group of thumbnail images 326A for the thumbnail image (step BBFLW19 shown in FIG. 15B). However, it is assumed that the thumbnail is not yet displayed within the screen when the processing of step BBFLW19 shown in FIG. 15B is performed.

It is assumed that the processing related to thumbnail image generation performed as the processing of step BBFLW18 shown in FIG. 15B is the same as the abovementioned processing of step BBFLW14 shown in FIG. 15B.

Then, the CPU 131 outputs a record directing signal or a record directing command to the peripheral device set in ‘Peripheral device’ that is one of the subitems included in items ‘Release1’, ‘Release2’, ‘Release3’ and ‘Release4’ in the ‘HDTV’ column of the setting screen shown in FIG. 8 via the signal line 142a or the signal line 143a so that a freeze image (step BBFLW20 shown in FIG. 15B) is stored.

The CPU 131 further detects whether the period set in the ‘HDTV’ item in the ‘Release Time’ field of the setting screen shown in FIG. 8 has elapsed or not.

Then, when the CPU 131 detects that the period set in the ‘HDTV’ item in the ‘Release Time’ column of the setting screen shown in FIG. 8 has elapsed (step BBFLW21 shown in FIG. 15B), the CPU 131 successively performs processing directed in step BBFLW22 shown in FIG. 15B to be described later. When the CPU 131 detects that the period set in the ‘HDTV’ item in the ‘Release Time’ column of the setting screen shown in FIG. 8 has not elapsed (step BBFLW21 shown in FIG. 15B), the CPU 131 repeatedly performs detection of whether the period (HDTV release period) set in the ‘HDTV’ item in the ‘Release Time’ column of the setting screen shown in FIG. 8 has elapsed or not (step BBFLW21 shown in FIG. 15B).

Subsequently, the CPU 131 releases still image processing by the processing described below, and controls the composition/masking processing circuit 108H, so that an HDTV endoscope composite image such as that depicted on a screen 1910 shown in FIG. 19B (step BBFLW22 shown in FIG. 15B) is generated and outputted.

More specifically, the CPU 131 outputs a moving image as the endoscope image 301, and performs processing that newly outputs, for example, among the thumbnail images, a thumbnail image generated in step BBFLW4 shown in FIG. 15A, step BBFLW14 shown in FIG. 15B and step BBFLW19 shown in FIG. 15B as the thumbnail images 326. (As a result of the abovementioned processing, for example, the number of thumbnail images 326 in the group of thumbnail images 326A depicted on the screen 1910 shown in FIG. 19B is increased to 3).

The CPU 131 performs processing for clearing the S freeze image displayed as the endoscope image 302 upon the input of the record directing key and the group of image related information 302A in conjunction with the abovementioned processing of step BBFLW22 shown in FIG. 15B.

When the CPU 131 detects upon the input of the record directing key that an image or the like had been outputted from the graphic circuit 169 of the expansion controlling unit 77A and (or) 77B, the CPU 131 controls the graphic circuit 169 of the expansion controlling unit 77A and (or) 77B to perform processing for resuming output of a portion of or an entirely of the image or the like. Furthermore, the CPU 131 controls the graphic circuit 106H so that the processing for: adding 1 to the value of D.F311 (or SCV309 or CVP310) of the group of observe information 300 is performed and the same; changing the display content of the hemoglobin index 322A (e.g., to ‘IHb=---’); releasing fixation of the display of time information 308; is released and the cursor 319, in conjunction with the abovementioned processing is redisplayed. The CPU 131 also causes to interrupt generation of the freeze image at the freeze circuit 96, and performs processing for outputting a moving image at the composition/masking processing circuit 108H in conjunction with the abovementioned processing. The CPU 131 further controls the synchronization circuit 101S and the memory 104S so that a freeze image is generated, and performs processing for outputting the freeze image to the composition/masking processing circuit 108S in conjunction with the abovementioned processing. Consequently, the CPU 131 continuously causes to output SDTV still images.

Then, when the CPU 131 detects that the period set in the ‘SDTV’ item in the ‘Release Time’ column has elapsed (step BBFLW23 shown in FIG. 15B), the CPU 131 releases still image processing by the same processing as in step BBFLW22 shown in FIG. 15B (step BBFLW24 shown in FIG. 15B), and performs processing for interrupting the generation of the freeze image by controlling the synchronization circuit 101S and the memory 104S.

According to the series of processing shown in (FIG. 15A and) FIG. 15B described above, a transition of screens displayed on a monitor or the like takes place as shown in, for example, FIG. 19A or FIG. 19B.

First, the screen transition shown in FIG. 19A will be described.

When a user is in the middle of an observation or when the user releases a freeze direction, a moving image of a subject currently under observation is displayed as the endoscope image 301 (screen 1901 shown in FIG. 19A). When the user operates a key, a switch or the like having a freeze function and a freeze direction is issued, a freeze image at the timing of the direction is displayed as the endoscope image 301 (screen 1902 shown in FIG. 19A).

Then, when the record directing key is operated by the user and a release direction or a capture direction is issued from either of the states of the screen 1901 or the screen 1902 shown in FIG. 19A, a freeze image to be recorded 401 including the endoscope image 301 and having a 4:3 display size is displayed as an image currently being recorded in place of an image with a 16:9 display size (screen 1903 shown in FIG. 19A). At this point, the group of observe information 300 and the group of image related information 301A are collectively displayed on the left hand side of the freeze image to be recorded 401. In addition, (due to, for example, processing performed by the graphic circuit 106H), information corresponding to the light equipment type 325A (for example, ‘Nr’ in the case where the light equipment type 325A is ‘Normal’) in the group of image related information 301A is displayed as light source information 401a at a corner of the freeze image to be recorded 401 (screen 1903 shown in FIG. 19A). In other words, among the screen 1903 displayed on a monitor or the like, an image having a 4:3 display size such as that shown as the freeze image to be recorded 401 is recorded onto the peripheral device.

Once recording of the freeze image to be recorded 401 to the peripheral device is concluded, a moving image of the subject currently under observation is once again displayed as the endoscope image 301, accompanied by a display of the thumbnail image 326 of the endoscope image 301 in the freeze image to be recorded 401 (screen 1904 shown in FIG. 19A).

Next, the screen transition shown in FIG. 19B will be described.

When a user is in the middle of an observation or when the user releases an S freeze direction, a moving image of a subject currently under observation is displayed as the endoscope image 301 (screen 1905 shown in FIG. 19B). At the screen 1905 shown in FIG. 19B, when a key, a switch or the like having an S freeze function is operated by the user and an S freeze direction is issued, an S freeze image at the timing of the direction is displayed as the endoscope image 302, and at the same time, the moving image of the subject currently under observation is continually displayed as the endoscope image 301 (screen 1906 shown in FIG. 19B). Further, at the screen 1906 shown in FIG. 19B, when the user operates a key, a switch or the like having a freeze function and a freeze direction is issued, together with the S freeze image, a freeze image at the timing of the direction is displayed as the endoscope image 301 (screen 1907 shown in FIG. 19B).

Then, when the record directing key is operated by the user and a release direction or a capture direction is issued from either of the states of the screen 1906 or the screen 1907 shown in FIG. 19B, an S freeze image to be recorded 402 including the endoscope image 302 and having a 4:3 display size is displayed as an image currently being recorded in place of an image with a 16:9 display size (screen 1908 shown in FIG. 19B). At this point, the group of observe information 300 and the group of image related information 302A are collectively displayed on the left hand side of the freeze image to be recorded 402. In addition, (due to, for example, processing performed by the graphic circuit 106H), information corresponding to the light equipment type 325B (for example, ‘Nr’ in the case where the light equipment type 325B is ‘Normal’) in the group of image related information 302A is displayed as light source information 402a at a corner of the freeze image to be recorded 402 (screen 1908 shown in FIG. 19B). In other words, among the screen 1908 displayed on a monitor or the like, an image having a 4:3 display size such as that shown as the freeze image to be recorded 402 is recorded onto the peripheral device. Once recording of the S freeze image to be recorded 402 to the peripheral device is concluded, a freeze image to be recorded 401 including the endoscope image 301 and having a 4:3 display size is displayed as an image currently being recorded in place of the S freeze image to be recorded (screen 1909 shown in FIG. 19B). At this point, the group of observe information 300 and the group of image related information 301A are collectively displayed on the left hand side of the freeze image to be recorded 401. In other words, among the screen 1909 displayed on a monitor or the like, an image having a 4:3 display size such as that shown as the freeze image to be recorded 401 is recorded onto the peripheral device together with the abovementioned screen 1908.

Once recording of the freeze image to be recorded 401 and the S freeze image to be recorded 402 to the peripheral device is concluded, the moving image of the subject currently under observation is once again displayed as the endoscope image 301, and as the plurality of thumbnail images 326, a thumbnail image of the endoscope image 301, a thumbnail image of the endoscope image 302, and thumbnail images originally existing in the group of thumbnail images 326A are displayed together therewith (screen 1910 shown in FIG. 19B). More specifically, for example, in the case of the screen 1910 shown in FIG. 19B, thumbnail images originally existing in the group of thumbnail images 326A at the time of the screen 1905 shown in FIG. 19B are displayed at the very top, the thumbnail image of the endoscope image 302 is displayed thereunder, and the thumbnail image of the endoscope image 301 is displayed further thereunder.

In the present embodiment, as the group of thumbnail images 326A, it is assumed that a maximum of four images are displayed (in the order in which record directions were issued) as the thumbnail images 326.

Now, each processing (as well as processing related to each processing) shown in FIG. 15C that is processing performed following each processing shown in FIG. 15A will be described.

After storing the endoscope composite image with a 16:9 display size in the memory 109H, the CPU 131 reads the endoscope composite image from the memory 109H and outputs the same to the monitor via the signal line 111Ha or the signal line 121a (step BBFLW41 shown in FIG. 15C). As a result of the CPU 131 performing the processing of step BBFLW41 shown in FIG. 15C, neither of an S freeze image to be recorded that is generated in the processing of step BBFLW42 shown in FIG. 15C nor a freeze image to be recorded that is generated in the processing of step BBFLW45 shown in FIG. 15C are displayed on the monitor. It is assumed that the abovementioned endoscope composite image includes both a freeze image as the endoscope image 301 and an S freeze image as the endoscope image 302.

Then, the CPU 131 controls the composition/masking processing circuit 108H so that an S freeze image to be recorded by the same processing as that of abovementioned step BBFLW12 shown in FIG. 15B (step BBFLW42 shown in FIG. 15C) is generated, and the S freeze image to be recorded in the memory 126 (step BBFLW43 shown in FIG. 15C) is stored.

After performing the processing of step BBFLW43 shown in FIG. 15C, the CPU 131 generates a thumbnail image and sets a display position thereof by the same processing as that of abovementioned step BBFLW14 shown in FIG. 15B (step BBFLW44 shown in FIG. 15C).

The CPU 131 controls the composition/masking processing circuit 108H and generates a freeze image to be recorded by the same processing as that of abovementioned step BBFLW17 shown in FIG. 15B (step BBFLW45 shown in FIG. 15C), and stores the freeze image to be recorded in the memory 126 (step BBFLW46 shown in FIG. 15C).

After performing the processing of step BBFLW46 shown in FIG. 15C, the CPU 131 generates a thumbnail image and sets a display position thereof by the same processing as that of abovementioned step BBFLW19 shown in FIG. 15B (step BBFLW47 shown in FIG. 15C).

Then, the CPU 131 releases still image processing by the same processing as step BBFLW22 and step BBFLW24 shown in FIG. 15B (step BBFLW48 shown in FIG. 15C), and also releases reading from the memory 109H. Consequently, the CPU 131 causes to output a moving image as the endoscope composite image 301.

Subsequently, the CPU 131 (and the CPU 151) performs processing for compressing and recording the freeze image to be recorded, the S freeze image to be recorded and the thumbnail images stored in the memory 126 (step BBFLW49 shown in FIG. 15C). Details of the processing of step BBFLW49 shown in FIG. 15C will be described later as a description regarding processing of step BBFLW84 shown in FIG. 15E.

According to the series of processing shown in (FIG. 15A and) FIG. 15C described above, a transition of screens displayed on a monitor or the like takes place as shown in, for example, FIG. 19C or FIG. 19D.

First, the screen transition shown in FIG. 19C will be described.

When the record directing key is operated by the user and a release direction or a capture direction is issued from the state of a screen 1911 which is approximately the same display state as the screen 1907 shown in FIG. 19B, an S freeze image to be recorded 402 including the endoscope image 302 is recorded onto the peripheral device (screen 1912 shown in FIG. 19C) and, consecutively, a freeze image to be recorded 401 including the endoscope image 301 is recorded onto the peripheral device (screen 1913 shown in FIG. 19C). Prior to the recording of the images on the screen 1912 and the screen 1913 shown in FIG. 19C, processing of abovementioned step BBFLW49 shown in FIG. 15C is performed to release freezing of an image being outputted to the monitor. Consequently, a moving image of the subject currently under observation is displayed as the endoscope image 301, and as the plurality of thumbnail images 326, a thumbnail image of the endoscope image 301, a thumbnail image of the endoscope image 302, and thumbnail images originally existing in the group of thumbnail images 326A are displayed together therewith (screen 1914 shown in FIG. 19C). In other words, images on the screen 1912 and the screen 1913 which differ from any of the images on the screens 1911 and 1914 displayed on the monitor or the like are recorded together onto the peripheral device.

Once recording of the freeze image to be recorded 401 and the S freeze image to be recorded 402 to the peripheral device is concluded, in addition to the display content of the screen 1914, a record finish notification message 501 for notifying that recording of each image has been concluded (or an error occurred during recording) is displayed (screen 1915 shown in FIG. 19C).

Next, the screen transition shown in FIG. 19D will be described.

In addition to the screen 1907 shown in FIG. 19B, from a state of a screen 1916 that is a screen in which the endoscope form image 502 outputted from the expansion controlling unit 77B, the zoom control information 503, and the PinP image 504 outputted from the expansion controlling unit 77A are displayed together, when the record directing key is operated by the user and a release direction or a capture direction is issued, an S freeze image to be recorded 402 including the endoscope image 302 is recorded onto the peripheral device (screen 1917 shown in FIG. 19D), and consecutively, a freeze image to be recorded 401 including the endoscope image 301 is recorded onto the peripheral device (screen 1918 shown in FIG. 19D). In other words, images on the screen 1916 and the screen 1919 which differ from any of the images on the screen 1917 and the screen 1918 displayed on the monitor or the like are recorded together onto the peripheral device.

In the present embodiment, it is assumed that the endoscope form image 502 and the zoom control information 503 displayed as a state of the screen 1916 are recorded together with the freeze image to be recorded 401. In the present embodiment, display positions of the endoscope form image 502 and the zoom control information 503 may be changed based on the control of the CPU 131 by, for example, the graphic circuit 106H or by the graphic circuit 169 of the expansion controlling unit 77B. Furthermore, although the present embodiment is arranged so that the PinP image 504 displayed as a state of the screen 1916 is not considered an object to be recorded, the present embodiment is not limited to this arrangement. More specifically, for example, whether or not the endoscope form image 502, the zoom control information 503 and the PinP image 504 are to be recorded can be arranged so as to be individually changeable on a setting screen, not shown.

Prior to recording of the screen 1917 and the screen 1918 shown in FIG. 19D, the freezing of the image outputted to the monitor is released and a moving image of the subject currently under observation is displayed as the endoscope image 301, and the endoscope form image 502, the zoom control information 503 and the PinP image 504 are displayed in a state in which, for example, positions thereof are moved so as to avoid overlapping the endoscope image 301 (screen 1919 shown in FIG. 19D). Display positions of the endoscope form image 502 and the zoom control information 503 may be changed based on the control of the CPU 131 by, for example, the graphic circuit 106H or by the graphic circuit 169 of the expansion controlling unit 77B. On the screen 1919 shown in FIG. 19D, as the plurality of thumbnail images 326, a thumbnail image of the endoscope image 301, a thumbnail image of the endoscope image 302, and thumbnail images originally existing in the group of thumbnail images 326A are displayed together with the respective abovementioned images.

Once recording of the freeze image to be recorded 401 and the S freeze image to be recorded 402 to the peripheral device is concluded, in addition to the display content of the screen 1919, a record finish notification message 501 for notifying that recording of each image has been concluded (or an error occurred during recording) is displayed (screen 1920 shown in FIG. 19D).

Priorities for the endoscope form image 502, the zoom control information 503 and the PinP image 504 such as which image will be displayed foremost (or backmost) when displayed with the image superimposed upon each other can be arranged to be set on a setting screen or the like, not shown. A portion or an entirely of the endoscope form image 502, the zoom control information 503 and the PinP image 504 may be arranged to be erased at, for example, a screen during finishing of recording or after finish of recording (the screen 1919 or the screen 1920).

Now, each processing (as well as processing related to each processing) shown in FIG. 15D that is processing performed following each processing shown in FIG. 15A will be described.

The CPU 131 causes to store an endoscope composite image with a 16:9 display size in the memory 126 (step BBFLW61 shown in FIG. 15D).

Next, by the same processing as that of abovementioned step BBFLW14 shown in FIG. 15B, the CPU 131 generates a thumbnail image and sets a display position thereof (step BBFLW62 shown in FIG. 15D).

Then, the CPU 131 outputs a record directing signal or a record directing command to the peripheral device set in ‘Peripheral device’ that is one of the subitems included in items ‘Release1’, ‘Release2’, ‘Release3’ and ‘Release4’ in the ‘HDTV’ column of the setting screen shown in FIG. 8 via the signal line 142a or the signal line 143a and records the endoscope composite image with a 16:9 display size (step BBFLW63 shown in FIG. 15D).

When the CPU 131 detects that the period set in the ‘HDTV’ item in the ‘Release Time’ column of the setting screen shown in FIG. 8 has elapsed (step BBFLW64 shown in FIG. 15D), the CPU 131 successively performs processing directed in step BBFLW65 shown in FIG. 15D to be described later. When the CPU 131 detects that the period set in the ‘HDTV’ item in the ‘Release Time’ column of the setting screen shown in FIG. 8 has not elapsed (step BBFLW64 shown in FIG. 15D), the CPU 131 repeatedly performs detection of whether the period (HDTV release period) set in the ‘HDTV’ item in the ‘Release Time’ column of the setting screen shown in FIG. 8 has elapsed or not (step BBFLW64 shown in FIG. 15D).

Subsequently, the CPU 131 releases still image processing by performing the same processing as the abovementioned step BBFLW22, step BBFLW23 and step BBFLW24 shown in FIG. 15B (step BBFLW65, step BBFLW66 and step BBFLW67 shown in FIG. 15D).

According to the series of processing shown in (FIG. 15A and) FIG. 15D described above, a transition of screens displayed on a monitor or the like takes place as shown in, for example, FIG. 19E, FIG. 19F or FIG. 19G.

First, the screen transition shown in FIG. 19E will be described.

When a user is in the middle of an observation or when the user releases a freeze direction, a moving image of a subject currently under observation is displayed as the endoscope image 301 (screen 1921 shown in FIG. 19E). When the user operates a key, a switch or the like having a freeze function and a freeze direction is issued, a freeze image at the timing of the direction is displayed as the endoscope image 301 (screen 1922 shown in FIG. 19E).

Then, when the record directing key is operated by the user and a release direction or a capture direction is issued from either of the states of the screen 1921 or the screen 1922 shown in FIG. 19E, the endoscope image 301 that is a freeze image is displayed as an image currently being recorded (screen 1923 shown in FIG. 19E). In other words, an image approximately matching the screen 1923 displayed on the monitor or the like is recorded onto the peripheral device.

Once recording of the image depicted on the screen 1923 shown in FIG. 19E to the peripheral device is concluded, a moving image of the subject currently under observation is once again displayed as the endoscope image 301, accompanied by a display of the thumbnail image 326 of the endoscope image 301 included in the screen 1923 shown in FIG. 19E (screen 1924 shown in FIG. 19E).

Next, the screen transition shown in FIG. 19F will be described.

When a user is in the middle of an observation or when the user releases an S freeze direction, a moving image of a subject currently under observation is displayed as the endoscope image 301 (screen 1925 shown in FIG. 19F). At the screen 1925 shown in FIG. 19F, when a key, a switch or the like having an S freeze function is operated by the user and an S freeze direction is issued, an S freeze image at the timing of the direction is displayed as the endoscope image 302, and at the same time, the moving image of the subject currently under observation is continually displayed as the endoscope image 301 (screen 1926 shown in FIG. 19F). Further, at the screen 1926 shown in FIG. 19F, when the user operates a key, a switch or the like having a freeze function and a freeze direction is issued, together with the S freeze image, a freeze image at the timing of the direction is displayed as the endoscope image 301 (screen 1928 shown in FIG. 19F).

At the screen 1925 shown in FIG. 19F, when the user operates a key, a switch or the like having a freeze function and a freeze direction is issued, a freeze image at the timing of the direction is displayed as the endoscope image 301 (screen 1927 shown in FIG. 19F). Further, at the screen 1927 shown in FIG. 19F, when the user operates a key, a switch or the like having an S freeze function and an S freeze direction is issued, together with the freeze image, an S freeze image at the timing of the direction is displayed as the endoscope image 302 (screen 1928 shown in FIG. 19F).

Then, when the record directing key is operated by the user and a release direction or a capture direction is issued from either of the states of the screen 1926 or the screen 1928 shown in FIG. 19F, the endoscope image 301 that is a freeze image and the endoscope image 302 that is an S freeze image are displayed as images currently being recorded (screen 1929 shown in FIG. 19F). In other words, images approximately matching the screen 1929 displayed on the monitor or the like are recorded onto the peripheral device.

Once recording of the images depicted on the screen 1929 shown in FIG. 19F to the peripheral device is concluded, the moving image of the subject currently under observation is once again displayed as the endoscope image 301, and as the plurality of thumbnail images 326, a thumbnail image of the endoscope image 301, a thumbnail image of the endoscope image 302, and thumbnail images originally existing in the group of thumbnail images 326A are displayed together therewith (screen 1930 shown in FIG. 19F).

Further, the screen transition shown in FIG. 19G will be described.

In addition to the screen 1928 shown in FIG. 19F, from a state of a screen 1931 that is a screen in which the endoscope form image 502 outputted from the expansion controlling unit 77B, the zoom control information 503, and the PinP image 504 outputted from the expansion controlling unit 77A are displayed together, when the record directing key is operated by the user and a release direction or a capture direction is issued, the endoscope image 301 that is a freeze image and the endoscope image 302 that is an S freeze image are displayed as images currently being recorded, and at the same time, the endoscope form image 502 and the zoom control information 503 are displayed in a state in which, for example, positions thereof are moved so as to avoid overlapping the endoscope images 301 and 302 (screen 1932 shown in FIG. 19G). In other words, images approximately matching the screen 1932 displayed on the monitor or the like are recorded onto the peripheral device.

In the present embodiment, it is assumed that the endoscope form image 502 and the zoom control information 503 that are displayed as a state of the screen 1931 are recorded together as images of the screen 1932. Display positions of the endoscope form image 502 and the zoom control information 503 may be changed based on the control of the CPU 131 by, for example, the graphic circuit 106H or by the graphic circuit 169 of the expansion controlling unit 77B. Furthermore, in the present embodiment, it is assumed that the PinP image 504 displayed as a state of the screen 1931 is not considered as an object to be recorded as an image of the screen 1932.

Once recording of the images on the screen 1932 to the peripheral device is concluded, in addition to the display content of the screen 1931, as the plurality of thumbnail images 326, a thumbnail image of the endoscope image 301, a thumbnail image of the endoscope image 302, and thumbnail images originally existing in the group of thumbnail images 326A are displayed together therewith. A record finish notification message 501 for notifying that recording of each image has been concluded (or an error occurred during recording) may also be displayed (screen 1933 shown in FIG. 19D). However, it is assumed that the endoscope image 302 that is an S freeze image which had been displayed on screen 1931 will be erased during the transition from the screen 1932 to the screen 1933.

A portion or an entirely of the endoscope form image 502, the zoom control information 503 and the PinP image 504 may be arranged to be erased at, for example, a screen after finish of recording (the screen 1933).

Now, each processing (as well as processing related to each processing) shown in FIG. 15E that is processing performed following each processing shown in FIG. 15A will be described.

The CPU 131 causes to store an endoscope composite image with a 16:9 display size in the memory 126 (step BBFLW81 shown in FIG. 15E).

Next, by the same processing as that of abovementioned step BBFLW14 shown in FIG. 15B, the CPU 131 generates a thumbnail image and sets a display position thereof (step BBFLW82 shown in FIG. 15E).

Then, by the same processing as that of abovementioned step BBFLW48 shown in FIG. 15C, the CPU 131 outputs a moving image as the endoscope image 301 by releasing still image processing (step BBFLW83 shown in FIG. 15E).

Subsequently, by approximately the same processing as that of abovementioned step BBFLW49 shown in FIG. 15C, the CPU 131 (and the CPU 151) performs processing for compressing and recording the endoscope composite image with a 16:9 display size and thumbnail images stored in the memory 126 (step BBFLW84 in FIG. 15E).

Details of the processing of step BBFLW84 in FIG. 15E will now be described with reference to the flowcharts shown in FIG. 18A and FIG. 18B. The flowcharts shown in FIG. 18A and FIG. 18B will be described under the conditions of, at the setting screen shown in FIG. 8: items ‘Release2’ and ‘Release3’ of columns ‘SDTV’ and ‘HDTV’ are set as record directing keys of the operating device, the ‘thumbnail’ item is set to ‘ON’, the ‘Peripheral device’ subitem of items ‘Release2’ and ‘Release3’ is set to the filing device 204E1, the ‘Encode’ subitem of the ‘Release2’ item is set to JPEG (or another format with a high compression rate), and the ‘Encode’ subitem of the ‘Release3’ item is set to TIFF (or another non-compressed format or a format with a low compression rate).

First, the CPU 131 detects whether the operation of the record directing key performed in step BBFLW1 shown in FIG. 15A was made from a key or a switch to which the release function of ‘Release2’ is allocated or from a key or a switch to which the release function of ‘Release3’ is allocated.

When the CPU 131 detects that the operation of the record directing key performed in step BBFLW1 shown in FIG. 15A was made from a key or a switch to which the release function of ‘Release2’ is allocated (step VFLW1 shown in FIG. 18A), the CPU 131 causes to output a freeze image to be recorded and an S freeze image to be recorded stored in the memory 126 without passing the images through the thumbnail image generating section 224, and after subjecting the images to compression/conversion in JPEG format at the compression/conversion circuit 230, stores each post-compression/conversion image to the memory 233 (step VFLW2 shown in FIG. 18A). Then, the CPU 131 once again causes to output the freeze image to be recorded and the S freeze image to be recorded stored in the memory 126, generate a thumbnail image of each image at the thumbnail image generating section 224, and after subjecting the images to compression/conversion in JPEG format at the compression/conversion circuit 230, store each post-compression/conversion image to the memory 233 (step VFLW2 shown in FIG. 18A). It is assumed that, during the processing of step VFLW2 shown in FIG. 18A, the CPU 131 performs processing by the YUV converting circuit 227 as appropriate according to contents set at the setting screen shown in FIG. 8.

The CPU 131 (or the CPU 151) then outputs the freeze image to be recorded and the S freeze image to be recorded in JPEG format which are stored in the memory 233 to the buffer 166 of the expansion controlling unit 77A (step VFLW3 shown in FIG. 18A). It is assumed that, in the processing of step VFLW3 shown in FIG. 18A, the CPU 131 (or the CPU 151) outputs each thumbnail image to the buffer 166 of the expansion controlling unit 77A together with the freeze image to be recorded and the S freeze image to be recorded in JPEG format. In the processing of step VFLW3 shown in FIG. 18A, a USB (Registered Trademark) memory, not shown, connected to the controller 164 may be used in place of the buffer 166.

Subsequently, the CPU 151 of the expansion controlling unit 77A detects whether the ‘Encryption’ item in the setting screen shown in FIG. 8 is set to ON or OFF. When the CPU 151 detects that the ‘Encryption’ item in the setting screen shown in FIG. 8 is set to ON (step VFLW4 shown in FIG. 18A), the CPU 151 subjects the freeze image to be recorded and the S freeze image to be recorded in JPEG format (as well as each thumbnail image) to encryption by the encrypting circuit 170, and outputs the encrypted freeze image to be recorded and the S freeze image to be recorded in JPEG format (as well as each thumbnail image) to the filing device 204E1 (step VFLW5 shown in FIG. 18A). When the CPU 151 detects that the ‘Encryption’ item in the setting screen shown in FIG. 8 is set to OFF (step VFLW4 shown in FIG. 18A), the CPU 151 causes to output the freeze image to be recorded and the S freeze image to be recorded in JPEG format (as well as each thumbnail image) to the filing device 204E1 (step VFLW6 shown in FIG. 18A).

When the CPU 151 detects that the output of each image to the filing device 204E1 has been concluded (step VFLW7 shown in FIG. 18A), the CPU 151 clears each image for which output has been concluded from the buffer 166 (step VFLW8 shown in FIG. 18A) and concludes processing.

In addition, when the CPU 131 detects that the operation of the record directing key performed in step BBFLW1 shown in FIG. 15A was made from a key or a switch to which the release function of ‘Release3’ is allocated (steps VFLW1 and VFLW9 shown in FIG. 18A), the CPU 131 causes to output a freeze image to be recorded and an S freeze image to be recorded stored in the memory 126 without passing the images through the thumbnail image generating section 224, and after subjecting the images to compression/conversion in TIFF format at the compression/conversion circuit 230, store each post-compression/conversion image to the memory 233 (step VFLW11 shown in FIG. 18A). Then, the CPU 131 once again causes to output the freeze image to be recorded and the S freeze image to be recorded stored in the memory 126, generate a thumbnail image of each image at the thumbnail image generating section 224, and after subjecting the images to compression/conversion in TIFF format at the compression/conversion circuit 230, store each post-compression/conversion image to the memory 233 (step VFLW10 shown in FIG. 18A). It is assumed that, during the processing of step VFLW9 shown in FIG. 18A, the CPU 131 performs processing by the YUV converting circuit 227 as appropriate according to contents set at the setting screen shown in FIG. 8.

The CPU 131 then causes to output the freeze image to be recorded and the S freeze image to be recorded in TIFF format which are stored in the memory 233 to the buffer 166 of the expansion controlling unit 77A (step VFLW11 shown in FIG. 18A), and concludes processing.

The CPU 131 may be arranged so as to perform, when causing to output each image to the buffer 166 in step VFLW3 shown in FIG. 18A and step VFLW11 shown in FIG. 18A, to process so that at least one of the respective information listed in the respective items a) to z) described above is attached to and displayed together with the image. Details of processing in which each image stored in the buffer 166 is outputted to the filing device 204E1 following the processing of VFLW11 shown in FIG. 18A will be described later.

Now, details of processing when each image stored in the buffer 166 in the abovementioned processing of step VFLW11 shown in FIG. 18A is outputted to the filing device 204E1 when, for example, a key having a function of reporting the end of examination is inputted will be described in conjunction with the flowchart shown in FIG. 18B.

When the CPU 151 of the expansion controlling unit 77A detects an input of a key having a function of reporting the end of examination, the CPU 151 reads each image stored in the buffer 166, and performs processing at the thumbnail/multi-image generating circuit 250 of the image decompressing unit 74 for generating and outputting a multi-image for displaying the respective images as a list (step VVFLW1 shown in FIG. 18B).

A specific example of the processing performed in step VVFLW1 shown in FIG. 18B is as described below.

The CPU 151 of the expansion controlling unit 77A reads each image stored in the buffer 166, so that each image in the memory 242 is stored via the bus bridge 163 and the controller 241 of the image decompressing unit 74.

The CPU 151 controls the selectors 243, 245, 246 and 248 based on, for example, information attached to each image stored in the memory 242. Consequently, according to the format or the like of each image, the CPU 151 performs decompression/conversion circuit by the decompression/conversion circuit 244 and RGB conversion by the RGB conversion circuit 247 on each image.

The CPU 151 also controls the selectors 249 and 251 so that each image outputted from the selector 248 is outputted via the thumbnail/multi-image generating circuit 250.

The thumbnail/multi-image generating circuit 250 sets the number of thumbnail images to be displayed as a list in a single screen according to the sizes of each image outputted from, for example, the selector 249, and generates and outputs a multi-image (in which, for example, 16 thumbnail images are displayed as a list in a single screen) corresponding to the number of thumbnail images.

The multi-image generated by the thumbnail/multi-image generating circuit 250 is synchronized by the synchronous circuit 252, and then outputted via the composition/masking circuit 108H or 108S (to a display unit such as a monitor), or the like.

Then, by the abovementioned processing performed in step VVFLW1 shown in FIG. 18B, a multi-image such as shown in FIG. 18D is generated and outputted.

A multi-image similar to that shown in FIG. 18D may be arranged to be generated and outputted at the thumbnail image generating section 105H and the thumbnail image generating section 105S. Also, a multi-image such as that shown in FIG. 18D may be arranged to display the group of observe information 300, the group of image related information 301A and (or) the group of image related information 302A.

The bold frame in the multi-image shown in FIG. 18D is a selection frame directing a currently selected image among the respective images included in the multi-image, and can be moved by, for example, an input of a predetermined key (such as an arrow key included in a keyboard 5 or the like) of an operating device. The selection frame is created at the graphic circuit 106H and then combined by the composition/masking processing circuit 108H, or created at the graphic circuit 106S and then combined by the composition/masking processing circuit 108S. The selection frame may also be created at the graphic circuit 169.

When the CPU 151 detects that one or more thumbnail images are selected at the multi-image shown in FIG. 18D and the selection has been finalized by an input of the select key (for example, an enter key included in the keyboard 5 or the like) (step VVFLW2 shown in FIG. 18B), the CPU 151 detects whether the ‘Encryption’ item of the setting screen shown in FIG. 8 is set to ON or OFF.

Then, when the CPU 151 detects that the ‘Encryption’ item in the setting screen shown in FIG. 8 is set to ON (step VVFLW3 shown in FIG. 18B), the CPU 151 causes to subject the freeze image to be recorded and the S freeze image to be recorded in TIFF format (as well as each thumbnail image) to encryption by the encrypting circuit 170, and output the encrypted freeze image to be recorded and the S freeze image to be recorded in TIFF format (as well as each thumbnail image) to the filing device 204E1 (step VVFLW4 shown in FIG. 18B). When the CPU 151 detects that the ‘Encryption’ item in the setting screen shown in FIG. 8 is set to OFF (step VVFLW3 shown in FIG. 18A), the CPU 151 outputs the freeze image to be recorded and the S freeze image to be recorded in JPEG format (as well as each thumbnail image) to the filing device 204E1 (step VVFLW5 shown in FIG. 18B).

When the CPU 151 detects that the output of each image to the filing device 204E1 has been concluded (step VVFLW6 shown in FIG. 18B), the CPU 151 clears each image for which output has been concluded from the buffer 166 (step VVFLW7 shown in FIG. 18B), and concludes processing.

The CPU 151 may be arranged, for example, to causes to output all images recorded onto the buffer 166 (to the filing device 204E1) instead of performing the processing of step VVFLW1 and step VVFLW2 shown in FIG. 18B.

Now, details of processing in the case where each image stored in the buffer 166 in the abovementioned processing of step VFLW11 shown in FIG. 18A is outputted to the filing device 204B1 when, for example, the power of the processor 4 is turned on will be described in conjunction with the flowchart shown in FIG. 18C.

The CPU 151 detects whether or not uncleared images are stored in the buffer 166 when the power of the processor is turned on. When the CPU 151 detects that uncleared images are not stored in the buffer 166 when the power of the processor is turned on (step VVVFLW1 shown in FIG. 18C), the CPU 151 concludes processing.

When the CPU 151 detects that uncleared images are stored in the buffer 166 when the power of the processor is turned on (step VVVFLW1 shown in FIG. 18C), the CPU 151 the further detects whether the ‘Encryption’ item in the setting screen shown in FIG. 8 is set to ON or OFF.

When the CPU 151 detects that the ‘Encryption’ item in the setting screen shown in FIG. 8 is set to ON (step VVVFLW2 shown in FIG. 18C), the CPU 151 subjects the freeze image to be recorded and the S freeze image to be recorded in TIFF format (as well as each thumbnail image) to encryption by the encrypting circuit 170, and outputs the encrypted freeze image to be recorded and the S freeze image to be recorded in TIFF format (as well as each thumbnail image) to the filing device 204E1 (step VVVFLW3 shown in FIG. 18C). When the CPU 151 detects that the ‘Encryption’ item in the setting screen shown in FIG. 8 is set to OFF (step VVVFLW2 shown in FIG. 18C), the CPU 151 causes to output the freeze image to be recorded and the S freeze image to be recorded in JPEG format (as well as each thumbnail image) to the filing device 204E1 (step VVVFLW4 shown in FIG. 18C).

Subsequently, the CPU 151 clears each image for which output has been concluded from the buffer 166 (step VVVFLW5 shown in FIG. 18C) and concludes processing.

The CPU 151 may be arranged to perform after the processing of step VVVFLW1 shown in FIG. 18C processing for causing to generate a multi-image for showing a list of images not cleared from the buffer 166 by, for example, to process similar to that performed in step VVFLW1 shown in FIG. 18B.

According to the series of processing shown in (FIG. 15A and) FIG. 15E described above, a transition of screens displayed on a monitor or the like takes place as shown in, for example, FIG. 19H.

The screen transition shown in FIG. 19H will now be described.

In addition to the screen 1928 shown in FIG. 19F, from a state of a screen 1934 that is a screen in which the endoscope form image 502 outputted from the expansion controlling unit 77B, the zoom control information 503, and the Pinp image 504 outputted from the expansion controlling unit 77A are displayed together, when the record directing key is operated by the user and a release direction or a capture direction is issued, images of the screen 1935 including the endoscope image 301 and the endoscope image 302, the endoscope form image 502 in a state in which the position thereof has been moved, and the zoom control information 503 is recorded onto the peripheral device. Further, prior to the recording of the images on the screen 1935 shown in FIG. 19G, processing of abovementioned step BBFLW83 shown in FIG. 15E is performed to release freezing of an image being outputted to the monitor. Consequently, a moving image of the subject currently under observation is displayed as the endoscope image 301, and the endoscope form image 502, the zoom control information 503 and the PinP image 504 are displayed in a state in which, for example, positions thereof are moved so as to avoid overlapping the endoscope image 301 (screen 1936 shown in FIG. 19G). In other words, images of the screen 1935 that is different from the screen 1936 displayed on the monitor or the like is recorded onto the peripheral device. In the present embodiment, it is assumed that the PinP image 504 displayed as a state of the screen 1934 is not considered to be an object to be recorded as an image of the screen 1935. On the screen 1936 shown in FIG. 19H, as the plurality of thumbnail images 326, a thumbnail image of the endoscope image 301, a thumbnail image of the endoscope image 302, and thumbnail images originally existing in the group of thumbnail images 326A are displayed together with the respective abovementioned images.

Once recording of the images of the screen 1935 to the peripheral device is concluded, in addition to the display content of the screen 1936, a record finish notification message 501 for notifying that recording of each image has been concluded (or an error occurred during recording) is displayed (screen 1937 shown in FIG. 19H).

A portion or an entirely of the endoscope form image 502, the zoom control information 503 and the PinP image 504 may be arranged to be erased at, for example, a screen after finish of recording (the screen 1937).

At ‘Peripheral device’ that is a subitem of any one of items ‘Release1’ to ‘Release4’ in the setting screen shown in FIG. 8, when a plurality of devices including a device corresponding to recorded image display mode and a device not corresponding to recorded image display mode has been set, the CPU 131 may be arranged to perform compression and recording similar to each processing shown in FIG. 18A after performing, for example, the processing of step BBFLW24 shown in FIG. 15B or the processing of step BBFLW67 shown in FIG. 15D.

Now, processing performed by each section of the processor 4 when a freeze direction or an S freeze direction has been issued will be described.

First, the CPU 131 of the main controlling unit 75 detects whether a freeze direction or an S freeze direction has been issued at any one of the respective operating devices. When the CPU 131 detects that a freeze direction has been issued at any one of the respective operating devices (step SFLW1 shown in FIG. 20A), the CPU 131 successively performs processing of step SFLW3 and thereafter shown in FIG. 20A to be described later. When the CPU 131 detects that an S freeze direction has been issued at any one of the respective operating devices (step SFLW1 and step SFLW2 shown in FIG. 20A), the CPU 131 successively performs processing of step SFLW21 and thereafter shown in FIG. 20B to be described later.

After detecting that a freeze direction has been issued at any one of the respective operating devices, the CPU 131 detects what kind of image is currently being outputted to the display unit such as a monitor or the like. For example, as is the case with the screen 1901 shown in FIG. 19A, the screen 1921 shown in FIG. 19E or the screen 1925 shown in FIG. 19F, when the CPU 131 detects that only a moving image is being outputted to the display unit such as a monitor (step SFLW3 shown in FIG. 20A), the CPU 131 successively performs the processing of step SFLW6 shown in FIG. 20A to be described later. As is the case with the screen 1902 shown in FIG. 19A, the screen 1922 shown in FIG. 19E or the screen 1927 shown in FIG. 19F, when the CPU 131 detects that a freeze image is being outputted to the display unit such as a monitor (step SFLW3 and step SFLW4 shown in FIG. 20A), the CPU 131 successively performs the processing of step SFLW9 shown in FIG. 20A to be described later. As is the case with, for example, the screen 1906 shown in FIG. 19B or the screen 1926 shown in FIG. 19F, when the CPU 131 detects that a moving image and an S freeze image are being outputted to the display unit such as a monitor (step SFLW3, step SFLW4 and step SFLW5 shown in FIG. 20A), the CPU 131 successively performs the processing of step SFLW11 shown in FIG. 20A to be described later. Furthermore, as is the case with, for example, the screen 1907 shown in FIG. 19B or the screen 1928 shown in FIG. 19F, when the CPU 131 detects that a freeze image and an S freeze image are being outputted to the display unit such as a monitor (step SFLW3, step SFLW4 and step SFLW5 shown in FIG. 20A), the CPU 131 successively performs the processing of step SFLW14 shown in FIG. 20A to be described later.

When a freeze direction is issued in the case where only a moving image is being outputted to the display unit such as a monitor, the CPU 131 causes to generate a freeze image at the freeze circuit 96 and to perform pre-freeze processing (step SFLW6 shown in FIG. 20A). By controlling the post-stage image processing circuit 98 in addition to the abovementioned processing of step SFLW6 shown in FIG. 20A, the CPU 131 calculates an IHb average of the freeze image.

Subsequently, by controlling the graphic circuit 106H, for example, the CPU 131 causes to update respective information (the changes the value of the hemoglobin index 322A and the like) included in the group of observe information 300 and the group of image related information 301A according to the IHb average or the like calculated at the post-stage image processing circuit 98 (step SFLW7 shown in FIG. 20A).

Then, by controlling the composition/masking processing circuit 108H, the CPU 131 causes to combine and output the freeze image generated at the freeze circuit 96, the group of observe information 300 and the group of image related information 301A updated at the graphic circuit 106H, and each thumbnail image 326 generated at the thumbnail image generating circuit 105H (step SFLW8 shown in FIG. 20A). Consequently, a screen such as, for example, the screen 1902 shown in FIG. 19A, the screen 1922 shown in FIG. 19E or the screen 1927 shown in FIG. 19F is displayed on the display unit such as a monitor. (Images that have been displayed prior to the issuing of the freeze direction among the endoscope form image 502, the zoom control information 503 and the PinP image 504 are additionally combined and outputted in the processing of step SFLW8 shown in FIG. 20A).

When a freeze direction is issued in the case where a freeze image is being outputted to the display unit such as a monitor, the CPU 131 causes to release the freeze state by interrupting the generation of the freeze image at the freeze circuit 96 (step SFLW9 shown in FIG. 20A). Consequently, the CPU 131 outputs a moving image to the display unit such as a monitor. By controlling the graphic circuit 106H in addition to the abovementioned processing of step SFLW9 shown in FIG. 20A, the CPU 131 updates respective information included in the group of observe information 300 and the group of image related information 301A (for example, changes the display of the hemoglobin index 322A to ‘---’ or the like).

Then, by controlling the composition/masking processing circuit 108H, the CPU 131 combines and outputs the moving image, the group of observe information 300 and the group of image related information 301A updated at the graphic circuit 106H, and each thumbnail image 326 generated at the thumbnail image generating circuit 105H (step SFLW10 shown in FIG. 20A). Consequently, a screen such as, for example, the screen 1901 shown in FIG. 19A, the screen 1921 shown in FIG. 19E or the screen 1925 shown in FIG. 19F is displayed on the display unit such as a monitor. (Images that have been displayed prior to the issuing of the freeze direction among the endoscope form image 502, the zoom control information 503 and the PinP image 504 are additionally combined and outputted in the processing of step SFLW10 shown in FIG. 20A).

When a freeze direction is issued in the case where a moving image and an S freeze image are being outputted to the display unit such as a monitor, the CPU 131 causes to generate a freeze image at the freeze circuit 96 and performs pre-freeze processing (step SFLW11 shown in FIG. 20A). By controlling the post-stage image processing circuit 98 in addition to the abovementioned processing of step SFLW11 shown in FIG. 20A, the CPU 131 calculates an IHb average of the freeze image.

Subsequently, by controlling the graphic circuit 106H, for example, the CPU 131 causes to update respective information included in the group of observe information 300 and the group of image related information 301A according to the IHb average or the like calculated at the post-stage image processing circuit 98 (for example, changes the value of the hemoglobin index 322A or the like) (step SFLW12 shown in FIG. 20A).

Then, by reading the S freeze image from the memory 112H and by controlling the composition/masking processing circuit 108H, the CPU 131 causes to combine and output the S freeze image, the group of image related information 302A related to the S freeze image, the freeze image generated at the freeze circuit 96, the group of observe information 300 and the group of image related information 301A updated at the graphic circuit 106H, and each thumbnail image 326 generated at the thumbnail image generating circuit 105H (step SFLW13 shown in FIG. 20A). Consequently, a screen such as, for example, the screen 1907 shown in FIG. 19B or the screen 1928 shown in FIG. 19F is displayed on the display unit such as a monitor. (Images that have been displayed prior to the issuing of the freeze direction among the endoscope form image 502, the zoom control information 503 and the PinP image 504 are additionally combined and outputted in the processing of step SFLW13 shown in FIG. 20A).

When a freeze direction is issued in the case where a freeze image is being outputted to the display unit such as a monitor, the CPU 131 releases the freeze state by interrupting the generation of the freeze image at the freeze circuit 96 (step SFLW14 shown in FIG. 20A). Consequently, the CPU 131 outputs a moving image to the display unit such as a monitor. By controlling the graphic circuit 106H in addition to the abovementioned processing of step SFLW14 shown in FIG. 20A, the CPU 131 updates respective information included in the group of observe information 300 and the group of image related information 301A (for example, changes the display of the hemoglobin index 322A to ‘---’ or the like).

Then, by reading the S freeze image from the memory 112H and by controlling the composition/masking processing circuit 108H, the CPU 131 causes to combine and output the S freeze image, the group of image related information 302A related to the S freeze image, the moving image, the group of observe information 300 and the group of image related information 301A updated at the graphic circuit 106H, and each thumbnail image 326 generated at the thumbnail image generating circuit 105H (step SFLW15 shown in FIG. 20A). Consequently, a screen such as, for example, the screen 1906 shown in FIG. 19B or the screen 1926 shown in FIG. 19F is displayed on the display unit such as a monitor. (Images that have been displayed prior to the issuing of the freeze direction among the endoscope form image 502, the zoom control information 503 and the PinP image 504 are additionally combined and outputted in the processing of step SFLW15 shown in FIG. 20A).

After detecting that an S freeze direction has been issued at any one of the respective operating devices, the CPU 131 detects what kind of image is currently being outputted to the display unit such as a monitor or the like. For example, as is the case with the screen 1905 shown in FIG. 19B or the screen 1925 shown in FIG. 19F, when the CPU 131 detects that only a moving image is being outputted to the display unit such as a monitor (step SFLW21 shown in FIG. 20B), the CPU 131 successively performs the processing of step SFLW24 shown in FIG. 20B to be described later. For example, as is the case with the screen 1927 shown in FIG. 19F, when the CPU 131 detects that a freeze image is being outputted to the display unit such as a monitor (step SFLW21 and step SFLW22 shown in FIG. 20B), the CPU 131 successively performs the processing of step SFLW29 shown in FIG. 20B to be described later. For example, as is the case with the screen 1906 shown in FIG. 19B or the screen 1926 shown in FIG. 19F, when the CPU 131 detects that a moving image and an S freeze image are being outputted to the display unit such as a monitor (step SFLW21, step SFLW22 and step SFLW23 shown in FIG. 20B), the CPU 131 successively performs the processing of step SFLW32 shown in FIG. 20B to be described later. Furthermore, for example, as is the case with the screen 1907 shown in FIG. 19B or the screen 1928 shown in FIG. 19F, when the CPU 131 detects that a freeze image and an S freeze image are being outputted to the display unit such as a monitor (step SFLW21, step SFLW22 and step SFLW23 shown in FIG. 20B), the CPU 131 successively performs the processing of step SFLW shown in FIG. 20B to be described later.

When an S freeze direction is issued in the case where only a moving image is being outputted to the display unit such as a monitor, the CPU 131 causes to generate a freeze image at the freeze circuit 96 and perform pre-freeze processing (step SFLW24 shown in FIG. 20B). By controlling the post-stage image processing circuit 98 in addition to the abovementioned processing of step SFLW24 shown in FIG. 20B, the CPU 131 calculates an IHb average of the freeze image.

The CPU 131 causes to output the freeze image generated at the freeze circuit 96 to the HDTV-side processing system (the zoom-up/highlight circuit 99H and thereafter), and controls the composition/masking processing circuit 108H so that the freeze image is stored as an S freeze image to the memory 112H (step SFLW25 shown in FIG. 20B).

Then, the CPU 131 releases the freeze state by interrupting the generation of the freeze image at the freeze circuit 96 (step SFLW26 shown in FIG. 20B). Consequently, the CPU 131 causes to output a moving image to the display unit such as a monitor.

Subsequently, by controlling the graphic circuit 106H, for example, the CPU 131 causes to output respective information included in the group of observe information 300 and the group of image related information 302A (such as changing the value of the hemoglobin index 322B or the like) according to the IHb average or the like calculated at the post-stage image processing circuit 98 (step SFLW27 shown in FIG. 20B).

Then, by reading the S freeze image from the memory 112H and by controlling the composition/masking processing circuit 108H, the CPU 131 causes to combine and output the S freeze image, the group of observe information 300 and the group of image related information 302A updated at the graphic circuit 106H, the moving image, the group of image related information 301A related to the moving image, and each thumbnail image 326 generated at the thumbnail image generating circuit 105H (step SFLW28 shown in FIG. 20B). Consequently, a screen such as, for example, the screen 1906 shown in FIG. 19B or the screen 1926 shown in FIG. 19F is displayed on the display unit such as a monitor. (Images that have been displayed prior to the issuing of the S freeze direction among the endoscope form image 502, the zoom control information 503 and the PinP image 504 are additionally combined and outputted in the processing of step SFLW28 shown in FIG. 20B).

When an S freeze direction is issued in the case where a freeze image is being outputted to the display unit such as a monitor, the CPU 131 causes to output the freeze image newly generated at the freeze circuit 96 to the HDTV-side processing system (the zoom-up/highlight circuit 99H and thereafter), and control the composition/masking processing circuit 108H to store the newly generated freeze image as an S freeze image to the memory 112H (step SFLW29 shown in FIG. 20B). By controlling the post-stage image processing circuit 98 in addition to the abovementioned processing of step SFLW29 shown in FIG. 20B, the CPU 131 causes to calculate an IHb average of the newly generated freeze image.

Subsequently, by controlling the graphic circuit 106H, for example, the CPU 131 causes to update respective information included in the group of observe information 300 and the group of image related information 302A (such as changing the value of the hemoglobin index 322B or the like) according to the IHb average or the like calculated at the post-stage image processing circuit 98 (step SFLW30 shown in FIG. 20B).

Then, by reading the S freeze image from the memory 112H and by controlling the composition/masking processing circuit 108H, the CPU 131 causes to combine and output the S freeze image, the group of observe information 300 and the group of image related information 302A updated at the graphic circuit 106H, the freeze image, the group of image related information 301A related to the freeze image, and each thumbnail image 326 generated at the thumbnail image generating circuit 105H (step SFLW31 shown in FIG. 20B). Consequently, a screen such as, for example, the screen 1907 shown in FIG. 19B or the screen 1928 shown in FIG. 19F is displayed on the display unit such as a monitor. (Images that have been displayed prior to the issuing of the S freeze direction among the endoscope form image 502, the zoom control information 503 and the PinP image 504 are additionally combined and outputted in the processing of step SFLW31 shown in FIG. 20B).

When an S freeze direction is issued in the case where a moving image and an S freeze image are being outputted to the display unit such as a monitor, the CPU 131 causes to generate a freeze image at the freeze circuit 96 and perform pre-freeze processing (step SFLW32 shown in FIG. 20B). By controlling the post-stage image processing circuit 98 in addition to the abovementioned processing of step SFLW32 shown in FIG. 20B, the CPU 131 causes to calculate an IHb average of the freeze image.

The CPU 131 causes to output the freeze image newly generated at the freeze circuit 96 to the HDTV-side processing system (the zoom-up/highlight circuit 99H and thereafter), and control the composition/masking processing circuit 108H to store the newly generated freeze image as the newest S freeze image to the memory 112H (step SFLW33 shown in FIG. 20B).

Then, the CPU 131 causes to release the freeze state by interrupting the generation of the freeze image at the freeze circuit 96 (step SFLW34 shown in FIG. 20B). Consequently, the CPU 131 causes to output a moving image to the display unit such as a monitor.

Subsequently, by controlling the graphic circuit 106H, for example, the CPU 131 causes to update respective information included in the group of observe information 300 and the group of image related information 302A (such as changing the value of the hemoglobin index 322B or the like) according to the IHb average or the like calculated at the post-stage image processing circuit 98 (step SFLW35 shown in FIG. 20B).

Then, by reading the newest S freeze image from the memory 112H and by controlling the composition/masking processing circuit 108H, the CPU 131 causes to combine and output the newest S freeze image, the group of observe information 300 and the group of image related information 302A updated at the graphic circuit 106H, the moving image, the group of image related information 301A related to the moving image, and each thumbnail image 326 generated at the thumbnail image generating circuit 105H (step SFLW36 shown in FIG. 20B). Consequently, a screen such as, for example, the screen 1906 shown in FIG. 19B or the screen 1926 shown in FIG. 19F is displayed on the display unit such as a monitor. (Images that have been displayed prior to the issuing of the S freeze direction among the endoscope form image 502, the zoom control information 503 and the PinP image 504 are additionally combined and outputted in the processing of step SFLW36 shown in FIG. 20B).

When an S freeze direction is issued in the case where a freeze image and an S freeze image are being outputted to the display unit such as a monitor, the CPU 131 causes to output the freeze image newly generated at the freeze circuit 96 to the HDTV-side processing system (the zoom-up/highlight circuit 99H and thereafter), and controls the composition/masking processing circuit 108H to store the newly generated freeze image as the newest S freeze image to the memory 112H (step SFLW37 shown in FIG. 20B).

Subsequently, by controlling the graphic circuit 106H, for example, the CPU 131 causes to update respective information included in the group of observe information 300 and the group of image related information 302A (such as changing the value of the hemoglobin index 322B or the like) according to the IHb average or the like calculated at the post-stage image processing circuit 98 (step SFLW38 shown in FIG. 20B).

Then, by reading the newest S freeze image from the memory 112H and by controlling the composition/masking processing circuit 108H, the CPU 131 causes to combine and output the newest S freeze image, the group of observe information 300 and the group of image related information 302A updated at the graphic circuit 106H, the freeze image, the group of image related information 301A related to the freeze image, and each thumbnail image 326 generated at the thumbnail image generating circuit 105H (step SFLW39 shown in FIG. 20B). Consequently, a screen such as, for example, the screen 1907 shown in FIG. 19B or the screen 1928 shown in FIG. 19F is displayed on the display unit such as a monitor. (Images that have been displayed prior to the issuing of the S freeze direction among the endoscope form image 502, the zoom control information 503 and the PinP image 504 are additionally combined and outputted in the processing of step SFLW39 shown in FIG. 20B).

A key or a switch provided with an S freeze function is not limited to that allocated at the setting screen shown on FIG. 8, and may be configured so that, for example, the CPU 131 detects that an S freeze direction has been issued when a key or a switch provided with a freeze function is pressed for a predetermined period or longer, or may be configured so that, for example, the CPU 131 detects that the S freeze direction has been issued when a key or a switch provided with a freeze function is consecutively pressed within a predetermined period or longer.

In the present embodiment, a standard to be used for outputting images (still images and moving images) may be, for example, a standard conforming to any of the digital transmission standards ITU-R BT.656, BT.601, BT.709, BT.799, BT.1120, BT.1364 and BTA S-001, BTA S-002, BTA S-004, BTA S-005.

As described above, the processor 4 of the endoscope system 1 is capable of outputting an image suitable for recording even when an image with a 16:9 display size is displayed on a monitor or the like and the image is to be recorded on a device that does not accommodate the display size. Consequently, the processor 4 of the endoscope system 1 can reduce the burden placed on the user when recording an endoscope image.

As described above, the processor 4 of the endoscope system 1 is configured so as to be capable of setting at the setting screen shown in FIG. 8, when a key (or switch) provided with a release function is inputted, a peripheral device onto which an image is to be recorded and a format or the like to be used when the image is subjected to compression for each key (or switch) that is an object of allocation of the release function. Consequently, for example, by separately using a key or a switch for recording an image with a high compression rate and a key or a switch for recording an uncompressed image or an image with a low compression rate as keys or switches provided with a release function as shown in FIG. 18A, even when the user is currently performing an observation, the processor 4 of the endoscope system 1 is capable of selecting an image format and a compression rate in a simple manner within a short period of time without interrupting the observation. The processor 4 of the endoscope system 1 is also capable of consecutively recording images (onto a peripheral device or the like) in realtime when a format at a high compression rate is selected.

Furthermore, as described above, the processor 4 of the endoscope system 1 is provided with, for example, a function for storing an image in a format of low compression rate in the buffer 166 as shown in FIG. 18B, and at the same time, outputting only an image selected by the user at a predetermined timing. Consequently, the processor 4 of the endoscope system 1 is capable of reducing the transmission load that occurs when an image in a format of low compression rate is transmitted over a network.

As described above, the processor 4 of the endoscope system 1 is capable of automatically detecting connections of the expansion controlling units 77A and 77B configured as expansion boards, and based on the detection result, displaying an image or information related to the functions of the connected expansion boards immediately after the connection of the expansion controlling units 77A and 77B. As a result, the processor 4 of the endoscope system 1 is capable of reducing the time required by the user for observation compared to before.

As described above, since the processor 4 of the endoscope system 1 is capable of performing encryption on an image to be recorded, for example, it is possible to prevent the image from being displayed at a device not equipped with a decryption mechanism. As a result, the user is capable of securely implementing security measures on patient information and protection of personal information.

It should be obvious that the present invention is not limited to the embodiments described above, and various modifications and applications may be made without departing from the scope thereof.

What is claimed is:

1. Medical image processing apparatus comprising:
an image compressing unit for compressing a medical image according to a subject's image taken by an image pickup unit with either a first image compressing method or a second image compressing method different from the first image compressing method;
a record direction input setting unit for allocating a first record directing section that is capable of performing a first record direction for compressing the medical image with the first image compressing method and recording the image and a second record directing section that is capable of performing a second record direction for compressing the medical image with the second image compressing method and recording the image to any of a plurality of keys or switches; and
a controlling unit for outputting the medical image that is compressed with the first image compressing method to an image recording unit in response to detection of performance of the first record direction and outputting the medical image that is compressed with the second image compressing method to the image recording unit in response to detection of performance of the second record direction.

2. The medical image processing apparatus according to claim 1, wherein the second image compressing method employs a compression rate lower than that of the first image compressing method or non-compression.

3. The medical image processing apparatus according to claim 1, further comprising:
a buffer for temporarily recording the medial image compressed with the second image compressing method; wherein
the controlling unit outputs the image recorded in the buffer to the image recording unit when the controlling unit detects that a predetermined report has given to the medial image processing apparatus.

4. The medical image processing apparatus according to claim 3, further comprising:
a multi image generating unit for generating a multi image, which is capable of list a plurality of images inputted; wherein
the controlling unit causes the multi image generating unit to generate a multi image based on each image recorded in the buffer and outputs an image selected in the multi image among images recorded in the buffer to the image recording unit when the controlling unit detects that a predetermined report is issued to the medical image processing apparatus.

5. The medical image processing apparatus according to claim 3, wherein
after the medical image processing apparatus is switched on again, the controlling unit outputs the image recorded in the buffer to the image recording unit.

6. The medical image processing apparatus according to claim 3, wherein
the controlling unit outputs the image recorded in the buffer to the image recording unit and then, deletes the image recorded in the buffer.

7. An endoscope system comprising:
a endoscope for picking up an image of a subject;
medical image processing apparatus for obtaining an endoscope image according to the subject's image;
at least one expansion controlling unit that can be connected to the medical image processing apparatus and that enables at least a predetermined function related with either the endoscope or the medical image processing apparatus when the expansion controlling unit is connected to the medical image processing apparatus;
a connection information storing unit that is provided for each expansion controlling unit for storing different types of connection detection information according to the type of the expansion controlling unit; and
a main controlling unit for determining the type of the expansion controlling unit that is connected to the medical image processing apparatus based on the connection detection information stored in each connection information storing unit and outputting an image or information related with the predetermined function according to the determined result to a display unit.

8. The endoscope system according to claim 7, wherein
the main controlling unit controls to display the image or the information related with the predetermined function at a predetermined position on a display screen of the display unit.

9. The endoscope system according to claim 7, wherein
the main controlling unit outputs the image or the information related with the predetermined function to the display unit only when the main controlling unit can detect the connection detection information stored in each connection information storing unit.

10. The endoscope system according to claim 7, wherein
each of the at least one expansion controlling unit has a configuration for enabling at least any one of a network communication function that enables a peripheral device to be connected with the medical image processing apparatus via a network, an endoscope inserting form detecting function that enables the endoscope inserting form to be detected, and a zoom-controlling function that enables an image pickup state for the endoscope to pickup an image of the subject to a desired zoom state, as the predetermined function.

11. The endoscope system according to claim 8, wherein
the predetermined position is any of the upper left, the lower left, the upper right and the lower right on the display screen.

12. A medical image processing system comprising:
an endoscope for picking up an image of a subject;
medical image processing apparatus that is capable of outputting a first medical image according to the subject's image on an image region in a display unit and outputting a second medical image according to the subject's image on an image region different from the first image region in the display unit;
an image recording unit that is capable of recording the first medical image and the second medical image; and
a controlling unit for selecting between recording the first medical image and the second medical image as an image on a screen, or recording them as images on different screens according to an outputted size of an image that can be recorded by the image recording unit.

13. The medical image processing system according to claim 12, wherein
the controlling unit generates a first medical image to be recorded including the first medical image and a second medical image to be recorded including the second medical image and causes the image recording unit to record the first medical image to be recorded and the second medical image to be recorded when the controlling unit causes the first medical image and the second medical image to be recorded as images on different screens.

14. The medical image processing system according to claim 13, wherein
the first medical image to be outputted and the second medical image to be outputted which are generated by the controlling unit have at least either time information that is information on a time when a record direction for the first medical image and the second medical image is issued or subject information that is information on a subject from which the first medical image and the second medical image are obtained.

15. The medical image processing system according to claim 13, wherein
the controlling unit causes the first medical image to be recorded to be outputted to the display unit while the first medical image to be outputted is being recorded in the image recording unit, and causes the second medical image to be recorded to be outputted to the display unit while the second medical image to be outputted is being recorded in the image recording unit.

16. The medical image processing system according to claim 12, wherein
the controlling unit generates and outputs a first thumbnail image according to the first medical image and a second thumbnail image according to the second medical image to record the first medical image and the second medical image.

17. The medical image processing system according to claim 1, wherein
a peripheral device to be connected to the medical image processing apparatus comprises:
a common information storing unit that stores latest setting information of the peripheral device, latest setting information of a peripheral device other than the peripheral device, and date and time information of the date and time when each of the setting information is updated;
a log information storing unit that stores an update history of the setting information of the peripheral device; and
a setting information update controlling unit for updating information stored in the common information storing unit and the log information storing unit each time when setting of the peripheral device is updated;
wherein when the setting information update controlling unit detects that the peripheral devices is rebooted or that the peripheral device is switched on again and if a difference between the date and time of when the setting information of the peripheral device is written and the current date and time shown at a time report unit is within a predetermined period based on the date and time information stored in the common information storing unit, the setting information update controlling unit takes setting based on the setting information stored in the common information storing unit as setting of the peripheral device, and if the difference between the date and time of when the setting information of the peripheral device is written and current date and time shown at the time report unit is over the predetermined period, the setting information update controlling unit initializes the setting of the peripheral device and also updates the setting information stored in the common information storing unit to a value at the initialization.
